(19) United States
(12) Patent Application Publication
(10) Pub. No.: US 2022/0030866 A1
(43) Pub. Date: Feb. 3, 2022

(54) CONJUGATES OF AUXIN ANALOGS

(71) Applicants: The State of Israel, Ministry of Agriculture & Rural Development, Agricultural Research Organization, Rishon-Lezion (IL); Ramot at Tel-Aviv University Ltd., Tel-Aviv (IL); Yissum Research Development Company of the Hebrew University of Jerusalem Ltd., Jerusalem (IL)

(72) Inventors: Einat SADOT, Moshav Sitriya (IL); Roy WEINSTAIN, Tel-Aviv (IL); Joseph RIOV, Petach-Tikva (IL); Leor ESHED-WILLIAMS, Moshav Kidron (IL)

(73) Assignees: The State of Israel, Ministry of Agriculture & Rural Development, Agricultural Research Organization, Rishon-Lezion (IL); Ramot at Tel-Aviv University Ltd., Tel-Aviv (IL); Yissum Research Development Company of the Hebrew University of Jerusalem Ltd., Jerusalem (IL)

(21) Appl. No.: 17/503,581

(22) Filed: Oct. 18, 2021

Related U.S. Application Data
(63) Continuation of application No. PCT/IL2020/050453, filed on Apr. 16, 2020.
(60) Provisional application No. 62/885,840, filed on Aug. 13, 2019.

(30) Foreign Application Priority Data
Apr. 18, 2019 (IL) 266136

(51) Int. Cl. A01N 43/40; A01G 7/06; A01N 43/38; A01N 37/18

(52) U.S. Cl. CPC A01N 43/40; A01N 37/18; A01N 43/38; A01G 7/06

(57) ABSTRACT

Described herein are methods of enhancing formation and/or growth of an adventitious root in a plant and/or plant tissue, and/or for promoting grafting unification, enhancing fruit size and/or reducing flowering in a plant. The method comprises contacting at least a portion of the plant and/or plant tissue with a compound having Formula I:

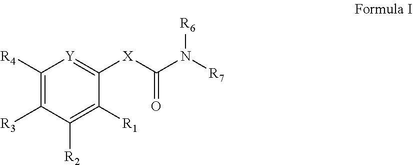

wherein X, Y and R₁-R₇ are as defined herein. Further described are compositions for enhancing formation and/or growth of an adventitious root in a plant and/or plant tissue, and/or for promoting grafting unification, enhancing fruit size and/or reducing flowering in a plant, comprising the abovementioned compound and a horticulturally acceptable carrier. Novel compounds having Formula I are also described herein.

### RELATED APPLICATIONS

This application is a Continuation of PCT Patent Application No. PCT/IL2020/050453, having international filing date of Apr. 16, 2020 which claims the benefit of priority of Israel Patent Application No. 266136 filed on Apr. 18, 2019, and under 35 USC § 119(e) of U.S. Provisional Patent Application No. 62/885,840 filed on Aug. 13, 2019. The contents of the above applications are all incorporated by reference as if fully set forth herein in their entirety.

### FIELD AND BACKGROUND OF THE INVENTION

The present invention, in some embodiments thereof, relates to treatment of plants, and more particularly, but not exclusively, to compounds useful for inducing root formation in plants, such as in plant cuttings, and for promoting grafting unification, enhancing fruit size and reducing flowering.

Adventitious roots (ARs) are roots that regenerate from non-root tissues, in contrast to lateral roots that are post-embryonic roots formed from roots [Verstraeten et al., Front Plant Sci 2014, 5:495]. ARs can develop from natural preformed primordia, such as in rice [Steffens et al., Plant Cell 2012, 24:3296-3306] or sweet potato [Firon et al., in: The Sweet Potato, Loebenstein & Thottappilly Eds., Springer, Dordrecht, pp. 13-16 (2009)], or after naturally occurring damage such as in waterlogging [Sauter, Curr Opin Plant Biol 2013, 16:282-286], or due to wounding during cutting preparation. In all cases the plant hormone auxin is involved in AR induction.

However, difficulties arise with the need to propagate clones of recalcitrant plants which have lost their ability to form ARs during maturation or which are genetically difficult to root. Clonal propagation of plants by induction of ARs in stem cuttings is an important step in breeding programs and in agricultural practice; and increasing rooting efficiency in terms of percentage, time, and uniformity is a major goal in agriculture and has considerable economic consequences.

The mechanism which prevents AR formation in recalcitrant plants has therefore been the target of many studies, yet much remains unclear.

Loss of rooting capability is common in woody plants such as forest trees, rootstocks for fruit trees, and ornamental plants. Gradual loss of rooting capability often occurs in woody plants in association with maturation and flowering acquisition (which indicates completion of the maturation process) [Hackett, Hort Rev 1985, 7:109-155; Poethig, Science 1990, 250:923-930; Poethig, Plant Physiol 2010, 154:541-544].

It has been reported that loss of rooting capability precedes the maturation stage in Eucalyptus trees with grayish leaves, such as Eucalyptus brachyphylla or E. cinerea [Levy et al., BMC Genomics 2014, 15:524]. This suggests that although maturation may contribute to loss of rooting capability, maturation is not the only biological process influencing rooting capability [Riov et al., in: Plant Roots: The Hidden Half, 4th ed., Eshel, A. and Beeckman T., eds. Taylor & Francis pp. 11.11-11.14 (2013)].

Auxins are a class of plant hormones, either natural or synthetic, which are involved in various processes of plant growth and development. Auxins have been commonly used to promote rooting of cuttings or shootlets (in combination with cytokinins) in tissue culture. Of the large number of auxins, indole-3-acetic acid (IAA), indole-3-butyric acid (IBA), and 1-naphthaleneacetic acid (NAA), sometimes in combination, are the most used auxins for this purpose [Hartmann et al., Hartmann and Kester's Plant Propagation Principles and Practices, Eighth Edition, Pearson Education Limited, Essex, Great Britain (2011)]. IAA and IBA are natural auxins and NAA is a synthetic auxin.

IBA and NAA, as well as the amide of NAA (1-naphthaleneacetamide), are used to promote root initiation and growth.

Exogenous IAA and IBA has been reported to be rapidly metabolized in plant tissues, with conjugation to amino acids or glucose being the major pathway of IAA metabolism [Cohen & Bandurski, Annu Rev Plant Physiol 1982, 33:403-430; Hangarter & Good, Plant Physiol 1981, 68:1424-1427; Wiesman et al., Physiologia Plantarum 1988, 74:556-560; Wiesman et al., Plant Physiol 1989, 91:1080-1084]. It has been hypothesized that auxin conjugates are a storage form of auxin, from which free active auxin can be released [Riov, Acta Hort 1993, 329:284-288; Ludwig-Muller, J Exp Bot 2011, 62:1757-1773].

The possible use of auxin conjugates to promote rooting has been examined in several studies. Haissig [Physiol Plant 1979, 47:29-33] reported that phenyl esters of IAA and IBA were more active than the free auxins in inducing adventitious root formation and development. Other studies reported rooting potential of IAA and IBA conjugates, mostly with amino acids. The alanine conjugate of IBA was reported to efficiently promoted rooting in highbush blueberries (Vaccinium corymbosum L.) cuttings [Mihaljevic & Salopek-Sondi, Plant Soil Environ 2012, 58:236-241]; whereas IBA-phenylalanine, IBA-alanine, IAA-alanine and IAA-leucine exhibited similar rooting potential to that of free IBA in Prosopis velutina [Felker & Clark, J Range Manag 1981, 34:466-468]. Van der Krieken et al. [in: Biology of Root Formation and Development, A. Altman and Y. Waisel (eds.), Plenum Press, New York, N.Y., pp. 95-104 (1997)] reported that various IAA and IBA conjugates, mostly amide-linked, proved to be highly active in in vitro root induction in various herbaceous and perennial species compared to the free auxins.

Chloro-substituted phenoxy acid derivatives with auxin activity have long been known. The first phenoxy acids with auxin activity synthesized in 1940 were 2,4-D (2,4-dichlorophenoxyacetic acid) and 2,4,5-TD (2,4,5-trichlorophenoxyacetic acid), characterized as selective herbicides against dicot weeds in cereal and maize fields. In the following years, more phenoxy acid based compounds were examined for their auxin activity, including compounds with phenoxy ring substitutions such as 4-chloro, 2,4-dichloro, 2,4,5-trichloro and 2-methyl-4-chloro, each with three different side chains of acetic, 2-propionic, or 4-butyric acid [Behrens & Morton, Plant Physiol 1963, 38:165-170]. Among such compounds, 2,4-D and MCPA (2-methyl-4-chloro-phenoxyacetic acid) have been used in agriculture as an herbicide [Grossmann, Pest Manag Sci 2010, 66:113-120] and 4-CPA (4-chloro-phenoxyacetic acid) has been used to increase fruit size [Kano, J Hort Sci Biotech 2002, 77:546-550].

Early studies reported that phenoxy acids promote rooting at relatively low concentrations, whereas at high concentrations they are phytotoxic [Weaver, Plant Growth Substances in Agriculture, W.H. Freeman and Co., San Francisco, Calif. (1972)]. Nevertheless, phenoxy acids are generally not used to improve rooting, due to their phytotoxicity.

Tel-Zur [Metabolism of 2-DP and its conjugates in relation to rooting of cuttings, Master Thesis, The Hebrew University of Jerusalem, 1991] reported that a conjugate of 2-DP with glycine methyl ester exhibit high activity in rooting of cuttings of several perennial species, in comparison with IBA. Free 2-DP was released at a rate which differed between the species examined, as determined by application of labeled 2-DP conjugate. It was proposed that slow release of 2-DP from its conjugate might decrease or even eliminate its phytotoxicity.

2,4-D has been reported to undergo conjugation to glutamate and aspartate in plant cells, with the conjugates being reversibly converted to active 2,4-D by hydrolase [Eyer et al., PLoS One 2016, 11:e0159269].

Conjugates of phenoxy acids such as 2,4-D with amines such as 2-amino-4-picoline have been reported to have a strong growth-promoting effect on Arabidopsis hypocotyls, whereas the free phenoxy acids had almost no effect [Savaldi-Goldstein et al., Proc Natl Acad Sci USA 2008, 105:15190-15195]. The higher activity of the conjugates was attributed to their hydrophobic nature, which enabled increased uptake and diffusion to the target tissues.

Additional background art includes Abarca et al. [BMC Plant Biol 2014, 14:354]; Abu-Abied et al. [Plant J 2012, 71:787-799]; Abu-Abied et al. [BMC Genomics 2014, 15:826]; Abu-Abied et al. [PLoS One 2015, 10:e0143828]; Abu-Abied et al. [J Exp Bot 2015, 66:2813-2824]; Blythe et al. [J Environ Hort 2007, 25:166-185]; Dharmasiri et al. [Nature 2005, 435:441-445]; de Almeida et al. [BMC Mol Biol 2010, 11:73]; de Almeida [Plant Sci 2015, 239:155-165]; Diaz-Sala [Front Plant Sci 2014, 5:310]; Hartmann et al. [Hartmann and Kester's Plant Propagation Principles and Practices, Eighth Edition, Pearson Education Limited, Essex, Great Britain (2011)]; Hitchcock & Zimmerman [Contrib Boyce Thomp Inst 1942, 12:497-597]; Legue et al. [Physiol Plant 2014, 151:192-198]; Lipka & Muller [J Exp Bot 2014, 65:4177-4189]; Prigge et al. [G3 (Bethesda) 2016, 6:1383-1390]; Pufky et al. [Funct Integr Genomics 2003, 3:135-143]; Ruedell et al. [Plant Physiol Biochem 2015, 97:11-19]; Sole et al. [Tree Physiol 2008, 28:1629-1639]; Vielba et al. [Tree Physiol 31:1152-1160]; and Vilasboa et al. [Prog Biophys Mol Biol 2018, 50079-6107(18)30228-1].

### SUMMARY OF THE INVENTION

According to an aspect of some embodiments of the invention, there is provided a method of enhancing formation and/or growth of an adventitious root in a plant and/or plant tissue, the method comprising contacting at least a portion of the plant and/or plant tissue with a compound having Formula I:

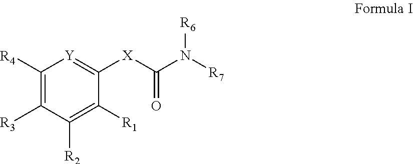

wherein:

X is selected from the group consisting of a bond, CH₂—O—CH₂— and —O—CH₂CH₂CH₂—;

Y is CR₅ or N;

R₁-R₅ are each individually selected from the group consisting of hydrogen, chloro, methyl, methoxy and amino, or alternatively, R₄ and R₅ together form a six-membered aromatic ring;

R₆ is selected from the group consisting of aryl, heteroaryl, alkyl, alkenyl and alkynyl; and

R₇ is selected from the group consisting of hydrogen and alkyl,

or alternatively, R₆ and R₇ together form a five- or six-membered heteroalicyclic ring,

thereby enhancing formation and/or growth of an adventitious root.

According to an aspect of some embodiments of the invention, there is provided a composition for enhancing formation and/or growth of an adventitious root in a plant and/or plant tissue, the composition comprising:

a) a compound having Formula I:

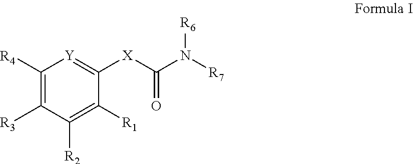

wherein:

X is selected from the group consisting of a bond, —O—CH₂— and —O—CH₂CH₂CH₂—;

Y is CR₅ or N;

R₁-R₅ are each individually selected from the group consisting of hydrogen, chloro, methyl, methoxy and amino;

R₆ is selected from the group consisting of aryl, heteroaryl, alkyl, alkenyl and alkynyl; and

R₇ is selected from the group consisting of hydrogen and alkyl,

or alternatively, R₆ and R₇ together form a five- or six-membered heteroalicyclic ring; and

b) a horticulturally acceptable carrier.

According to an aspect of some embodiments of the invention, there is provided a method of promoting grafting unification, enhancing fruit size and/or of reducing flowering in a plant, the method comprising contacting at least a portion of the plant with a compound having Formula I:

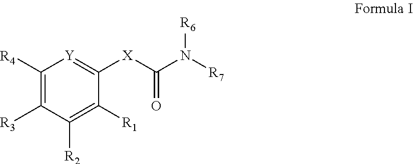

wherein:

X is selected from the group consisting of a bond, CH₂—O—CH₂— and —O—CH₂CH₂CH₂—;

Y is CR₅ or N;

R₁-R₅ are each individually selected from the group consisting of hydrogen, chloro, methyl, methoxy and amino, or alternatively, R₄ and R₅ together form a six-membered aromatic ring;

R₆ is selected from the group consisting of aryl, heteroaryl, alkyl, alkenyl and alkynyl; and

R₇ is selected from the group consisting of hydrogen and alkyl,

or alternatively, R₆ and R₇ together form a five- or six-membered heteroalicyclic ring,

thereby promoting grafting unification, enhancing fruit size and/or reducing flowering.

According to an aspect of some embodiments of the invention, there is provided a composition for promoting grafting unification, enhancing fruit size and/or for reducing flowering in a plant, the composition comprising:

a) a compound having Formula I:

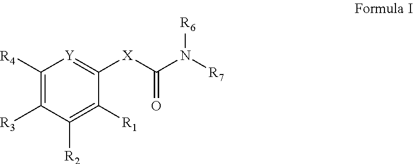

wherein:

X is selected from the group consisting of a bond, —O—CH₂— and —O—CH₂CH₂CH₂—;

Y is CR₅ or N;

R₁-R₅ are each individually selected from the group consisting of hydrogen, chloro, methyl, methoxy and amino;

R₆ is selected from the group consisting of aryl, heteroaryl, alkyl, alkenyl and alkynyl; and

R₇ is selected from the group consisting of hydrogen and alkyl,

or alternatively, R₆ and R₇ together form a five- or six-membered heteroalicyclic ring; and

b) a horticulturally acceptable carrier.

According to an aspect of some embodiments of the invention, there is provided a compound having Formula Ia:

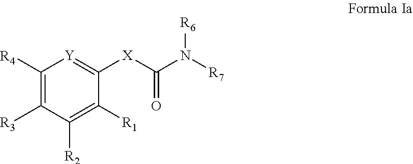

wherein:

X is selected from the group consisting of a bond, CH₂, —O—CH₂— and —O—CH₂CH₂CH₂—;

Y is CR₅ or N;

R₁-R₅ are each individually selected from the group consisting of hydrogen, chloro, methyl, methoxy and amino, or alternatively, R₄ and R₅ together form a six-membered aromatic ring;

R₆ is selected from the group consisting of aryl, alkyl, alkenyl and alkynyl, the alkyl being devoid of a —C(═O)OH substituent at the α-position thereof; and

R₇ is selected from the group consisting of hydrogen and alkyl, wherein when R₇ is alkyl, R₆ is not aryl,

or alternatively, R₆ and R₇ together form a six-membered heteroalicyclic ring.

According to an aspect of some embodiments of the invention, there is provided a method of enhancing formation and/or growth of an adventitious root in a plant and/or plant tissue, the method comprising contacting at least a portion of the plant and/or plant tissue with a compound having Formula Ia (according to any of the respective embodiments described herein), thereby enhancing formation and/or growth of an adventitious root in a plant and/or plant tissue.

According to an aspect of some embodiments of the invention, there is provided a composition for enhancing formation and/or growth of an adventitious root in a plant and/or plant tissue, the composition comprising:

a) a compound having Formula Ia (according to any of the respective embodiments described herein); and

b) a horticulturally acceptable carrier.

According to an aspect of some embodiments of the invention, there is provided a method of promoting grafting unification, enhancing fruit size and/or of reducing flowering in a plant, the method comprising contacting at least a portion of the plant with a compound having Formula Ia (according to any of the respective embodiments described herein), thereby enhancing promoting grafting unification, fruit size and/or of reducing flowering in a plant.

According to an aspect of some embodiments of the invention, there is provided a composition for promoting grafting unification, enhancing fruit size and/or of reducing flowering in a plant, the composition comprising:

a) a compound having Formula Ia (according to any of the respective embodiments described herein); and

b) a horticulturally acceptable carrier.

According to some of any of the embodiments of the invention relating to Formula I and/or Formula Ia, R₁ is selected from the group consisting of hydrogen, chloro and methyl.

According to some of any of the embodiments of the invention relating to Formula I and/or Formula Ia, R₂ is selected from the group consisting of hydrogen and amino.

According to some of any of the embodiments of the invention relating to Formula I and/or Formula Ia, R₃ is selected from the group consisting of hydrogen and chloro.

According to some of any of the embodiments of the invention relating to Formula I and/or Formula Ia, R₃ is chloro.

According to some of any of the embodiments of the invention relating to Formula I and/or Formula Ia, R₁, R₂, R₄ and R₅ are each hydrogen.

According to some of any of the embodiments of the invention relating to Formula I and/or Formula Ia, R₃ is chloro, and R₁, R₂, R₄ and R₅ are each hydrogen.

According to some of any of the embodiments of the invention relating to Formula I and/or Formula Ia, R₄ is selected from the group consisting of hydrogen and chloro.

According to some of any of the embodiments of the invention relating to Formula I and/or Formula Ia, R₅ is selected from the group consisting of hydrogen and methoxy.

According to some of any of the embodiments of the invention relating to Formula I and/or Formula Ia, Y is N.

According to some of any of the embodiments of the invention relating to Formula I and/or Formula Ia, R₁, R₃ and R₄ are each chloro.

According to some of any of the embodiments of the invention relating to Formula I and/or Formula Ia, Y is N, and R₁, R₃ and R₄ are each chloro.

According to some of any of the embodiments of the invention relating to Formula I and/or Formula Ia, X is selected from the group consisting of —O—CH₂— and —O—CH₂CH₂CH₂—.

According to some of any of the embodiments of the invention relating to Formula I and/or Formula Ia, X is a bond.

According to some of any of the embodiments of the invention relating to Formula I and/or Formula Ia, Y is CR₅, R₄ and R₅ together form a six-membered aromatic ring described herein, and X is CH₂.

According to some of any of the embodiments of the invention relating to Formula I and/or Formula Ia, R₇ is hydrogen or methyl.

According to some of any of the embodiments of the invention relating to Formula I and/or Formula Ia, R₆ has Formula II:

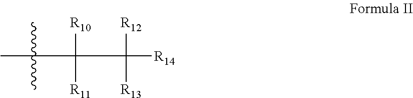

wherein:

R₁₀ and R₁₁ are each selected from the group consisting of hydrogen, alkyl, alkenyl, alkynyl, cycloalkyl, aryl, heteroaryl, heteroalicyclic, carbonyl, thiocarbonyl, C-amido, and C-carboxy; and

R₁₂-R₁₄ are each individually selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of the invention relating to Formula II, R₁₀-R₁₃ are each hydrogen and R₁₄ is hydroxy.

According to some of any of the embodiments of the invention relating to Formula II, R₁₀ is selected from the group consisting of —C(═O)OCH₃, —C(═O)OH or a salt thereof, and —C(═O)NH—(CH₂)₂-R₁₈, wherein R₁₈ is an ionic group.

According to some of any of the embodiments of the invention relating to Formula II, R₁₀ is selected from the group consisting of —C(═O)OCH₃ and —C(═O)NH—(CH₂)₂-R₁₈, wherein R₁₈ is an ionic group.

According to some of any of the embodiments of the invention relating to Formula II:

R₁₀ is —C(═O)OCH₃;

R₁₁ and R₁₂ are each hydrogen; and

R₁₃ and R₁₄ are each —CH₃; or R₁₃ is hydrogen and R₁₄ is indol-3-yl or —C(═O)OCH₃.

According to some of any of the embodiments of the invention relating to a method described herein, the method comprises contacting a base of a plant cutting and at least one leaf of said cutting with a compound having Formula I.

According to some of any of the embodiments of the invention relating to a method described herein, the method further comprises contacting at least a portion of the plant and/or plant tissue with an auxin.

According to some of any of the embodiments of the invention relating to a composition described herein, the composition further comprises an auxin.

According to some of any of the embodiments of the invention relating to an auxin, the auxin comprises indolebutyric acid (IBA).

According to some of any of the embodiments of the invention relating to a carrier, the carrier is selected from the group consisting of talc and an aqueous carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents synthetic auxins (labeled by numbers 1-4) and molecules conjugated thereto (labeled by letters a-t) according to some exemplary embodiments of the invention (only 4-CPA was used for most conjugates in Rounds #2 and #3).

FIGS. 2A-2C present photographs showing rooting of mung bean cuttings upon exposure to 0, 1, 10, 25 or 50 μM of IBA (FIG. 2B) or a conjugate of 2-DP and glycine methyl ester (FIG. 2A), and bar graphs showing the number of roots per cutting upon each treatment (FIG. 2C).

FIG. 3 presents a bar graph showing the number of roots per cutting upon exposure of mung bean cuttings to 2, 10 or 50 μM of free 2-DP (F-2-DP) or 2-DP conjugated to glycine methyl ester (C-2-DP), or exposure to 50 μM of IBA (treatment with water (H₂O) served as a control).

FIGS. 4A-4C present photographs showing rooting of mung bean cuttings upon exposure to 2, 10 or 50 μM of free 4-CPA (F-4-CPA; FIG. 4B) or 4-CPA conjugated to glycine methyl ester (C-4-CPA; FIG. 4A), or exposure to 50 μM of IBA (treatment with water (H₂O) served as a control), and a bar graph showing the number of roots per cutting upon each treatment (FIG. 4C).

FIGS. 5A and 5B present photographs showing rooting of mung bean cuttings upon exposure to 2, 10 or 50 μM of Compound 1o, 1p, 1s or 1t or to 50 μM of IBA (treatment with water (H₂O) served as a control) (FIG. 5A), and a bar graph showing the number of roots per cutting upon each treatment (FIG. 5B); roots were counted after 9 days (different letters above bars indicate statistically significant (p<0.05) difference, as determined by Scheffe analysis).

FIG. 6 presents bar graphs showing the percentage of mature Eucalyptus grandis cuttings which exhibited rooting after submerging (Sub) the cutting base for 1 minute in 100 μM of 4-CPA, MCPA, 2-DP, NAA, and Compounds 1a-4h, or spraying (Spr) with the above compounds (with a surfactant), with or without being submerged for 1 minute in 6000 ppm (28 mM) IBA.

FIG. 7 presents bar graphs showing the percentage of mature Eucalyptus grandis cuttings which exhibited callus formation after submerging (Sub) the cutting base for 1 minute in 100 μM of 4-CPA, MCPA, 2-DP, NAA, and Compounds 1a-4h and/or by spraying (Spr) the foliage with the aforementioned compounds (with a surfactant), with or without being submerged for 1 minute in 6000 ppm IBA (rooting percentage was recorded after 45 days).

FIG. 8 presents a graph showing percent rooting induced by a conjugate in the presence of IBA (as described for FIG. 6) as a function of the pKA of the amine used to prepare the conjugate.

FIGS. 9A and 9B present a bar graph showing the percentage of mature Eucalyptus grandis cuttings which exhibit rooting following treatment of the cutting base with 100 μM of 4-CPA or any one of Compounds 1i, 1j, 1k, 1l, 1m and 1n by submerging (sub) the cutting base for 1 minute and/or by spraying (spr) the foliage, with (FIG. 9B) or without (FIG. 9A) treatment with 6000 ppm IBA by submersion for 1 minute (each treatment included 3 replicates, 20-25 cuttings each (total of 60-75), rooting percentage was scored after 45-60 days).

FIGS. 10A and 10B present a bar graph showing the percentage of mature Eucalyptus grandis cuttings which exhibit rooting following treatment of the cutting base with 100 μM of 4-CPA, 4-CPA glycine methyl ester conjugate (4-CPA-Gly), or any one of Compounds 1o, 1p, 1q, 1r, 1s and 1t by submerging (right bars) the cutting base for 1 minute and/or by spraying (left bars) the foliage, with (FIG. 10B) or without (FIG. 10A) treatment with 6000 ppm IBA by submersion for 1 minute (each treatment was applied to 20-25 cuttings in 3 repeats (total of 60-75), rooting percentage was scored after 45-60 days; * indicates p<0.05 relative to IBA only treatment, as determined by Scheffe analysis).

FIG. 11 presents a bar graph showing the percentage of adventitious root formation upon treating Eucalyptus grandis cuttings with 6000 ppm IBA (by submersion of the cutting base) alone or in combination with 100 μM of Compound 1o, 1p, 1s or 1t or 4-CPA by both submersion of the cutting base and spraying of foliage (each treatment was applied to 20 cuttings in 3 repeats; * indicates p<0.05 relative to IBA only treatment, as determined by Scheffe analysis).

FIG. 12 presents photographs showing representative Eucalyptus grandis cuttings treated with 6000 ppm IBA (by submersion) alone or in combination with 100 μM of Compound 1o, 1p, 1s or 1t or 4-CPA by both submersion and spraying, as described for FIG. 11.

FIGS. 13A and 13B present bar graphs showing total root length for roots with various diameter ranges (FIG. 13A) and number of tips of roots with a diameter of 0-0.5 mm (left bars) or 0.5-1 mm (small right bars) (FIG. 13B) for Eucalyptus grandis cuttings treated with IBA alone or in combination with 100 μM of Compound 1o, 1p, 1s or 1t or 4-CPA by both submersion and spraying (each treatment was applied to 20 cuttings; * indicates p<0.05 relative to IBA only treatment, as determined by Scheffe analysis).

FIGS. 14A and 14B present fluorescent microscopy images (FIG. 14A) and a bar graph (FIG. 14B) showing fluorescence 4 hours (left bars in FIG. 14B) or 27 hours (right bars in FIG. 14B) after Arabidopsis plants expressing DR5-venus were transferred to plates with 10 μM of IBA, 4-CPA or any one of Compounds 1o, 1p, 1s and 1t (MS medium served as a control); different letters above bars (small letters for 4 hours and capital letters for 27 hours) show statistically difference (p<0.05) by T-test.

FIGS. 15A and 15B present photographic images (FIG. 15A) showing representative examples after 5 days, and a bar graph (FIG. 15B) showing root length (as percentage of initial length) as a function of time, in four day old Arabidopsis seedlings transferred to vertical plates containing 10 nM, 50 nM, 100 nM, 1 μM or 10 μM of IBA or 4-CPA, for 5 days (for each treatment, two plates were examined including 20 seedlings; MS medium served as a control).

FIGS. 16A and 16B present photographic images (FIG. 16A) showing representative examples, and a bar graph (FIG. 16B) showing root length (as percentage of initial length) in Arabidopsis seedlings transferred for 5 days to vertical plates containing 50 nM of IBA, 4-CPA or any one of Compounds 1o-1t (MS medium served as a control).

FIGS. 17A and 17B present photographic images (FIG. 17A) showing adventitious root formation, and a bar graph (FIG. 17B) showing adventitious root (right bars) and lateral root (left bars) formation, in 5 day-old etiolated intact Arabidopsis seedlings incubated for one hour in 10 μM of 4-CPA or any one of Compounds 1o, 1p, and 1t, and then grown in vertical plates kept in the dark for 5 days (MS medium served as a control, scale bar=2 mm); different letters above bars (small letters for lateral roots and capital letters for adventitious roots) show statistically difference (p<0.05) by T-test.

FIG. 18 presents a bar graph showing adventitious root formation in 4 day-old etiolated intact Arabidopsis seedlings incubated for one hour in 10 μM of 4-CPA or conjugates of 4-CPA with L-Phe, D-Phe, L-Met, D-Met, L-Glu, D-Glu, L-Trp or D-Trp, and then grown in vertical plates kept in the dark for 5 days (MS medium served as a control).

FIGS. 19A and 19B present a photograph (FIG. 19A) of representative rooted argan cutlings treated with IBA and Compound 1t, and a bar graph (FIG. 19B) showing rooting in argan cuttings treated with IBA alone or in combination with Compound 1s or 1t (* indicates P<0.05 relative to IBA only treatment, as determined by Scheffe analysis).

FIGS. 20A and 20B present a photograph (FIG. 20A) and bar graph (FIG. 20B) showing rooting in jojoba cuttings exposed to a commercial (T-8) rooting treatment or to Compounds 1o-1t.

FIG. 21 presents a bar graph showing the percentage of etiolated (51W) or green (51) branches of vc51 avocado rootstock following treatment with IBA alone or IBA with Compound 1l, 1s, 2h, 3g, 3f or 4b.

FIGS. 22A-22I present images of representative etiolated (FIGS. 22H and 22I) or green (FIGS. 22A-22G) branches of vc51 avocado rootstock following treatment with IBA alone (FIGS. 22A and 22H) or IBA with Compound 2h (FIG. 22B), 4b (FIGS. 22C and 22I), 3g (FIG. 22D), 3f (FIG. 22E), 11 (FIG. 22F) or 1s (FIG. 22G).

FIG. 23 presents a bar graph showing the average number of roots per cutting, for etiolated (51W) or green (51) cuttings of vc51 avocado rootstock, following treatment with IBA 15 alone or IBA with Compound 1l, 1s, 2h, 3g, 3f or 4b.

FIGS. 24A-24D present micrographic images of a callus formed upon exemplary treatment of avocado cuttings, showing circular cell wall thickening (FIG. 24A), cork layer (FIG. 24B), and amyloplasts (FIGS. 24C and 24D; FIG. 24D represents image under polarized light) (pertinent features indicated by arrows).

FIGS. 25A-25H presents bar graphs showing rooting (left bars) and callus-formation (right bars) rates (FIGS. 25A and 25E), mean root number per cutting (FIGS. 25B and 25F), and mean root length per cutting (FIGS. 25C and 25G), and images of representative cuttings (FIGS. 25D and 25H), upon rooting of cuttings from E. brachyphylla (FIGS. 25A-25D) and E. x trabutii (FIGS. 25E-25H) in the presence of 6000 ppm IBA alone or in combination with Compound 1s or 1t (a.k.a. “52” and “53”, respectively); bars represent averages of 3 repeats (* indicates p<0.05, ** indicates p<0.01).

FIG. 26 presents a schematic depiction of an assay in which 5 day-old etiolated Arabidopsis seedlings were incubated for 24 hours on a split petri dish with MS media supplemented with 10 μM of the tested compound, with the shoot placed on one half of the plate and the root exposed to the other half; after 24 hours the seedlings were transferred to MS plates without tested compound.

FIG. 27 presents images of two representative Arabidopsis seedlings (via stereo microscope) in which the shoot and root were each treated independently with 4-CPA, Compound 1p or Compound 1t, or with MS medium.

FIG. 28 presents a bar graph showing mean root length in Arabidopsis seedlings in which the shoot and root were each treated independently with 4-CPA, Compound 1p or Compound 1t, or with MS medium (n=10; * indicates p<0.05, ** indicates p<0.01, and *** indicates p<0.001 relative to control, as determined by Tukey-Kremer multiple comparisons; groups with different letters are significantly different from each other (p<0.05); shoot treatment is indicated prior to root treatment, e.g., “4-CPA/MS” indicates that shoot was treated with 4-CPA and root with MS).

FIG. 29 presents a bar graph showing mean number of lateral roots in Arabidopsis seedlings in which the shoot and root were each treated independently with 4-CPA, Compound 1p or Compound 1t, or with MS medium (n=10; ** indicates p<0.01, and *** indicates p<0.001 relative to control, as determined by Steel-Dwass multiple comparisons on log transform data; groups with different letters are significantly different from each other (p<0.05); shoot treatment is indicated prior to root treatment, e.g., “4-CPA/MS” indicates that shoot was treated with 4-CPA and root with MS).

FIG. 30 presents a bar graph showing mean number of adventitious roots in Arabidopsis seedlings in which the shoot and root were each treated independently with 4-CPA, Compound 1p or Compound 1t, or with MS medium (n=10; *** indicates p<0.001 relative to control, as determined by Steel-Dwass multiple comparisons on log transform data; groups with different letters are significantly different from each other (p<0.05); shoot treatment is indicated prior to root treatment, e.g., “4-CPA/MS” indicates that shoot was treated with 4-CPA and root with MS).

FIGS. 31A and 31B present bar graphs showing basal (FIG. 31A) and foliar (FIG. 31B) 4-CPA levels (as determined by LC-MS) in mature Eucalyptus grandis cuttings 0, 1, 6, 24 and 216 hours after treatment of the cuttings with 4-CPA by base submersion (sub) or by spraying the foliage (spr); untreated cuttings used as control (* indicates p<0.05 relative to control by T-test).

FIGS. 32A and 32B present bar graphs showing basal (FIG. 32A) and foliar (FIG. 32B) 4-CPA levels (as determined by LC-MS) in mature Eucalyptus grandis cuttings 0, 6, 24 and 48 hours after treatment of the cuttings with IBA alone or with Compound 1s or 1t (* indicates p<0.05 relative to control by T-test).

FIGS. 33A and 33B present bar graphs showing basal (FIG. 33A) and foliar (FIG. 33B) levels of indoleacetic acid (IAA) (as determined by LC-MS) in mature Eucalyptus grandis cuttings 0, 6, 24 and 48 hours after treatment of the cuttings with IBA alone or with Compound 1t (* indicates p<0.05 relative to control by T-test).

FIGS. 34A and 34B present bar graphs showing basal (FIG. 34A) and foliar (FIG. 34B) levels of IBA (as determined by LC-MS) in mature Eucalyptus grandis cuttings 0, 6, 24 and 48 hours after treatment of the cuttings with IBA alone or with Compound 1t (* indicates p<0.05 relative to control by T-test).

FIGS. 35A and 35B present bar graphs showing basal (FIG. 35A) and foliar (FIG. 35B) levels of IAA-aspartate conjugate (as determined by LC-MS) in mature Eucalyptus grandis cuttings 0, 6, 24 and 48 hours after treatment of the cuttings with IBA alone or with Compound 1t (* indicates p<0.05 relative to control by T-test).

FIGS. 36A and 36B present bar graphs showing basal (FIG. 36A) and foliar (FIG. 36B) levels of 2-oxindole-3-acetic acid (OxIAA) (as determined by LC-MS) in mature Eucalyptus grandis cuttings 0, 6, 24 and 48 hours after treatment of the cuttings with IBA alone or with Compound 1t (* indicates p<0.05 relative to control by T-test).

FIGS. 37A and 37B present bar graphs showing basal (FIG. 37A) and foliar (FIG. 37B) levels of IAA-glutamate conjugate (as determined by LC-MS) in mature Eucalyptus grandis cuttings 0, 6, 24 and 48 hours after treatment of the cuttings with IBA alone or with Compound 1t.

FIGS. 38A and 38B present bar graphs showing basal (FIG. 38A) and foliar (FIG. 38B) levels of IBA-aspartate conjugate (as determined by LC-MS) in mature Eucalyptus grandis cuttings 0, 6, 24 and 48 hours after treatment of the cuttings with IBA alone or with Compound 1t (* indicates p<0.05 relative to control by T-test).

FIGS. 39A-39C present images of a eucalyptus cutting base section (FIG. 39A), inner part after peeling the bark (FIG. 39B) and the part of the bark containing cambium (FIG. 39C), which were used to extract RNA from cambium enriched-fractions of cells scraped from the peeled bark according to some embodiments of the invention.

FIGS. 40A and 40B present bar graphs showing real time PCT using specific markers WOX4 (FIG. 40A) and HB8 (FIG. 40B) to ensure cambium cell enrichment according to some embodiments of the invention.

FIG. 41 presents a table showing the transcripts relating to cytokinin which are expressed differently between treatment with IBA and absence of treatment (0), or between treatment with IBA and treatment with IBA and Compound 1t.

FIG. 42 presents a table showing the transcripts relating to the cell wall which are expressed differently between treatment with IBA and absence of treatment (0), or between treatment with IBA and treatment with IBA and Compound 1t.

FIG. 43 presents a table showing the transcripts relating to the cell division and meristematic cells, which are expressed differently between treatment with IBA and absence of treatment (0), or between treatment with IBA and treatment with IBA and Compound 1t.

FIGS. 44A and 44B present photographic images (FIG. 44A) of representative DR5-Venus-expressing Arabidopsis plants exposed to 10 μM of IBA, 4-CPA or 4-CPA conjugates in the form of a methyl ester (4-CPA-L-Val, 4-CPA-L-Asp, or 4-CPA-L-Trp) or a sodium salt (Na-4-CPA-L-Val, Na-4-CPA-L-Asp, or Na-4-CPA-L-Trp), and a bar graph (FIG. 44B) showing the DR5 fluorescence levels in plants exposed to the aforementioned treatments (“CPA”=4-CPA, 1p=4-CPA-L-Val, 1r=4-CPA-L-Asp, 1t=4-CPA-L-Trp, 83=4-CPA-L-Val sodium salt, 84=4-CPA-L-Asp disodium salt, 82=4-CPA-L-Trp sodium salt; MS medium served as a control).

FIGS. 45A and 45B present bar graphs showing percentage of rooting (FIG. 45A) and number of roots (FIG. 45B) in a cannabis clone treated for 1 minute with 6000 ppm IBA alone or in combination with 50 μM of Compound 82 (* indicates p<0.05, as determined by Scheffe analysis) FIG. 46 presents a bar graph showing mean number of adventitious roots in etiolated Arabidopsis seedlings incubated for 1 hour with 10 μM of the indicated 4-CPA-amino acid conjugates or 4-CPA (“49”=Compound 1p (4-CPA-L-Val-ester), “52”=Compound 1s (4-CPA-D-Trpl-ester), “53”=Compound 1t (4-CPA-L-Trp-ester), Compound 82=(4-CPA-L-Val sodium salt); MS medium served as a control).

FIG. 47 presents a schematic depiction of a synthesis of conjugates according to some embodiments of the invention.

### DESCRIPTION OF SPECIFIC EMBODIMENTS OF THE INVENTION

The present invention, in some embodiments thereof, relates to treatment of plants, and more particularly, but not exclusively, to compounds useful for inducing root formation in plants, such as in plant cuttings, and for promoting grafting unification, enhancing fruit size and reducing flowering.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that carboxylic acids which exhibit toxic auxin activity towards plants may surprisingly be converted to compounds which can effectively enhance rooting in plants (without substantial toxicity) by conjugation with an amine to form an amide. It was further uncovered that modulation of the toxicity and rooting enhancement may be modulated by selection of appropriate amines for conjugation. While reducing the present invention to practice, the inventors have prepared various conjugates which enhance rooting in cuttings taken even from plants which are known to be very difficult to root from cuttings, and studied the relationship between amine structure and modulation of toxicity and rooting enhancement.

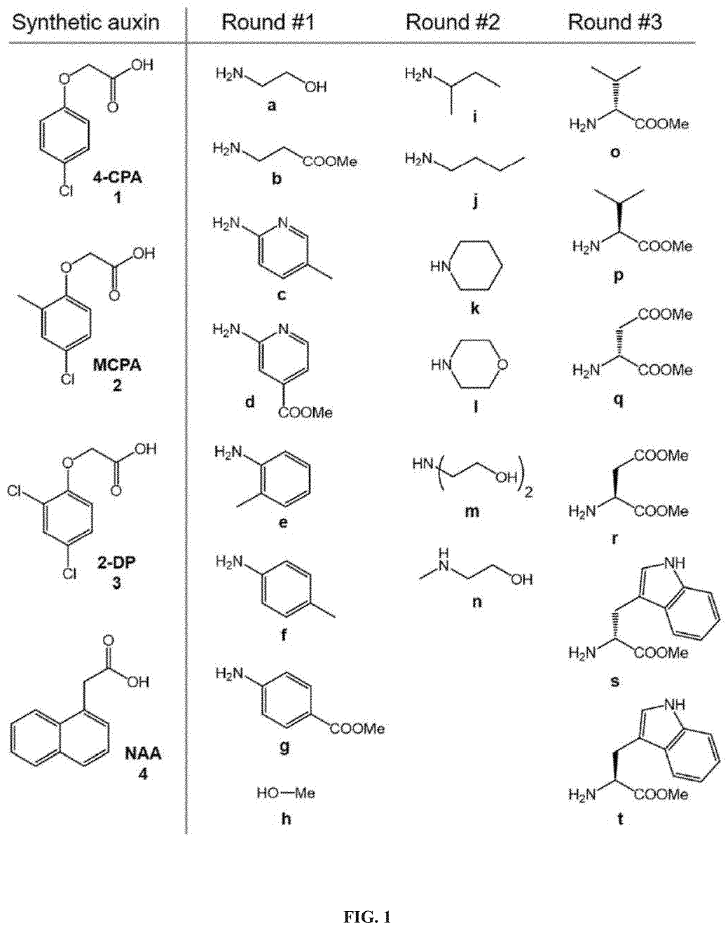

Referring now to the drawings, FIG. 1 depicts compounds used to prepare exemplary conjugates.

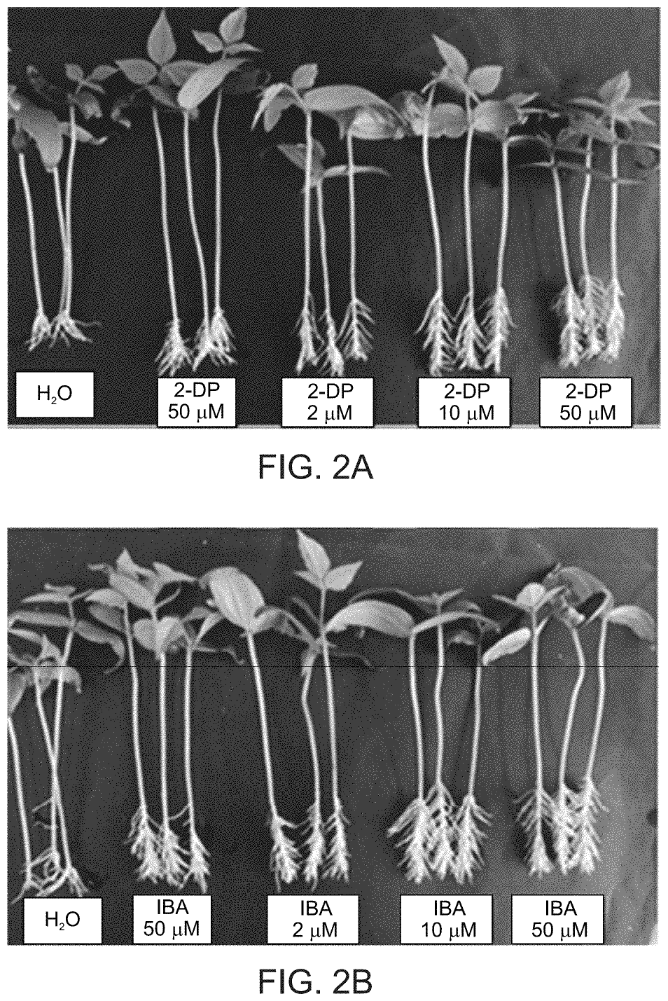

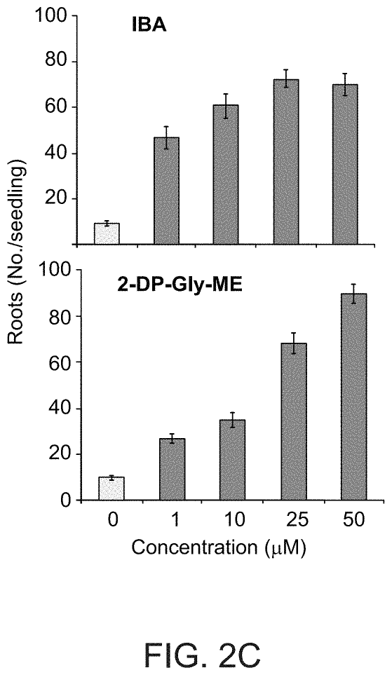

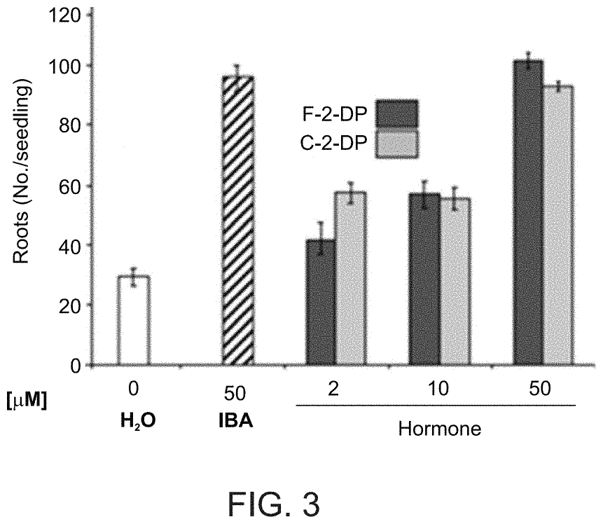

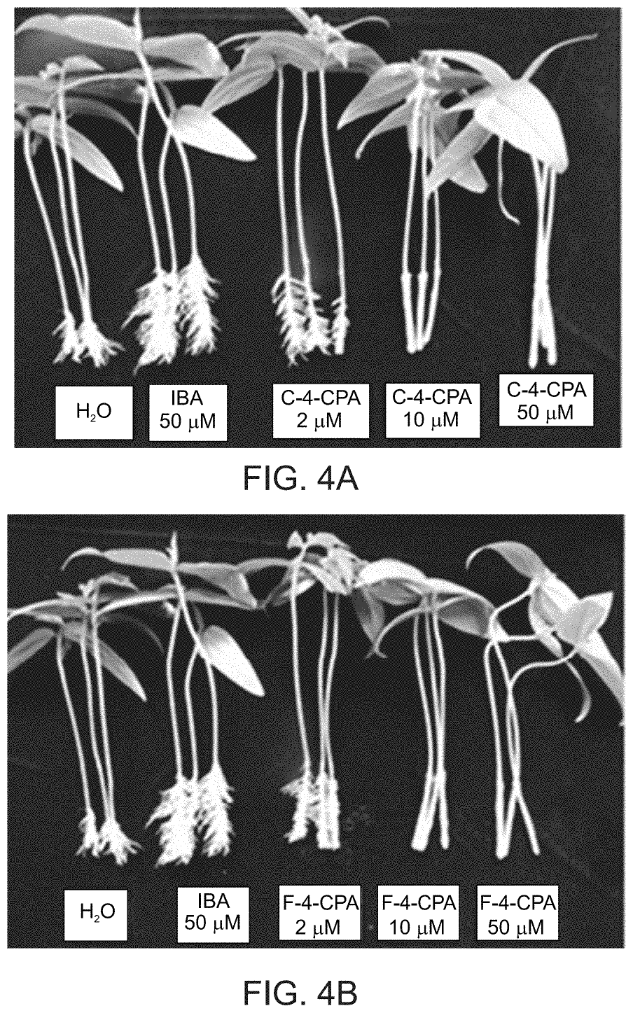

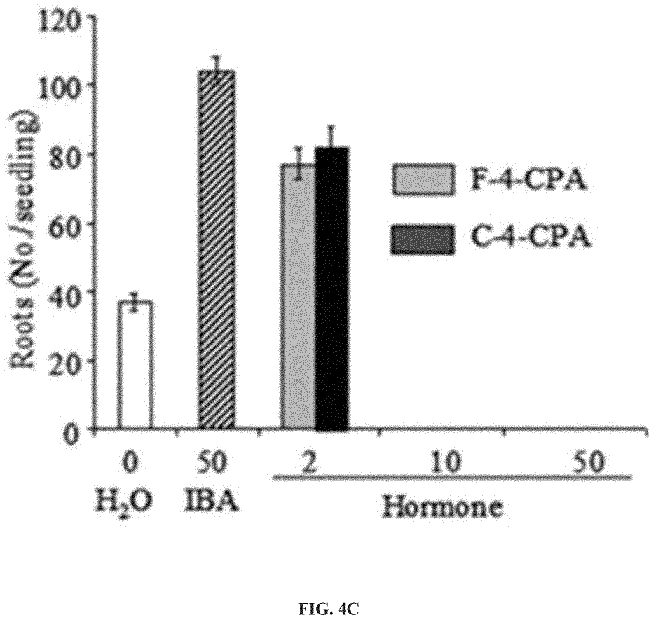

FIGS. 2A-3 shows that 2-DP and the conjugate thereof with glycine methyl ester enhance root formation in a mung bean model. FIGS. 4A-4C show that 4-CPA and the conjugate thereof with glycine methyl ester inhibit adventitious root formation in a mung bean model, but enhance root formation at a low concentration.

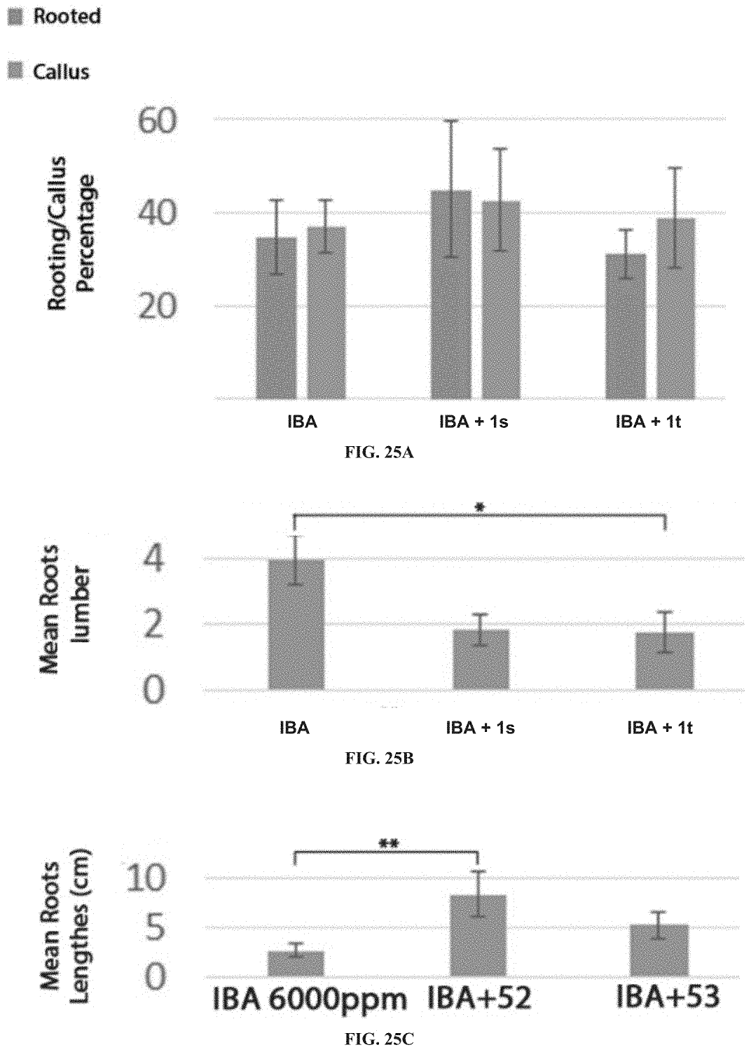

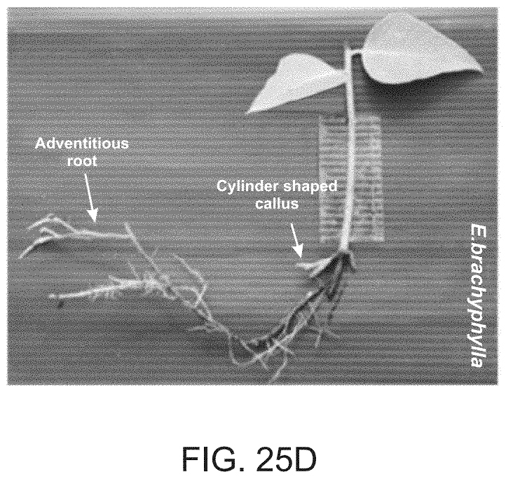

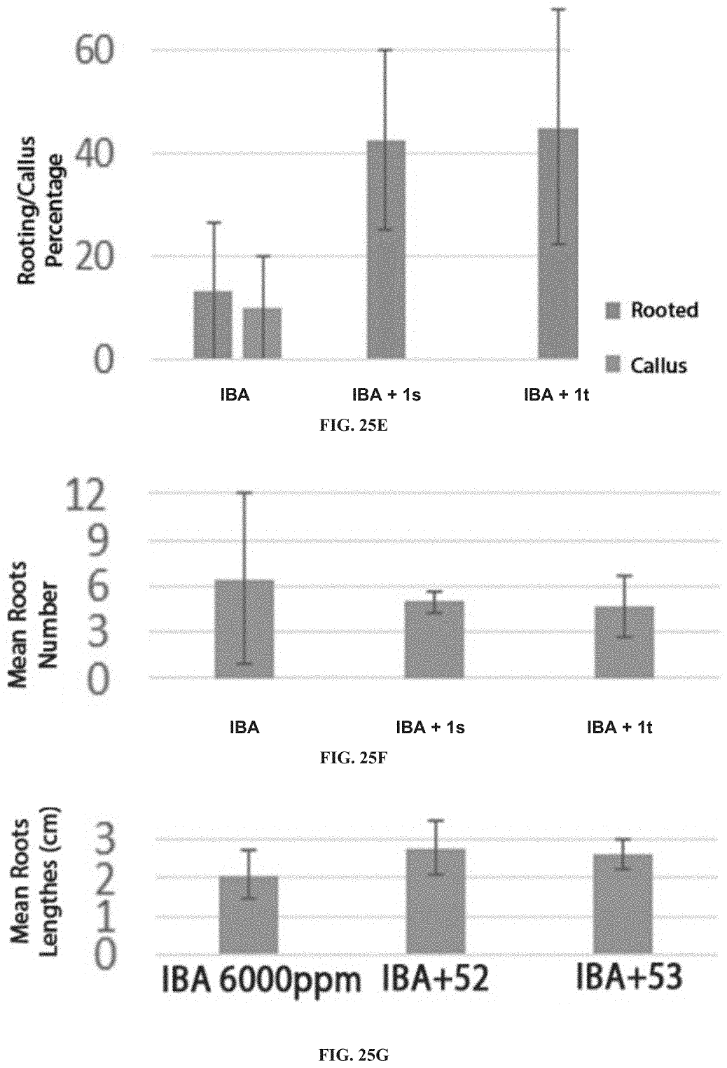

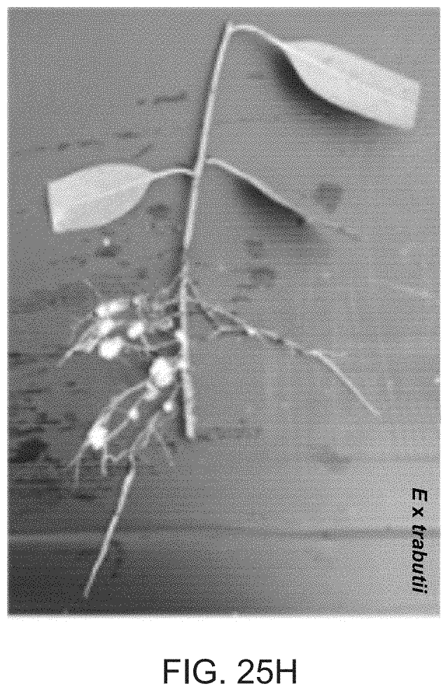

FIGS. 6-13B and 46 show that exemplary conjugates can enhance root formation in Eucalyptus grandis cuttings, a model in which root formation is difficult to induce, and that resistance to hydrolysis is not associated with enhanced root formation in this model. FIGS. 25A-25H show that exemplary conjugates can enhance the rooting percentage or rate of root formation in cuttings of other eucalyptus species.

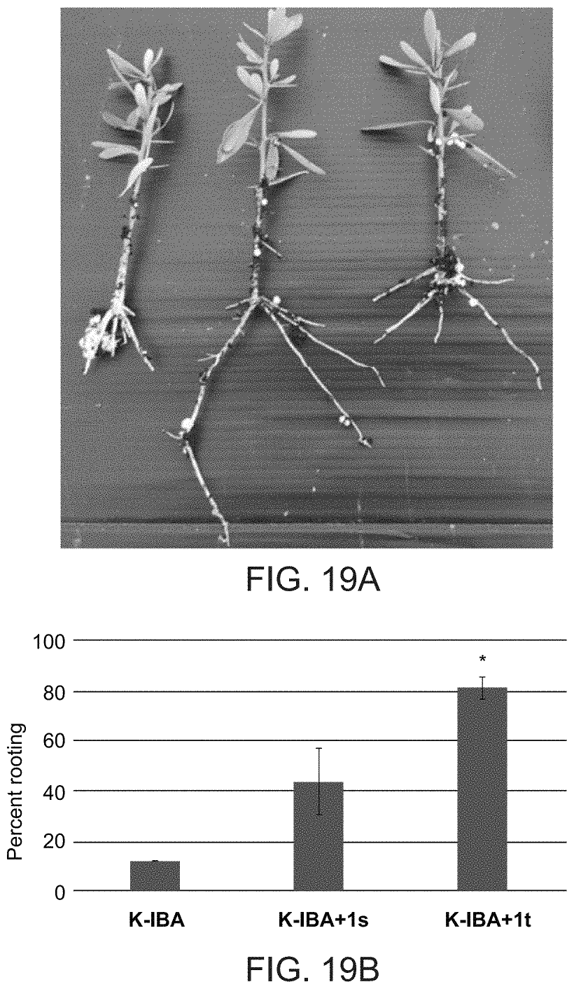

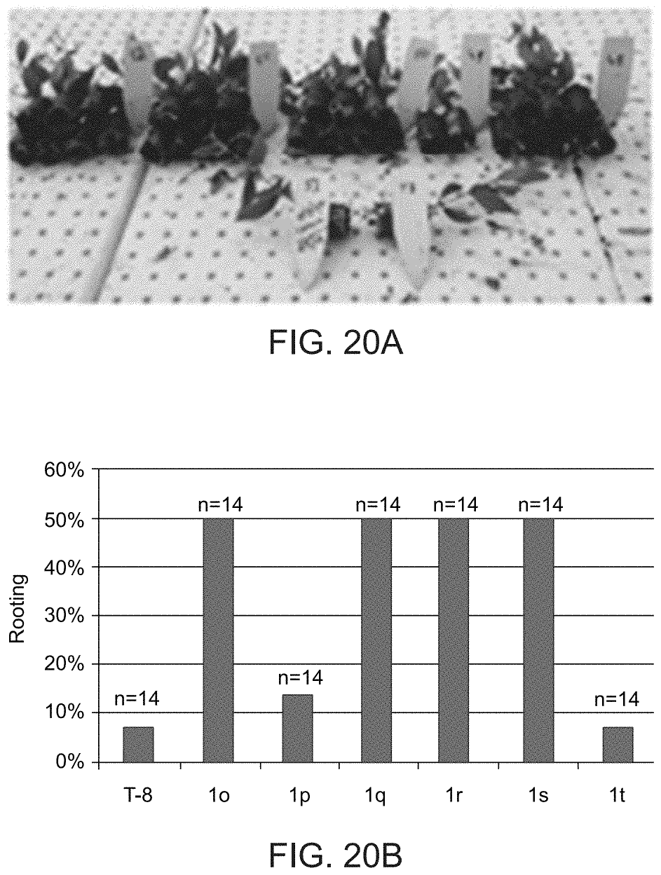

FIGS. 19A-20B show that exemplary conjugates can enhance root formation in argan and jojoba cuttings.

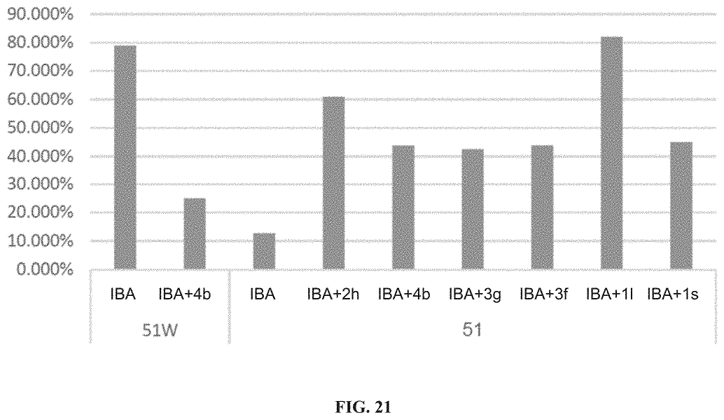

FIGS. 21-23 show that in avocado cuttings (a difficult to root model), etiolated branches root more effectively than do green branches in samples treated only with IBA, whereas in samples treated with exemplary conjugates (in addition to IBA), root formation in green branches was enhanced even to the point of being more effective than root formation in etiolated branches.

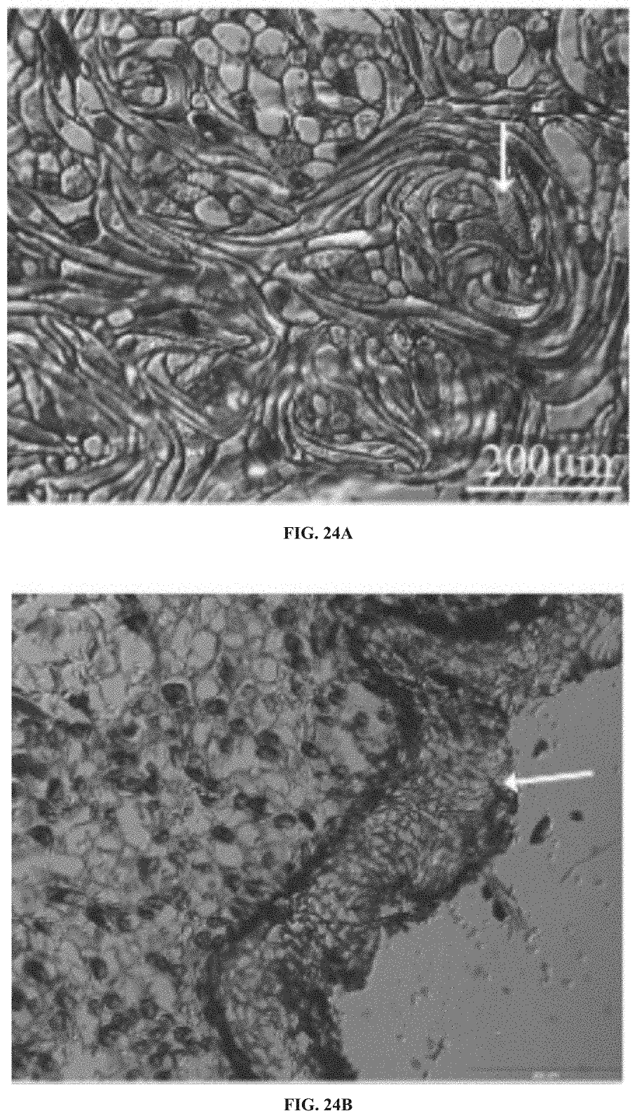

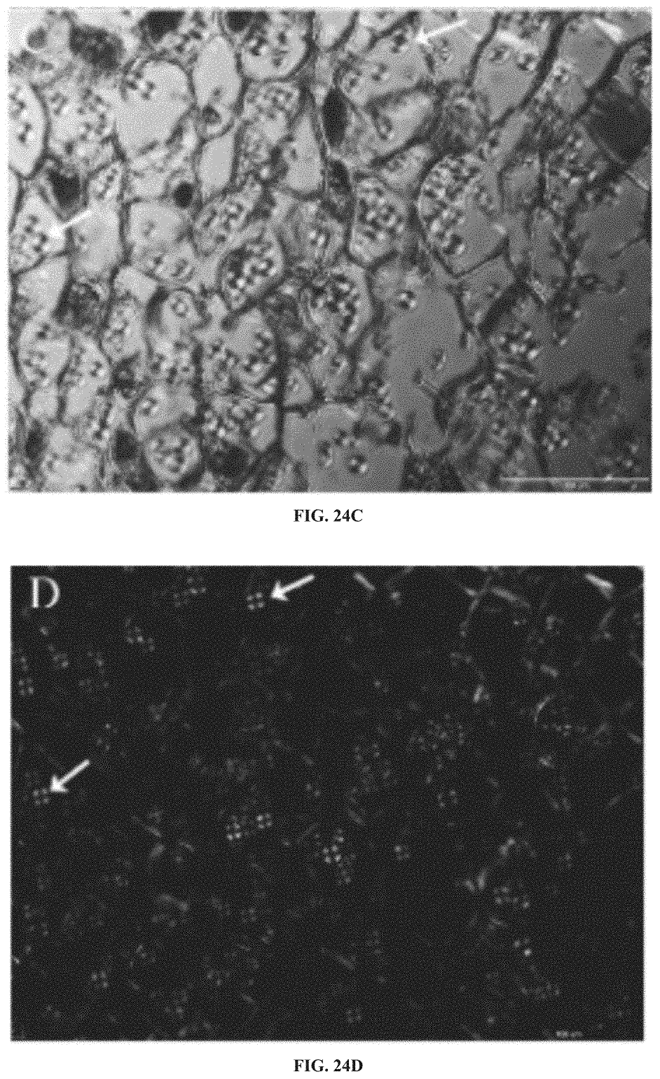

FIGS. 24A-24D show that roots originate from the callus which develops at the base of avocado cuttings.

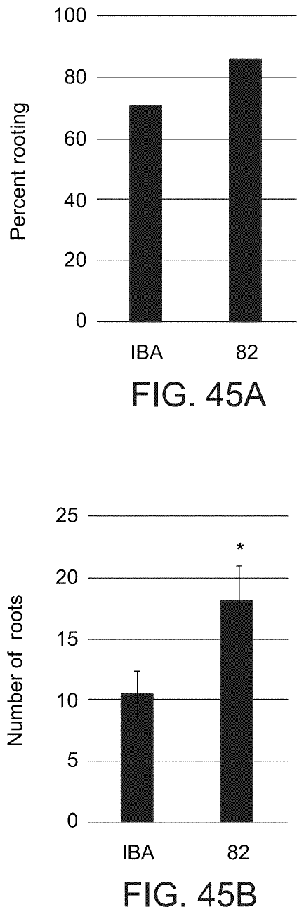

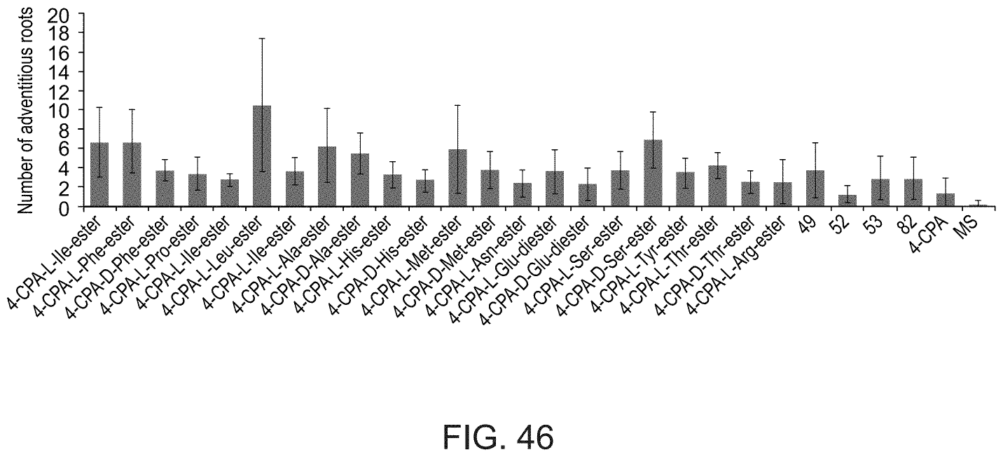

FIGS. 45A and 45B show that exemplary conjugates can enhance root formation in cannabis.

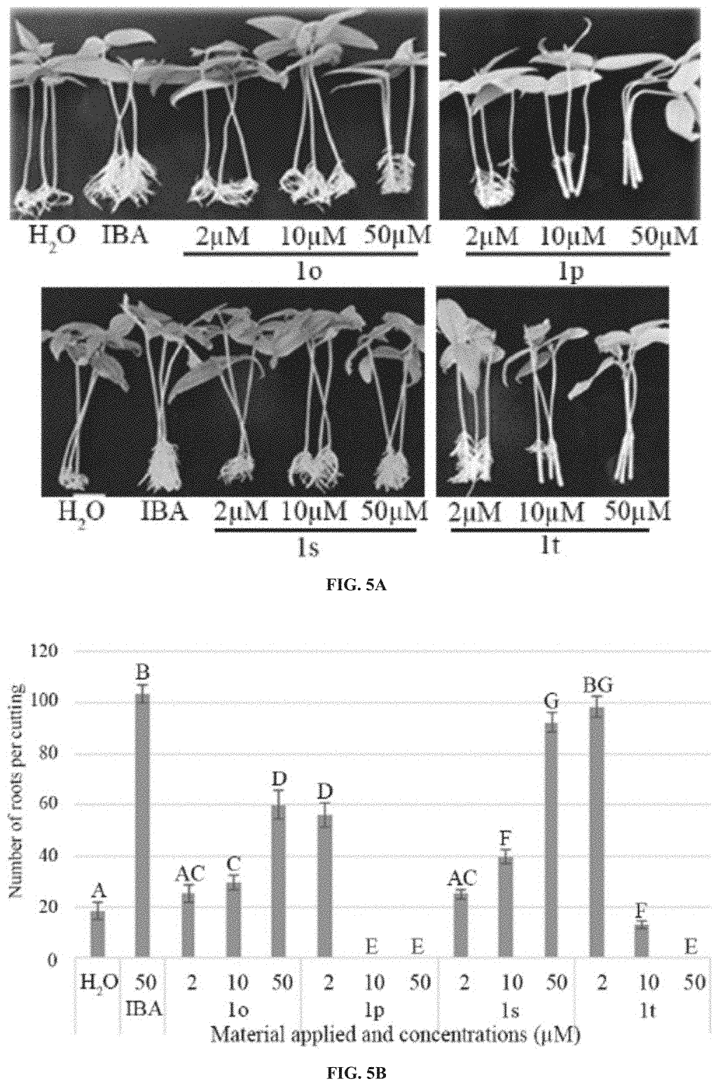

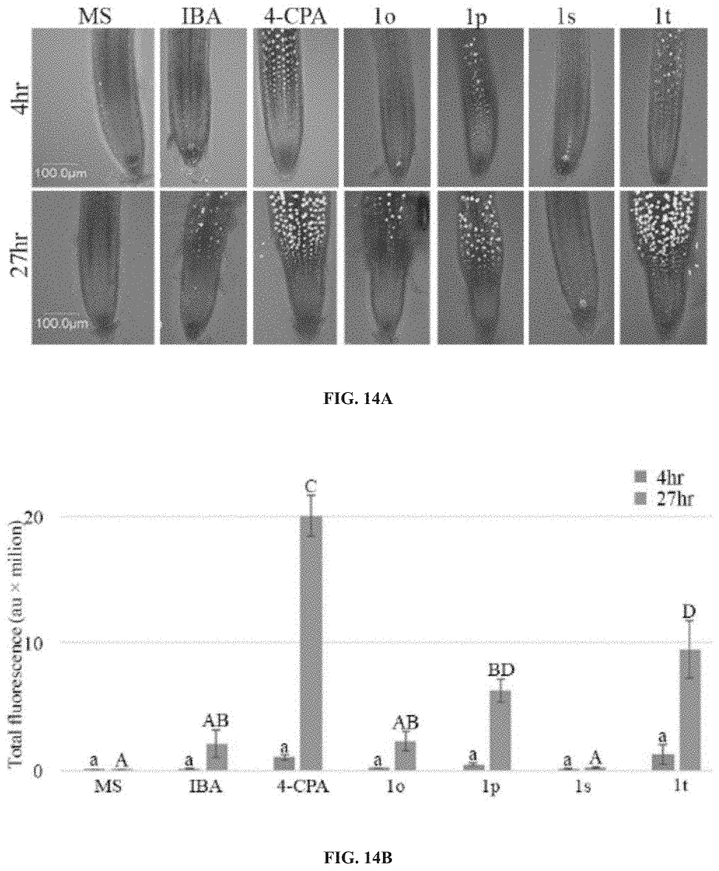

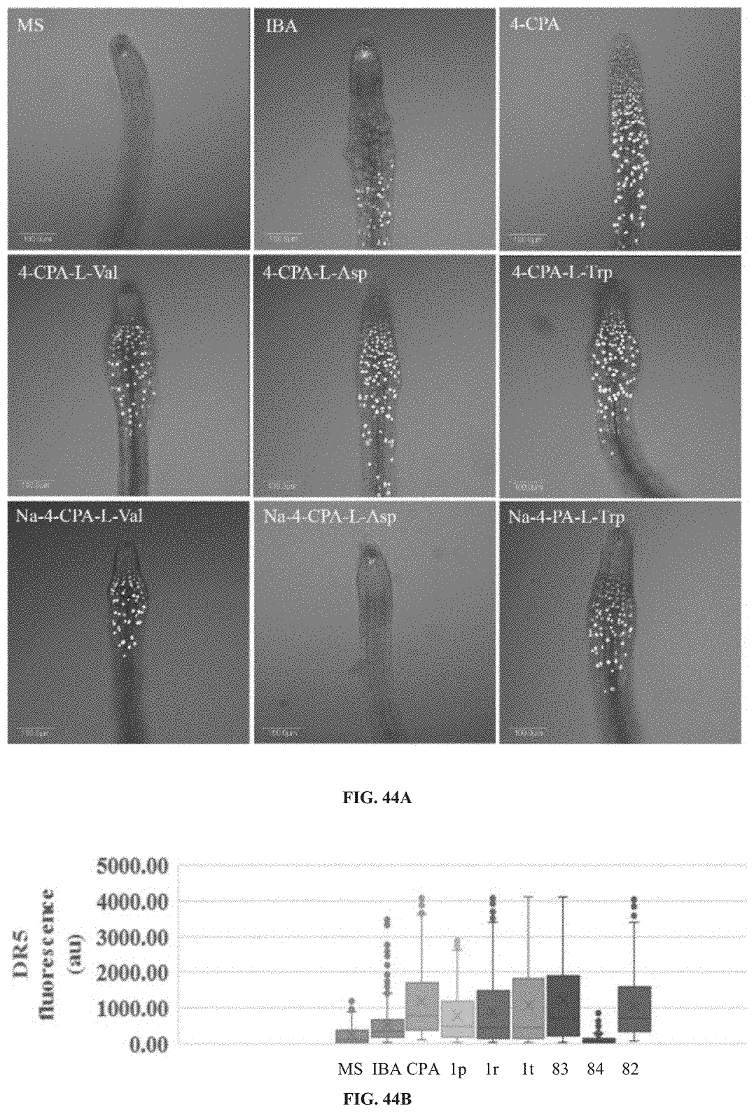

FIGS. 5A-5B and 14A-18 show that conjugates of 4-CPA with L-amino acids exhibit more potent auxin activity, in a mung bean model (FIGS. 5A and 5B) and in an Arabidopsis model (FIGS. 14A-18), than do conjugates of 4-CPA with D-amino acids (and less potent auxin activity than free 4-CPA), indicating that rate of hydrolysis is associated with the degree of auxin activity. FIGS. 44A and 44B show that most conjugates of 4-CPA with (non-esterified) amino acid sodium salts exhibit comparable activity to that of conjugates of 4-CPA with amino acid methyl esters.

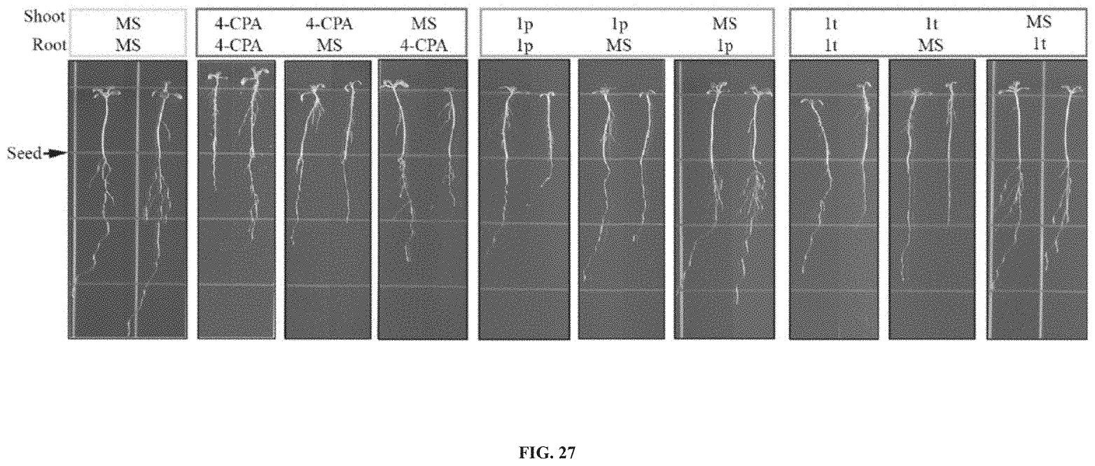

FIGS. 27-38B show that application of 4-CPA conjugates (or 4-CPA) to leaves results in highly effective translocation of 4-CPA from the leaves to the site of root formation.

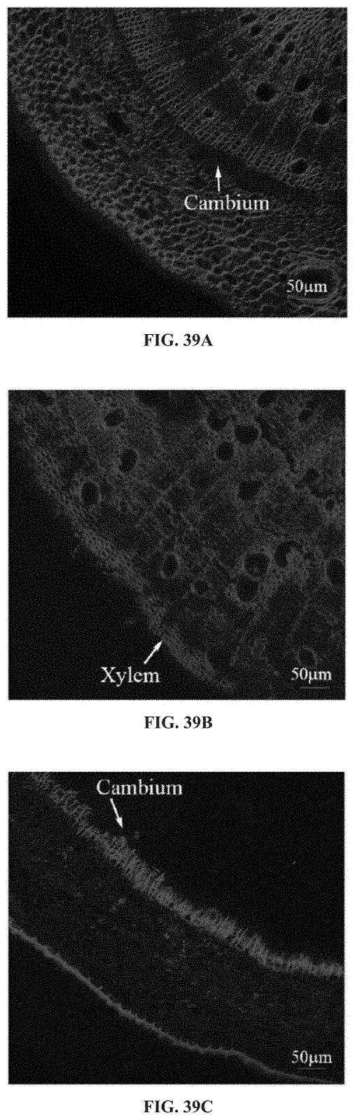

FIGS. 39A-43 show that an exemplary conjugate alters gene expression in cambium cells, which may explain, e.g., the promotion of root formation.

Embodiments of the present invention therefore generally relate to newly designed compounds and to uses thereof, e.g., in enhancing rooting in a plant and/or plant tissue.

Compound:

The compounds according to some of the present embodiments are collectively represented by Formula I:

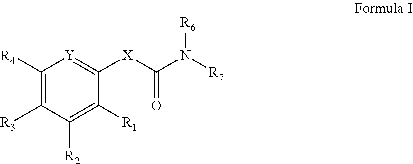

wherein:

X is a bond, CH₂—O—CH₂— or —O—CH₂CH₂CH₂—;

Y is CR₅ or N;

R₁-R₅ are each hydrogen, chloro, methyl, methoxy and/or amino, or alternatively, R₄ and R₅ together form a six-membered aromatic ring;

R₆ is aryl, heteroaryl, alkyl, alkenyl or alkynyl; and

R₇ is hydrogen or alkyl, or alternatively, R₆ and R₇ together form a five- or six-membered heteroalicyclic ring.

Compound of Formula I may optionally be described as a conjugate of an amine (having the formula HNR₆R₇, wherein R₆ and R₇ are as defined in Formula I) and a carboxylic acid and/or as being composed of an amino moiety (having the formula —NR₆R₇, wherein R₆ and R₇ are as defined in Formula I) and an acyl moiety.

In some of any of the respective embodiments, the abovementioned amine (as defined by R₆ and R₇) is characterized by a pKa of at least 8.0, and optionally at least 8.5, or at least 9.0, or at least 9.5. Examples of of amines having such a pKa include, without limitation, most primary alkylamines (wherein R₇ is hydrogen and R₆ is alkyl).

In some of any of the respective embodiments, the abovementioned amine is characterized by a pKa of no more than 11.0, for example, in a range of from 8.0 to 11.0, or from 8.5 to 11.0, or from 9.0 to 11.0 or from 9.5 to 11.0. In some embodiments, the pKa is no more than 10.5, for example, in a range of from 8.0 to 10.5, or from 8.5 to 10.5, or from 9.0 to 10.5 or from 9.5 to 10.5. In some exemplary embodiments, the pKa is about 9.6.

Without being bound by any particular theory, it is believed that a relatively low pKa is associated by higher lability (of the amide bond of Formula I) and that a relatively high pKa is associated by lower lability, and that pKa values in a range described herein result in a desirable degree of lability.

Exemplary compounds according to Formula I are described in the Examples section herein, as well as processes by which such compounds may optionally be prepared by conjugating the appropriate acid and amine.

In some of any of the respective embodiments, R₁ is hydrogen, halo or alkyl (e.g., C₁₋₄-alkyl). In some such embodiments, the halo is chloro and/or the alkyl is methyl. In some embodiments, R₁ is hydrogen.

In some of any of the respective embodiments, R₂ is hydrogen or amino (e.g., —NH₂). In some embodiments, R₂ is hydrogen. In some embodiments, R₁ and R₂ are both hydrogen.

In some of any of the respective embodiments, R₁ is hydrogen, halo or alkyl (e.g., C₁₋₄-alkyl) according to any of the respective embodiments described herein, and R₂ is hydrogen or amino (e.g., —NH₂) according to any of the respective embodiments described herein.

In some of any of the respective embodiments, R₃ is hydrogen or halo, optionally hydrogen or chloro. In some embodiments, R₃ is chloro. In some such embodiments, R₃ is chloro and R₁ is hydrogen, halo (e.g., chloro) or alkyl (e.g., methyl). 4-Chlorophenoxyacetyl, 4-chloro-2-methylphenoxyacetyl, 2,4-dichlorophenoxyacetyl, 2,4,5-trichlorophenoxyacetyl, 4-(4-chlorophenoxy)butanoyl, 4-(4-chloro-2-methylphenoxy)butanoyl, 4-(2,4-dichlorophenoxy)butanoyl, 4-(2,4,5-trichlorophenoxy)butanoyl, 3,5,6-trichloro-2-pyridinyloxyacetyl, and 4-amino-3,5,6-trichloro-2-pyridinecarboxyl are exemplary moieties in which R₃ is chloro and R₁ is hydrogen, chloro or methyl.

In some of any of the respective embodiments, R₃ is halo (optionally chloro) and R₁, R₂, R₄ and R₅ are each hydrogen. 4-Chlorophenoxyacetyl is an exemplary moiety in which R₃ is chloro and R₁, R₂, R₄ and R₅ are each hydrogen.

In some of any of the respective embodiments, R₃ is hydrogen or halo according to any of the respective embodiments described herein; and R₁ is hydrogen, halo or alkyl (e.g., C₁₋₄-alkyl) according to any of the respective embodiments described herein, and/or R₂ is hydrogen or amino (e.g., —NH₂) according to any of the respective embodiments described herein.

In some of any of the respective embodiments, R₄ is hydrogen or halo, optionally hydrogen or chloro. In some embodiments, R₄ is hydrogen.

In some of any of the respective embodiments, R₃ and R₄ are hydrogen or halo according to any of the respective embodiments described herein.

In some of any of the respective embodiments, R₄ is hydrogen or halo according to any of the respective embodiments described herein; and R₁ is hydrogen, halo or alkyl (e.g., C₁₋₄-alkyl) according to any of the respective embodiments described herein, and/or R₂ is hydrogen or amino (e.g., —NH₂) according to any of the respective embodiments described herein. In some such embodiments, R₃ is hydrogen or halo according to any of the respective embodiments described herein.

In some of any of the respective embodiments, R₅ is hydrogen or C₁₋₄-alkoxy, optionally hydrogen or methoxy. In some embodiments, R₅ is hydrogen.

In some of any of the respective embodiments, R₅ is hydrogen or C₁₋₄-alkoxy according to any of the respective embodiments described herein; and R₃ and/or R₄ are hydrogen or halo according to any of the respective embodiments described herein. In some such embodiments, R₁ is hydrogen, halo or alkyl (e.g., C₁₋₄-alkyl) according to any of the respective embodiments described herein. In some such embodiments, R₂ is hydrogen or amino (e.g., —NH₂) according to any of the respective embodiments described herein. In some such embodiments, R₁ is hydrogen, halo or alkyl (e.g., C₁₋₄-alkyl) according to any of the respective embodiments described herein, and R₂ is hydrogen or amino (e.g., —NH₂) according to any of the respective embodiments described herein.

In some of any of the respective embodiments, R₅ is hydrogen or C₁₋₄-alkoxy according to any of the respective embodiments described herein; and R₁ is hydrogen, halo or alkyl (e.g., C₁₋₄-alkyl) according to any of the respective embodiments described herein, and/or R₂ is hydrogen or amino (e.g., —NH₂) according to any of the respective embodiments described herein.

In some of any of the respective embodiments, R₁, R₃ and R₄ are each chloro. In some such embodiments, Y is N. 3,5,6-Trichloro-2-pyridinyloxyacetyl and 4-amino-3,5,6-trichloro-2-pyridinecarboxyl are exemplary moieties in which Y is N and R₁, R₃ and R₄ are each chloro.

In some of any of the respective embodiments, X is —O—CH₂— or —O—CH₂CH₂CH₂— (e.g., thus forming a phenoxyacetic acid or phenoxybutanoic acid, respectively). In some such embodiments, R₂ is hydrogen. In some embodiments, R₃ is chloro. In some embodiments, R₅ is hydrogen. In some embodiments, R₂ is hydrogen and R₃ is chloro. In some embodiments, R₂ and R₅ are each hydrogen. In some embodiments, R₅ is hydrogen and R₃ is chloro. In some embodiments, R₂ and R₅ are each hydrogen and R₃ is chloro.

In some of any of the respective embodiments, X is a bond. In some such embodiments, R₁ and R₄ are each chloro.

In some of any of the embodiments wherein X is a bond, Y is N and R₂ is amino (e.g., —NH₂). In some such embodiments, R₁, R₃ and R₄ are each chloro. 4-Amino-3,5,6-trichloro-2-pyridinecarboxyl (derived from the carboxylic acid known in the art as picloram) is an exemplary moiety in which X is a bond, Y is N, R₂ is amino, and R₁, R₃ and R₄ are each chloro.

In some of any of the embodiments wherein X is a bond, Y is CR₅, R₅ is methoxy, and R₃ is hydrogen. In some such embodiments, R₂ is hydrogen. In some embodiments, R₁ and R₄ are each chloro. In some such embodiments, R₂ is hydrogen and R₁ and R₄ are each chloro. 3,6-Dichloro-2-methoxybenzoyl (derived from the carboxylic acid known in the art as dicamba) is an exemplary moiety in which X is a bond, Y is CR₅, R₅ is methoxy, and R₂ and R₃ are each hydrogen, and R₁ and R₄ are each chloro.

In some of any of the respective embodiments, X is CH₂. In some such embodiments, Y is CR₅, and R₄ and R₅ together form a six-membered aromatic ring. R₁-R₃ are each optionally hydrogen. 1-naphthaleneacetyl is an exemplary acyl moiety wherein X is CH₂ and R₄ and R₅ together form a six-membered aromatic ring.

In some of any of the respective embodiments, R₇ is hydrogen or methyl. In some embodiments, R₇ is hydrogen, such that the compound is a conjugate of a primary amine (having the formula H₂NR₆, wherein R₆ is as defined in Formula I).

In some of any of the embodiments, R₆ and R₇ are such that the amino moiety is that of an amino acid, e.g., an L-amino acid or a D-amino acid, or an ester or amide thereof. The amino acid (optionally an L-amino acid) may be, for example, a natural amino acid such as alanine (Ala), arginine (Arg), asparagine (Asn), aspartate (Asp), cysteine (Cys), glutamine (Gln), glutamate (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and/or valine (Val), and/or an ester or amide thereof, e.g., wherein a carboxylic acid group thereof—or both carboxylic acid groups of Asp or Glu—is substituted (e.g., by alkyl) to form an ester or amide group (e.g., according to any of the respective embodiments described herein). In some such embodiments, the amino acid is other than glycine.

In some of any of the respective embodiments, the amino acid (e.g., L-amino acid) is a hydrophobic amino acid such as Ala, Val, Ile, Leu, Met, Phe, Tyr and/or Trp, optionally Val, Ile, Leu, Met, Phe and/or Trp (including esters and amides thereof).

In some of any of the respective embodiments, R₆ has Formula II:

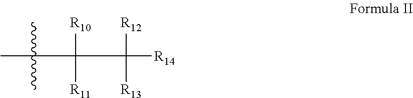

wherein:

R₁₀ and R₁₁ are each hydrogen, alkyl, alkenyl, alkynyl, cycloalkyl, aryl, heteroaryl, heteroalicyclic, carbonyl, thiocarbonyl, C-amido, and/or C-carboxy; and

R₁₂-R₁₄ are each individually hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and/or amino.

In some embodiments, R₆ has Formula II and R₇ is hydrogen or methyl. In some embodiments, R₆ has Formula II and R₇ is hydrogen.

In some of any of the embodiments relating to Formula II, R₁₀ is hydrogen or C-carboxy or C-amido. In some such embodiments, R₁₀ is C-carboxy or C-amido, optionally C-carboxy. In embodiments wherein R₁₀ is C-carboxy or C-amido, R₆ may be regarded as an alpha amino acid moiety (wherein R₁₀ is —C(═O)OH, optionally in a form of a salt, such as —C(═O)O⁻Na⁺) or ester thereof (e.g., wherein R₁₀ is —C(═O)OR₁₅, and R₁₅ is alkyl, alkenyl, alkynyl, cycloalkyl, heteroalicyclic, aryl or heteroaryl) or amide thereof. In some embodiments, R₁₅ is C₁₋₄-alkyl. In some exemplary embodiments, R₁₅ is methyl.

In some exemplary embodiments wherein R₁₀ is C-carboxy or C-amido, the C-carboxy is —C(═O)OCH₃ or —C(═O)OH and/or the C-amido is —C(═O)NH—(CH₂)₂-R₁₈, wherein R₁₈ is an ionic group, that is, a group which is ionic at a pH of 7. Examples of ionic groups include, without limitation, for example, —SO₃H, —PO₃H, and amino (e.g., quaternary ammonium groups such as trimethylamino).

In some of any of the embodiments relating to Formula II, R₁₁ is hydrogen. In some embodiments, R₁₁ is hydrogen and R₁₀ is hydrogen or C-carboxy or C-amido (according to any of the respective embodiments described herein), optionally C-carboxy or C-amido, and optionally C-carboxy.

In some of any of the embodiments relating to Formula II, neither R₁₀ nor R₁₁ is —C(═O)OH (or a deprotonated form or salt thereof). According to such embodiments, for example, when R₁₀ and R₁₁ are C-carboxy, the C-carboxy may be —C(═O)OR₁₅, and R₁₅ is alkyl, alkenyl, alkynyl, cycloalkyl, heteroalicyclic, aryl or heteroaryl.

In some of any of the embodiments relating to Formula II, R₁₂ is hydrogen, and R₁₃ is hydrogen or methyl. In some such embodiment, R₁₄ is hydrogen, —CH₃, —CH₂CH₃, —CH(CH₃)₂, —CH₂—S—CH₃, phenyl, 4-hydroxyphenyl, indol-3-yl, imidazol-4-yl, —CH₂CH₂NHC(═NH)NH, —CH₂CH₂CH₂NH₂, —C(═O)O—R₁₆, —CH₂C(═O)O—R₁₇, —C(═O)NH₂, —CH₂C(═O)NH₂, —OH and —SH, wherein R₁₆ and R₁₇ are each individually hydrogen or C₁₋₄-alkyl, optionally hydrogen or methyl. The skilled person will appreciate that such embodiments (e.g., wherein R₁₀ is C-carboxy and R₁₁ is hydrogen, according to any of the respective embodiments described herein) include moieties corresponding to almost all of the “standard” amino acids (including esters of glutamate and aspartate).

In some exemplary embodiments relating to Formula II, R₁₀ is —C(═O)OCH₃; R₁₁ and R₁₂ are each hydrogen; and R₁₃ and R₁₄ are each —CH₃ (corresponding to an L-valine methyl ester or D-valine methyl ester moiety), or R₁₃ is hydrogen and R₁₄ is indol-3-yl CH₃ (corresponding to an L-tryptophan methyl ester or D-tryptophan methyl ester moiety) or —C(═O)OCH₃ (corresponding to an L-aspartate methyl ester or D-aspartate methyl ester moiety).

Without being bound by any particular theory, it is believed that conjugates according to some embodiments described herein exhibit advantageous activity by being gradually hydrolyzed to release an active carboxylic acid, and that the structure of the amine modulates the rate of hydrolysis. For example, it is believed that amine moieties derived from amino acids with side chains (e.g., not glycine) or esters thereof are hydrolyzed more slowly than glycine-derived amine moieties, and that amine moieties derived from D-amino acids (or esters thereof) are hydrolyzed more slowly than corresponding amine moieties derived from L-amino acids (or esters thereof).

Similarly, it is believed that embodiments in which R₇ is hydrogen are generally hydrolyzed more rapidly (but not too rapidly) than embodiments in which R₇ is not hydrogen; and that embodiments in which R₇ is methyl are generally hydrolyzed more rapidly than embodiments in which R₇ is neither hydrogen nor methyl.

Thus, the rate of hydrolysis can be modulated, thereby modulating the nature of activity, as more gradual hydrolysis may be associated with lower toxicity but also lower potency.

In some of any of the embodiments relating to Formula I, R₇ is hydrogen or alkyl (e.g., methyl). In some such embodiments, R₇ is hydrogen and/or R₆ is not aryl.

In some embodiments, R₆ has Formula II and/or R₇ is hydrogen or alkyl (e.g., methyl), according to any of the respective embodiments described herein; and R₁ is hydrogen, halo or alkyl (e.g., C₁₋₄-alkyl) according to any of the respective embodiments described herein.

In some embodiments, R₆ has Formula II and/or R₇ is hydrogen or alkyl (e.g., methyl), according to any of the respective embodiments described herein; and R₂ is hydrogen or amino (e.g., —NH₂) according to any of the respective embodiments described herein. In some such embodiments, R₁ is hydrogen, halo or alkyl (e.g., C₁₋₄-alkyl) according to any of the respective embodiments described herein.

In some embodiments, R₆ has Formula II and/or R₇ is hydrogen or alkyl (e.g., methyl), according to any of the respective embodiments described herein; and R₃ and/or R₄ are hydrogen or halo according to any of the respective embodiments described herein. In some such embodiments, R₁ is hydrogen, halo or alkyl (e.g., C₁₋₄-alkyl) according to any of the respective embodiments described herein. In some such embodiments, R₂ is hydrogen or amino (e.g., —NH₂) according to any of the respective embodiments described herein. In some embodiments, R₁ is hydrogen, halo or alkyl (e.g., C₁₋₄-alkyl) according to any of the respective embodiments described herein, and R₂ is hydrogen or amino (e.g., —NH₂) according to any of the respective embodiments described herein.

In some embodiments, R₆ has Formula II and/or R₇ is hydrogen or alkyl (e.g., methyl), according to any of the respective embodiments described herein; and R₅ is hydrogen or C₁₋₄-alkoxy according to any of the respective embodiments described herein. In some such embodiments, R₃ and/or R₄ are hydrogen or halo according to any of the respective embodiments described herein. In some such embodiments, R₁ is hydrogen, halo or alkyl (e.g., C₁₋₄-alkyl) according to any of the respective embodiments described herein. In some such embodiments, R₂ is hydrogen or amino (e.g., —NH₂) according to any of the respective embodiments described herein. In some embodiments, R₁ is hydrogen, halo or alkyl (e.g., C₁₋₄-alkyl) according to any of the respective embodiments described herein, and R₂ is hydrogen or amino (e.g., —NH₂) according to any of the respective embodiments described herein. In some embodiments, R₁ is hydrogen, halo or alkyl (e.g., C₁₋₄-alkyl) according to any of the respective embodiments described herein; and R₃ and/or R₄ are hydrogen or halo according to any of the respective embodiments described herein. In some embodiments, R₂ is hydrogen or amino (e.g., —NH₂) according to any of the respective embodiments described herein; and R₃ and/or R₄ are hydrogen or halo according to any of the respective embodiments described herein.

In some embodiments, R₆ has Formula II and/or R₇ is hydrogen or alkyl (e.g., methyl), according to any of the respective embodiments described herein; R₅ is hydrogen or C₁₋₄-alkoxy according to any of the respective embodiments described herein; R₃ and/or R₄ are hydrogen or halo according to any of the respective embodiments described herein; R₂ is hydrogen or amino (e.g., —NH₂) according to any of the respective embodiments described herein; and R₁ is hydrogen, halo or alkyl (e.g., C₁₋₄-alkyl) according to any of the respective embodiments described herein.

In some of any of the embodiments relating to Formula I, R₆ is aryl, alkyl, alkenyl or alkynyl, wherein the alkyl is devoid of a —C(═O)OH substituent at the α-position thereof. In some such embodiments, R₇ is hydrogen or alkyl. In some embodiments, R₇ is hydrogen and/or R₆ is not aryl (i.e., R₆ is alkyl, alkenyl or alkynyl).

In some of any of the embodiments relating to Formula I, R₆ and R₇ together form a six-membered heteroalicyclic ring, for example, morpholine.

In some of any of the embodiments relating to Formula I, R₆ is aryl, alkyl, alkenyl and alkynyl, the alkyl being devoid of a —C(═O)OH substituent at the α-position thereof; R₇ is hydrogen or alkyl (e.g., methyl) according to any of the respective embodiments described herein, wherein when R₇ is alkyl, R₆ is not aryl, or alternatively, R₆ and R₇ together form a six-membered heteroalicyclic ring; and X, Y and R₁-R₅ are as defined herein according to any of the respective embodiments described herein. Compounds having Formula I meeting the aforementioned definitions are also referred to herein interchangeably as compounds having Formula Ia. Exemplary compounds according to Formula Ia are described in the Examples section herein.

In some of any of the embodiments relating to Formula Ia, R₆ has Formula II, according to any of the respective embodiments described herein, provided that neither R₁₀ nor R₁₁ is-C(═O)OH.

In some of any of the embodiments relating to Formula Ia, R₆ and R₇ are such that the amino moiety is that of an ester of an amino acid, e.g., an L-amino acid or a D-amino acid (e.g., according to any of the respective embodiments described herein), provided that the amino acid is not glycine. In some such embodiments, R₆ has formula II, wherein R₁₀ is —C(═O)O—R₁₅ (according to any of the respective embodiments described herein, wherein R₁₅ is not hydrogen) and R₇ is optionally hydrogen. In some exemplary embodiments, R₁₅ is methyl.

Methods and Uses:

The compounds of the present embodiments (e.g., compounds represented by Formula I as described herein in any of the respective embodiments) are usable, or are for use, in enhancing formation and/or growth of an adventitious root in a plant and/or plant tissue.

According to an aspect of embodiments of the invention, there is provided a method of enhancing formation and/or growth of an adventitious root in a plant and/or plant tissue. The method comprises contacting at least a portion of the plant and/or plant tissue with a compound having Formula I (according to any of the respective embodiments described herein).

Herein, an “adventitious root” refers to a root which originates from a stem, branch, leaf and/or woody portion of a plant, and which is not a primary root originating from a base of a plant. For example, an adventitious root may be a primary root which originates from any portion of a plant detached from the base of the plant (e.g., a cutting).

Herein “enhancing formation and/or growth” of a root encompasses increasing a probability that a root will form (e.g., increasing a percentage of cuttings in which root formation is effected), increasing a size (e.g., determined by length and/or volume) of the root(s) (e.g., after a given time, which may optionally reflect more rapid root growth), and/or increasing a number of roots (e.g., as determined by number of root termini) which form.

The nature of enhancing root formation and/or growth in a plant may optionally be determined by an obstacle to root formation and/or growth identified in said plant. For example, increasing a probability that a root will form (e.g., according to any of the respective embodiments described herein) may optionally be effected in a plant (e.g., cuttings thereof) identified as having a low probability (e.g., 20% or less of cuttings) of root formation (e.g., upon treatment with an auxin alone); increasing a root size (e.g., according to any of the respective embodiments described herein) may optionally be effected in a plant (e.g., cuttings thereof) identified as having a low root size (e.g., associated with slow root formation from cuttings), e.g. upon treatment with an auxin alone; and/or increasing a number of roots may optionally be effected in a plant (e.g., cuttings thereof) identified as having a low number of roots (e.g., when grown from cuttings), e.g. upon treatment with an auxin alone.

The skilled person will be capable of identifying particular obstacles to root formation and/or growth in particular plants, and accordingly determining suitable goals when applying a method described herein to such a plant, particularly in view of the abundant guidance presented herein.

The plant and/or plant tissue may optionally be in a form of a cutting, i.e., a portion of a plant (e.g., a portion comprising a stem and/or a leaf) separated from a plant.

The term “plant” as used herein encompasses whole plants, a grafted plant, ancestors and progeny of the plants and plant parts, including seeds, shoots, stems, roots (including tubers), rootstock, scion, and organs.

The term “plant tissue” encompasses, for example, roots, leaves, stems, flowers, seeds, fruits, plant cells (e.g., plant cell in an embryonic cell suspension, and/or a protoplast), suspension cultures, embryos, meristematic regions, callus tissue, leaves, gametophytes, sporophytes, pollen, and microspores, derived from any plant (as defined herein).

Plants that may be useful in the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia villosa, Pagopyrum spp., Feijoa sellowlana, Fragaria spp., Flemingia spp, Freycinetia banksli, Geranium thunbergii, Ginkgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a crop, a flower or a tree.

In some of any of the respective embodiments, the plant and/or plant tissue is of a type recognized as being difficult to root (e.g., exhibiting a resistance to adventitious root formation). A plant type may be characterized as difficult to root based on any of a variety of parameters, for example, species, maturity (e.g., wherein a mature plant tissue is less capable of root formation than a juvenile plant tissue), and/or region of a plant (e.g., from which a cutting is derived).

Optionally, the plant and/or plant tissue is of a species recognized in the art as being recalcitrant to adventitious root formation. Exemplary species include avocado, eucalyptus and pine trees.

Without being bound by any particular theory, it is believed that methods according to some embodiments are particularly advantageous in difficult-to-root plant samples; whereas in plant samples in which root formation is more readily obtained even in the absence of treatment, a treatment (e.g., according to a method described herein) provides less additional benefit.

In some of any of the respective embodiments, the plant is a woody plant, for example, a mature woody plant.

Herein, the term “woody plant” refers to a plant that produces wood as a structural tissue, and encompasses trees, shrubs and woody vines. The woody plant is optionally a gymnosperm or a dicot angiosperm.

Examples of woody plants include, without limitation, species of Actinidiaceae (e.g., Actinidia chinensis), Euphorbiaceae (e.g., Manihotesculenta), Lauraceae (e.g., avocado), Magnoliaceae (e.g., Firiodendron tulipifera), Myrtaceae (e.g., eucalyptus, for example, Eucalyptus botryoides, Eucalyptus camaldulensis, Eucalyptus dunnii, Eucalyptus globulus, Eucalyptus grandis, Eucalyptus kruseana, Eucalyptus loxophleba, Eucalyptus urophylla, and/or hybrids thereof such as Eucalyptus brachyphylla and/or Eucalyptus x trabutii), Salicaceae (e.g., Populus), Santalaceae (e.g., Santalum album), Ulmaceae (e.g., Ulmus), Rosaceae (e.g., Malus, Prunus, Pyrus), Rutaceae (e.g., Citrus, Microcitrus), and Gymnospermae (e.g., Picea spp. and Pinea spp.), forest trees (e.g., Betulaceae, Fagaceae, Gymnospermae and tropical tree species), fruit trees or shrubs, and oil palm.

Cuttings obtained from a woody plant may optionally be in a form of softwood cuttings (e.g., cuttings from stems that are rapidly expanding, with young leaves), semi-hardwood cuttings (e.g., from stems that have completed elongation growth and have mature leaves), and/or hardwood cuttings (e.g., fully matured stems, which are optionally dormant). It is to be appreciated that the terms “softwood cuttings” and “hardwood cuttings” refer to maturity of cuttings, and are not related to the classification of tree species into “softwood” and “hardwood” categories.

Techniques and conditions for growing cuttings are well known in the art.

Briefly, a high degree of moisture is typically desirable, as cuttings are susceptible to dehydration due to the initial lack of roots. The cuttings may optionally lack leaves (e.g., due to removal of at least a portion of the leaves, and/or taking the cutting from a dormant deciduous tree), which may limit water loss. Fungicides may be used to inhibit fungal growth, which may otherwise be encouraged by moist conditions. Soil which is particularly suitable for growth of cuttings may optionally be characterized by a pH of at least 6 (e.g., a pH of from 6 to 6.5), relatively high concentration of nutrients (e.g., obtainable by inclusion of humus or other organic substance), and/or sand or gravel (e.g., to enhance water permeability). Shade (optionally partial shade) and warmth (optionally warm soil in combination with cool air) may also be beneficial.

As an alternative to enhancing root formation and/or growth, or in addition to enhancing root formation and/or growth, compounds of the present embodiments (e.g., compounds represented by Formula I as described herein in any of the respective embodiments) are usable, or are for use, in promoting grafting unification, enhancing fruit size and/or in reducing flowering in a plant.

According to an aspect of embodiments of the invention, there is provided a method of promoting grafting unification in a plant. The method comprises contacting at least a portion of a plant (e.g., a scion and/or a rootstock which are to be grafted) with a compound having Formula I (according to any of the respective embodiments described herein).

Herein, the phrase “grafting” refers to a technique whereby tissues of a plant are joined in order that they continue to grow together, and the phrase “grafting unification” refers to successful grafting, that is, the joined tissues continue to grow together (e.g., the vascular tissues of the two parts grow together). Grafting typically involves forming a combined plant from an upper part of a plant (referred to as a “scion”), such as a cutting, grafted onto a plant or a portion of a plant comprising roots (referred to as a “rootstock”). Many suitable grafting techniques will be known to the skilled person.

Herein, the phrase “promoting grafting unification” encompasses increasing a percentage of grafts which undergo grafting unification and/or increasing a rate of growth of a grafted scion and/or the overall health of the combined plant following grafting.

A plant being grafted may be any plant described herein, and is optionally avocado.

According to an aspect of embodiments of the invention, there is provided a method of enhancing fruit size and/or reducing flowering in a plant. The method comprises contacting at least a portion of a plant (e.g., a fruit whose size is to be enhanced and/or a flower to be removed upon reduction) with a compound having Formula I (according to any of the respective embodiments described herein).

Herein, the phrase “reducing flowering” refers to reducing a number of flowers in a plant (also referred to as “diluting” flowers), optionally with the intention of reducing a number of fruits which develop thereafter.

Reduction of a number of fruits which develop may optionally be performed in order to enhance the size and/or quality of remaining fruits (e.g., wherein enhancing fruit size is effected at least in part by reducing flowering), to reduce a risk to a plant associated with excess fruits (e.g., a risk of buckling due to excess weight), to reduce fluctuations in fruit production (e.g., to reduce “alternate bearing”, a phenomenon in which a larger than average crop in one year tends to result in a smaller than average crop in the following year), and/or for economic reasons (e.g., to reduce harvest costs).

Examples of plants in which reducing flowering may optionally be effected include, without limitation, grape vine; stone fruit plants (e.g., trees), such as Prunus spp. (e.g., apricot, peach, nectarine, plum, cherry and/or almond) and mango; and pome fruit plants (e.g., trees), such as apple and pear.

In some of any of the respective embodiments, the method further comprises contacting at least a portion of the plant and/or plant tissue with an auxin.

Herein, the term “auxin” refers to a naturally occurring compound which acts as a hormone in plants (unless explicitly indicated otherwise).

Examples of suitable auxins include, without limitation, indole-3-acetic acid (a.k.a. indoleacetic acid or IAA), 4-chloroindole-3-acetic acid, phenylacetic acid, indole-3-butyric acid (a.k.a. indolebutyric acid or IBA) and indole-3-propionic acid. Indolebutyric acid (IBA) is an exemplary auxin.

Contacting the plant and/or plant tissue with an auxin may optionally be effected prior to, concomitantly with and/or subsequently to contacting the plant and/or plant tissue with a compound having Formula I. In exemplary embodiments, the plant and/or plant tissue is contacted with a composition comprising both the auxin and a compound having Formula I.

Contacting may be effected by any suitable technique, including, for example, dipping (e.g., dipping a base of a cutting in a composition comprising the active compound(s)) and/or spraying (e.g., spraying leaves of a cutting with a composition comprising the active compound(s)).

In some of any of the respective embodiments, the method comprises contacting at least one leaf of the plant (e.g., a cutting) with one or more compound having Formula I, optionally by spraying with a composition comprising the compound(s). In some such embodiments, the method further comprises contacting a base of a cutting with the compound(s) having Formula I, optionally by dipping the base in a composition comprising the compound(s). In exemplary embodiments, the method further comprises contacting a base of a cutting with an auxin (according to any of the respective embodiments described herein), e.g., IBA.

As exemplified herein, contacting the compound with both the base of a cutting and at least one leaf of a cutting may be particularly effective in enhancing rooting in cuttings.

In some of any of the embodiments relating to contacting with an auxin, a base of a cutting is contacted with the auxin, optionally by dipping the base in a composition comprising the auxin. Such a composition may optionally both the auxin and a compound having Formula I.

The compounds of some embodiments of the invention can be contacted with the plant and/or plant tissue per se, or in a composition (optionally a composition identified for use in enhancing formation and/or growth of an adventitious root in a plant and/or plant tissue), where it is mixed with a horticulturally acceptable carrier.

According to an aspect of embodiments of the invention, there is provided a composition for enhancing formation and/or growth of an adventitious root in a plant and/or plant tissue, the composition comprising a compound having Formula I (according to any of the respective embodiments described herein), as well as a horticulturally acceptable carrier (according to any of the respective embodiments described herein).

According to an aspect of embodiments of the invention, there is provided a composition for promoting grafting unification, enhancing fruit size and/or reducing flowering (e.g., reducing a number of flowers) in a plant, the composition comprising a compound having Formula I (according to any of the respective embodiments described herein), as well as a horticulturally acceptable carrier (according to any of the respective embodiments described herein).

The carrier, according to any of the respective embodiments of any of the aspects described herein, may optionally be in a form of a liquid, such as an aqueous carrier, and/or a particulate solid, such as talc.

Herein, the phrase “horticulturally acceptable carrier” refers to a carrier or a diluent that does not cause significant irritation or harm to a plant or plant tissue and does not abrogate the biological activity and properties of the administered compound.

The carrier may optionally comprise at least one excipient, that is, an inert substance added to a composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Additional ingredients which may optionally be comprised by a composition for enhancing root formation include, without limitation, fungicides suitable for horticultural use, such as diethofencarb, strobilurin fungicides (e.g., azoxystrobin, trifloxystrobin, kresoxim methyl, and strobilurin A, B, C, D, E, F, G and H), phenylamide fungicides (e.g., metalaxyl, mefenoxam), dicarboxymide fungicides (e.g., vinclozolin, iprodione, and procymidone), and benzimidazole fungicides (e.g., benomyl, carbendazim, thiophanate-methyl, thiabendazole, and fuberidazole).

The composition according to any of the respective embodiments described herein is optionally packaged in a packaging material and identified, in or on the packaging material for use in enhancing formation and/or growth of an adventitious root in a plant and/or plant tissue; optionally accompanied by instructions for use of the composition.

Compositions comprising a liquid carrier (according to any of the respective embodiments described herein) may optionally comprise one or more active compound (according to any of the respective embodiments described herein) dissolved in and/or suspended in the carrier, such as an aqueous carrier. Aqueous solutions may optionally be prepared by directly dissolving a water-soluble compound and/or by dissolving a compound in a water-soluble and/or water-miscible organic solvent, such as an alcohol (e.g., an ethanol), followed by dilution in an aqueous liquid.

Compositions comprising a solid carrier (according to any of the respective embodiments described herein), such as talc, may optionally comprise one or more active compound(s) (according to any of the respective embodiments described herein) adsorbed onto a surface of particles of the solid carrier, and/or in admixture with the solid carrier.

In some of any of the respective embodiments, a concentration of a compound having Formula I (according to any of the respective embodiments described herein) in a composition for being contacted with a plant or plant tissue is at least 10 nM. In some embodiments, the concentration is in a range of from 10 nM to 10 mM. In some embodiments, the concentration is in a range of from 10 nM to 1 mM. In some embodiments, the concentration is in a range of from 10 nM to 100 μM. In some embodiments, the concentration is in a range of from 10 nM to 10 μM. In some embodiments, the concentration is in a range of from 10 nM to 1 μM. In some embodiments, the concentration is in a range of from 10 nM to 100 nM.

In some of any of the respective embodiments, a concentration of a compound having Formula I (according to any of the respective embodiments described herein) in a composition for being contacted with a plant or plant tissue is at least 100 nM. In some embodiments, the concentration is in a range of from 100 nM to 10 mM. In some embodiments, the concentration is in a range of from 100 nM to 1 mM. In some embodiments, the concentration is in a range of from 100 nM to 100 μM. In some embodiments, the concentration is in a range of from 100 nM to 10 μM. In some embodiments, the concentration is in a range of from 100 nM to 1 μM.

In some of any of the respective embodiments, a concentration of a compound having Formula I (according to any of the respective embodiments described herein) in a composition for being contacted with a plant or plant tissue is at least 1 μM. In some embodiments, the concentration is in a range of from 1 μM to 10 mM. In some embodiments, the concentration is in a range of from 1 μM to 1 mM. In some embodiments, the concentration is in a range of from 1 μM to 100 μM. In some embodiments, the concentration is in a range of from 1 μM to 10 μM.

In some of any of the respective embodiments, a concentration of a compound having Formula I (according to any of the respective embodiments described herein) in a composition for being contacted with a plant or plant tissue is at least 10 μM. In some embodiments, the concentration is in a range of from 10 μM to 10 mM. In some embodiments, the concentration is in a range of from 10 μM to 1 mM. In some embodiments, the concentration is in a range of from 10 μM to 100 μM.

In some of any of the respective embodiments, a concentration of a compound having Formula I (according to any of the respective embodiments described herein) in a composition for being contacted with a plant or plant tissue is at least 100 μM. In some embodiments, the concentration is in a range of from 100 μM to 10 mM. In some embodiments, the concentration is in a range of from 100 μM to 1 mM.

In some of any of the respective embodiments, a concentration of a compound having Formula I (according to any of the respective embodiments described herein) in a composition for being contacted with a plant or plant tissue is at least 0.1 part per million (ppm) by weight. In some embodiments, the concentration is in a range of from 0.1 to 10,000 ppm by weight. In some embodiments, the concentration is in a range of from 0.1 to 1,000 ppm by weight. In some embodiments, the concentration is in a range of from 0.1 to 100 ppm by weight. In some embodiments, the concentration is in a range of from 0.1 to 10 ppm by weight. In some embodiments, the concentration is in a range of from 0.1 to 1 ppm by weight.

In some of any of the respective embodiments, a concentration of a compound having Formula I (according to any of the respective embodiments described herein) in a composition for being contacted with a plant or plant tissue is at least 1 part per million (ppm) by weight. In some embodiments, the concentration is in a range of from 1 to 10,000 ppm by weight. In some embodiments, the concentration is in a range of from 1 to 1,000 ppm by weight. In some embodiments, the concentration is in a range of from 1 to 100 ppm by weight. In some embodiments, the concentration is in a range of from 1 to 10 ppm by weight.

In some of any of the respective embodiments, a concentration of a compound having Formula I (according to any of the respective embodiments described herein) in a composition for being contacted with a plant or plant tissue is at least 10 parts per million (ppm) by weight. In some embodiments, the concentration is in a range of from 10 to 10,000 ppm by weight. In some embodiments, the concentration is in a range of from 10 to 1,000 ppm by weight. In some embodiments, the concentration is in a range of from 10 to 100 ppm by weight.

In some of any of the respective embodiments, a concentration of a compound having Formula I (according to any of the respective embodiments described herein) in a composition for being contacted with a plant or plant tissue is at least 100 parts per million (ppm) by weight. In some embodiments, the concentration is in a range of from 100 to 10,000 ppm by weight. In some embodiments, the concentration is in a range of from 100 to 1,000 ppm by weight.

Without being bound by any particular theory, it is believed that the more difficult to root a plant specimen is, the higher a concentration of active agent (e.g., a compound having Formula I and/or an auxin described herein) for rooting should be. Thus, for example, a concentration used for a woody plant (especially a mature woody plant) may be considerably higher than a concentration used for a non-woody plant.

In some of any of the respective embodiments, the composition further comprises an auxin (e.g., IBA) to be co-administered to the plant tissue, according to any of the respective embodiments described herein.

In some of any of the respective embodiments, a concentration of an auxin in a composition for being contacted with a plant or plant tissue (according to any of the respective embodiments described herein) is at least 10 nM. In some embodiments, the concentration of auxin is in a range of from 10 nM to 100 mM. In some embodiments, the concentration of auxin is in a range of from 10 nM to 10 mM. In some embodiments, the concentration of auxin is in a range of from 10 nM to 1 mM. In some embodiments, the concentration of auxin is in a range of from 10 nM to 100 μM. In some embodiments, the concentration of auxin is in a range of from 10 nM to 10 μM. In some embodiments, the concentration of auxin is in a range of from 10 nM to 1 μM. In some embodiments, the concentration of auxin is in a range of from 10 nM to 100 nM. In some embodiments, the auxin is IBA.

In some of any of the respective embodiments, a concentration of an auxin in a composition for being contacted with a plant or plant tissue (according to any of the respective embodiments described herein) is at least 100 nM. In some embodiments, the concentration of auxin is in a range of from 100 nM to 100 mM. In some embodiments, the concentration of auxin is in a range of from 100 nM to 10 mM. In some embodiments, the concentration of auxin is in a range of from 100 nM to 1 mM. In some embodiments, the concentration of auxin is in a range of from 100 nM to 100 μM. In some embodiments, the concentration of auxin is in a range of from 100 nM to 10 μM. In some embodiments, the concentration of auxin is in a range of from 100 nM to 1 μM. In some embodiments, the auxin is IBA.

In some of any of the respective embodiments, a concentration of an auxin in a composition for being contacted with a plant or plant tissue (according to any of the respective embodiments described herein) is at least 1 μM. In some embodiments, the concentration of auxin is in a range of from 1 μM to 100 mM. In some embodiments, the concentration of auxin is in a range of from 1 μM to 10 mM. In some embodiments, the concentration of auxin is in a range of from 1 μM to 1 mM. In some embodiments, the concentration of auxin is in a range of from 1 μM to 100 μM. In some embodiments, the concentration of auxin is in a range of from 1 μM to 10 μM. In some embodiments, the auxin is IBA.

In some of any of the respective embodiments, a concentration of an auxin in a composition for being contacted with a plant or plant tissue (according to any of the respective embodiments described herein) is at least 10 μM. In some embodiments, the concentration of auxin is in a range of from 10 μM to 100 mM. In some embodiments, the concentration of auxin is in a range of from 10 μM to 10 mM. In some embodiments, the concentration of auxin is in a range of from 10 μM to 1 mM. In some embodiments, the concentration of auxin is in a range of from 10 μM to 100 μM. In some embodiments, the auxin is IBA.

In some of any of the respective embodiments, a concentration of an auxin in a composition for being contacted with a plant or plant tissue (according to any of the respective embodiments described herein) is at least 100 μM. In some embodiments, the concentration of auxin is in a range of from 100 μM to 100 mM. In some embodiments, the concentration of auxin is in a range of from 100 μM to 10 mM. In some embodiments, the concentration of auxin is in a range of from 100 μM to 1 mM. In some embodiments, the auxin is IBA.

In some of any of the respective embodiments, a concentration of an auxin in a composition for being contacted with a plant or plant tissue (according to any of the respective embodiments described herein) is at least 1 mM. In some embodiments, the concentration of auxin is in a range of from 1 to 100 mM. In some embodiments, the concentration of auxin is in a range of from 1 to 10 mM. In some embodiments, the auxin is IBA.

In some of any of the respective embodiments, a concentration of an auxin in a composition for being contacted with a plant or plant tissue (according to any of the respective embodiments described herein) is at least 10 mM. In some embodiments, the concentration of auxin is in a range of from 10 to 100 mM. In some embodiments, the auxin is IBA.

In some of any of the respective embodiments, a concentration of an auxin in a composition for being contacted with a plant or plant tissue (according to any of the respective embodiments described herein) is at least 0.1 part per million (ppm) by weight. In some embodiments, the concentration of auxin is in a range of from 0.1 to 10,000 ppm by weight. In some embodiments, the concentration of auxin is in a range of from 0.1 to 1,000 ppm by weight. In some embodiments, the concentration of auxin is in a range of from 0.1 to 100 ppm by weight. In some embodiments, the concentration of auxin is in a range of from 0.1 to 10 ppm by weight. In some embodiments, the concentration of auxin is in a range of from 0.1 to 1 ppm by weight. In some embodiments, the auxin is IBA.

In some of any of the respective embodiments, a concentration of an auxin in a composition for being contacted with a plant or plant tissue (according to any of the respective embodiments described herein) is at least 1 part per million (ppm) by weight. In some embodiments, the concentration of auxin is in a range of from 1 to 10,000 ppm by weight. In some embodiments, the concentration of auxin is in a range of from 1 to 1,000 ppm by weight. In some embodiments, the concentration of auxin is in a range of from 1 to 100 ppm by weight. In some embodiments, the concentration of auxin is in a range of from 1 to 10 ppm by weight. In some embodiments, the auxin is IBA.

In some of any of the respective embodiments, a concentration of an auxin in a composition for being contacted with a plant or plant tissue (according to any of the respective embodiments described herein) is at least 10 parts per million (ppm) by weight. In some embodiments, the concentration of auxin is in a range of from 10 to 10,000 ppm by weight. In some embodiments, the concentration of auxin is in a range of from 10 to 1,000 ppm by weight. In some embodiments, the concentration of auxin is in a range of from 10 to 100 ppm by weight. In some embodiments, the auxin is IBA.

In some of any of the respective embodiments, a concentration of an auxin in a composition for being contacted with a plant or plant tissue (according to any of the respective embodiments described herein) is at least 100 parts per million (ppm) by weight. In some embodiments, the concentration of auxin is in a range of from 100 to 10,000 ppm by weight. In some embodiments, the concentration of auxin is in a range of from 100 to 1,000 ppm by weight. In some embodiments, the auxin is IBA.

In some of any of the respective embodiments, a concentration of an auxin in a composition for being contacted with a plant or plant tissue (according to any of the respective embodiments described herein) is at least 1,000 parts per million (ppm) by weight. In some embodiments, the concentration of auxin is in a range of from 1,000 to 10,000 ppm by weight. In some embodiments, the auxin is IBA.

### Additional Definitions

As used herein throughout, the term “alkyl” refers to any saturated aliphatic hydrocarbon including straight chain and branched chain groups. Preferably, the alkyl group has 1 to 20 carbon atoms.

Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted.

When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted.

Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted.

Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ or —N+R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is preferably not substituted by oxo, such that R′, R″ and R′″ are not (for example) carbonyl, C-carboxy or amide, as these groups are defined herein, unless indicated otherwise.

An “azide” group refers to a —N═N⁺═N⁻ group.

An “alkoxy” group refers to both an —O-alkyl and an —O-cycloalkyl group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to both an —S-alkyl group and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is defined as hereinabove.

A “thiocarbonyl” group refers to a —C(═S)—R′ group, where R′ is as defined herein.

A “carboxyl”, “carboxylic” or “carboxylate” refers to both “C-carboxy” and O-carboxy” groups, as defined herein.

A “C-carboxy” group refers to a —C(═O)—O—R′ group, where R′ is as defined herein. When R′ is H, the term “C-carboxy” refers to a carboxylic acid as defined herein.

An “O-carboxy” group refers to an R′C(═O)—O— group, where R′ is as defined herein.

A “carboxylic acid” refers to a —C(═O)OH group, including the deprotonated ionic form and salts thereof.

An “ester” refers to a —C(═O)OR′ group, wherein R′ is not hydrogen.

An “oxo” group refers to a═O group.

A “thiocarboxy” or “thiocarboxylate” group refers to both —C(═S)—O—R′ and —O—C(═S)R′ groups, where R′ is as defined herein.

A “halo” group refers to fluorine, chlorine, bromine or iodine.

A “haloalkyl” group refers to an alkyl group substituted by one or more halo groups, as defined herein.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″ group, where each of R′ and R″ is as defined herein.

A “carbamyl” or “carbamate” group encompasses O-carbamyl and N-carbamyl groups, as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

A “thiocarbamyl” or “thiocarbamate” group encompasses O-thiocarbamyl and N-thiocarbamyl groups, as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)-group, where Ra, Rb and Rd are as defined herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, agricultural and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

β-Alanine methyl ester (methyl 3-aminopropanoate) was obtained from Combi-Blocks, Inc.

2-Amino-5-methylpyridine was obtained from Sigma-Aldrich.

D-Aspartate methyl diester (D-Asp ME) was obtained from Combi-Blocks, Inc.

L-Aspartate methyl diester (L-Asp ME) was obtained from Combi-Blocks, Inc.

n-Butylamine was obtained from Sigma-Aldrich.

sec-Butylamine was obtained from Sigma-Aldrich.

1,1′-Carbonyldiimidazole (CDI) was obtained from Combi-Blocks, Inc.

4-CPA (4-chloro-phenoxyacetic acid) was obtained from Sigma-Aldrich.

Dichloromethane was obtained from Sigma-Aldrich.

Diethanolamine was obtained from Sigma-Aldrich.

2-DP (2-(2,4-dichlorophenoxy)propionic acid was obtained from Sigma-Aldrich.

Ethanolamine was obtained from Sigma-Aldrich.

Glycine methyl ester (Gly ME) was obtained from Combi-Blocks, Inc.

MCPA (2-methyl-4-chloro-phenoxyacetic acid) was obtained from Sigma-Aldrich.

Methanol was obtained from Sigma-Aldrich.

Methyl 4-aminobenzoate was obtained from Sigma-Aldrich.

Methyl 2-aminopyridine-4-carboxylate was obtained from Sigma-Aldrich.

N-Methylethanolamine was obtained from Sigma-Aldrich.

Morpholine was obtained from Sigma-Aldrich.

NAA (1-naphthaleneacetic acid) was obtained from Sigma-Aldrich.

3-Nitrotyrosine methyl ester was obtained from Sigma-Aldrich.

Piperidine was obtained from Sigma-Aldrich.

Tetrahydrofuran (THF) was obtained from Sigma-Aldrich.

o-Toluidine was obtained from Sigma-Aldrich.

p-Toluidine was obtained from Sigma-Aldrich.

Triethylamine was obtained from Sigma-Aldrich.

D-Tryptophan methyl ester (D-Val ME) was obtained from Combi-Blocks, Inc.

L-Tryptophan methyl ester (L-Val ME) was obtained from Combi-Blocks, Inc.

D-Valine methyl ester (D-Trp ME) was obtained from Combi-Blocks, Inc.

L-Valine methyl ester (L-Trp ME) was obtained from Combi-Blocks, Inc.

Rooting of Cuttings from Mature Eucalyptus grandis Trees:

Eight-year-old Eucalyptus grandis plants were grown from seeds and placed in a net house in 20 liter pots containing peat and tuff (70:30, v/v), drip irrigated and fertilized with 3 liters of Shefer™ 737 liquid fertilizer (ICL Fertilizers, Israel) per cubic meter of water. Cuttings were collected from branches which grew 2-2.5 meters above the ground. Cuttings were 2-3 mm thick branches, 15 cm long, with 1-2 pairs of leaves. The leaf blades were cut in half to decrease transpiration. Cuttings were treated with 6000 ppm IBA (potassium indole-3-butyric acid) for 1 minute with or without 100 μM of each tested compound. The tested compounds were either applied to the base of the cutting, with or without IBA and/or sprayed on the foliage in the presence of 0.05% Triton™ X-100 surfactant. Cuttings were rooted in rooting tables heated to 25° C. under constant 90% humidity, in a controlled-climate greenhouse. The rooting medium contained crushed polystyrene foam: vermiculite no. 3: pit (3:2:1, v/v/v). Rooting was recorded after 30-60 days. Roots system architecture was analyzed by WinRHIZO™ system scanner and software.

Induction of Lateral Root (LR) and Adventitious Root (AR) Formation in Arabidopsis Plants:

Adventitious roots (ARs) were induced in intact plants, using previously described procedures [Gutierrez et al., Plant Cell 2009, 21:3119-3132; Abu-Abied et al., Plant J 2012, 71:787-799; Rasmussen et al., Plant Physiol 2012, 158:1976-1987]. Briefly, seeds were germinated on MS/0.8% agar plates supplemented with 3% sucrose. The plates were kept in the dark for 2 days at 4° C., then 5 days at 22° C. in the dark, 2 days in the light, 3 days in the dark and then additional 4 days in the light to complete 2 weeks when roots were counted using a stereoscope. Sensitivity to auxin and/or auxin analogs was determined by following root elongation on vertical plates. The 4-day-old seedlings were transferred to MS plates containing 0.05 or 0.5 μM IAA and the root length was measured after 5 days, including the number of LRs in each root and calculation of the LR density. Each treatment experiment included 10-15 plants and was repeated 3 times.

Microscopy and Image Analysis:

Imaging was performed using an SP8 Leica confocal microscope including solid-state lasers producing 405, 488, 514 and 552 nm light, and hybrid or PMT detectors. Objectives were either PL APO 20×/0.75, WD 0.62 mm or PL APO 63×/1.2 WD 0.3 mm. For fluorescence measurements, the Imaris™ spot detection option (Bitplane A.G.) was used to segment nuclei and calculate the average signal intensity.

Liquid Chromatography-Mass Spectrometry (LC-MS) Analysis:

E. grandis cuttings were harvested at time 0, 1, 6 and 24 hours after the indicated treatments. The cutting foliage and basal ends (approximately 2-3 cm of the base) were harvested separately using a sharp pruning shear, rinsed well under a tap water stream, wiped with a paper towel and frozen in liquid nitrogen. Then, each sample was grinded well while still frozen, using an IKA lab mill. For the analysis, 3-6 technical replicates, weight 190-240 mg, were taken from each sample into fresh 2 ml Eppendorf™ tubes. Auxins were extracted in 1 ml of cold 79% isopropanol, 20% methanol and 1% acetic acid solution containing 20 ng of 12C labeled IBA and IAA as internal standards. The tubes were vortexed for 1 hr at 4° C. and then centrifuged at 14,000 RPM for 15 minutes. The supernatants were transferred to fresh 2 ml Eppendorf™ tubes. Two more extraction cycles were performed using 0.5 ml of extraction solvent without the internal standards. The tubes containing the collected supernatants were placed in a SpeedVac™ centrifuge for solvent evaporation under room temperature. The pellets were dissolved in pre-chilled 200 μl of 50% methanol, centrifuged, and the supernatant was filtered through 0.22 m PDFV syringe filters 13 mm into fresh 2 ml tubes. The ready extractions were kept under −20° C. till analyzed.

LC-MS analyses were conducted using UPLC-Triple Quadrupole-MS device (Waters Xevo TQ MS). Separation was performed on Waters Acquity™ UPLC BEH C18 1.7 μm 2.1×100 mm column with a VanGuard™ precolumn (BEH C18 1.7 μm 2.1×5 mm). Chromatographic and MS parameters were as follows: the mobile phase consisted of water (phase A) and acetonitrile (phase B), both containing 0.1% formic acid in the gradient elution mode. The solvent gradient program was as follows:

| Time (minutes) | Phase A % | Phase B % |
|---|---|---|
| Initial | 95 | 5 |
| 0.5 | 95 | 5 |
| 7.0 | 40 | 60 |
| 8.0 | 5 | 95 |
| 11 | 5 | 95 |
| 12 | 95 | 5 |
| 15 | 95 | 5 |

The flow rate was 0.3 ml/minute, and the column temperature was kept at 35° C. The analyses were performed using the ESI source in negative ion mode with the following settings: capillary voltage 3.1 kV, cone voltage 30 V, desolvation temperature 400° C., desolvation gas flow 565 liters/hour, source temperature 140° C. Quantitation was performed using MRM acquisition by monitoring the 185/127, 185/141, RT=5.77, dwell time of 161 msec for each transition. Calibration curve was used to calculate the concentration of 4-CPA. Acquisition of LC-MS data was performed under MassLynx™ V4.1 software (Waters).

RNA Preparation:

E. grandis cuttings were treated with either 6000 ppm IBA by submerging the cutting base for 1 minute or by submerging the cutting base in 6000 ppm IBA with 100 μM 4-CPA-L-Trp, for 1 minute and spraying the leaves with 100 μM 4-CPA-L-Trp. Controls were untreated cuttings. For each treatment, 20 cuttings were used all from the same clonal tree. Twenty four hours after the treatments, the cuttings were taken out of the rooting table, rinsed well and then cambium cells and immature xylem cells were isolated from them according to procedures such as described by Foucart [New Phytol 2006, 170:739-752] and Ridoutt et al. [Plant Cell Physiol 1995, 36:1143-1147]. Briefly, bark from the 2 cm in the basal end of each cutting was peeled off using a sharp scalpel. Then, the tissue from the inner side of the bark and from the outer side of the remaining stem was scraped gently and immediately frozen in an Eppendorf™ tube in liquid nitrogen. An average yield of tissue from 20 cuttings was found to be 50 mg. RNA from this tissue was extracted using a Norgen Biotek RNA Extraction kit (cat. #25800) according to the basic manufacturer protocol, including an on-column DNAase treatment. Samples were sent for sequencing to Macrogen laboratories in South Korea.

Bioinformatics Analysis:

RNA sequencing raw-reads were subjected to a filtering and cleaning procedure. The FASTX Toolkit (www(dot)hannonlab(dot)cshl(dot)edu/fastx_toolkit/index(dot)html, version 0.0.13.2) was used to trim read-end nucleotides with quality scores <30, using the FASTQ Quality Trimmer, and to remove reads with less than 70% base pairs with a quality score ≤30 using the FASTQ Quality Filter. Clean-reads were aligned to the Eucalyptus grandis genome extracted from Phytozome database (Eucalyptus_grandisv2; www(dot)phytozome(dot)jgi(dot)doe(dot)gov/pz/portal(dot)html) using Tophat2 software (v2.1) [Kim et al., Genome Biol 2013, 14:R36]; gene abundance estimation was performed using Cufflinks (v2.2) [Trapnell et al., Nat Biotechnol 2010, 28:511-515], combined with gene annotations from the Phytozome. Differential expression analysis was completed using the DESeq2 R package. Genes that varied from the control more than twofold, with an adjusted P-value of no more than 0.05, were considered differentially expressed. Venn diagrams were calculated using “Venny” tool [Oliveros, J. C. (2007-2015) Venny. An interactive tool for comparing lists with Venn's diagrams. www(dot)bioinfogp(dot)cnb(dot)csic(dot)es/tools/venny/index(dot)html)/] (or www(dot)bioinformatics(dot)psb(dot)ugent(dot)be/webtools/Venn/ web tool) based on the Arabidopsis database (TATR; www(dot)Arabidopsis(dot)org/) homology accessions.

### Example 1

### Preparation of Conjugates of Auxin Analogs

Four synthetic auxin analogs were chosen as active compounds: 4-CPA (4-chloro-phenoxyacetic acid), MCPA (2-methyl-4-chloro-phenoxyacetic acid), 2-DP (2-(2,4-dichlorophenoxy)propionic acid and NAA (1-naphthaleneacetic acid). Each of the 4 auxin analogs was conjugated to various amines by an amide bond or to an alcohol (methanol) by an ester bond.

Conjugates of the phenoxy acids (4-CPA, MCPA and 2-DP) were synthesized as a one-pot procedure, such as depicted in Scheme 1, in which the carboxylic group of the phenoxy acids was first activated by the coupling reagent 1,1′-carbonyldiimidazole (CDI) and subsequently reacted with the appropriate amide. The obtained conjugates were typically in a range of from 65-90%.

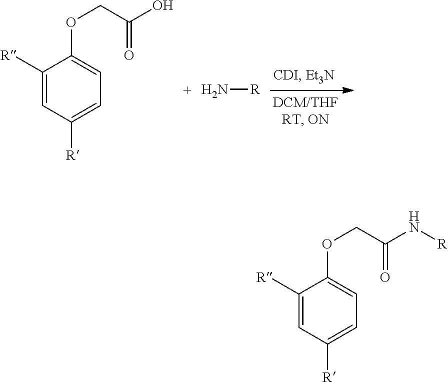

NAA was not sufficiently reactive under the abovementioned conditions, and was therefore converted to the corresponding acyl chloride, using oxalyl chloride, prior to reaction with amines.

Using the above general procedures, each of the abovementioned four auxin analogs was conjugated to 7 different amines, ethanolamine, β-alanine methyl ester (methyl 3-aminopropanoate), methyl 4-aminobenzoate, p-toluidine, o-toluidine, methyl 2-aminopyridine-4-carboxylate, and 2-amino-5-methylpyridine.

In an exemplary synthesis, a solution of 4-CPA in 30 ml dichloromethane (DCM) and a few drops of tetrahydrofuran (THF) was prepared and 1.05 molar equivalents of CDI and 2.1 molar equivalents of triethylamine (Et₃N) were added. After stirring the solution for 2 hours at room temperature, 1.05 molar equivalents of an amine was added. The reaction was monitored by thin-layer chromatography (TLC) to determine its completion (typically 1-2 hours). After completion, the reaction mixture was washed with 1 M HCl, brine, and then water, and the organic phase was separated, dried over MgSO₄ and concentrated under vacuum. If needed, the crude residue was purified by silica gel chromatography (ethyl acetate:hexane). The yield was in a range of from 45% to 90%.

Based on the results presented in Examples 2 and 3, additional conjugates were prepared according to procedures described hereinabove between 4-CPA and 6 amines selected as structural analogs of ethanolamine, but expected to be more resistant to hydrolysis upon conjugation; as well as between nitrotyrosine methyl ester and each of 4-CPA, MCPA and 2-DP. The 6 amines selected as analogous to ethanolamine were sec-butylamine, n-butylamine, piperidine, morpholine, diethanolamine, and N-methylethanolamine.

Based on the results obtained with the abovementioned conjugates (e.g., as presented in Example 3), additional conjugates were prepared according to procedures described hereinabove between 4-CPA and methyl esters of D- and L-isomers of the amino acids valine, aspartic acid (methyl diester), and tryptophan (for brevity, the methyl esters of amino acids conjugated to 4-CPA are also referred to herein simply by the name of the amino acid).

The conjugates prepared as described hereinabove are summarized in FIG. 1.

### Example 2

### Effect of Conjugates on Root Formation in Mung Bean Cuttings Model

Mung bean cuttings have been used for many years as a model for assessing the effect of plant hormones and synthetic chemicals on the rooting process. In the present study, this system was used to ascertain activity of conjugates in inducing adventitious root (AR) formation, and to evaluate the rate of conjugate hydrolysis that releases the free active auxin. As discussed herein, slow hydrolysis may neutralize or decrease the phytotoxicity of highly active auxins and ensure a desirable prolonged supply of auxin. The rooting activity in this system is determined by the number of adventitious roots per cutting.

FIGS. 2A-2C show examples of enhancement of rooting in mung bean cuttings by IBA (FIGS. 2B and 2C) and 2-DP-Gly-ME (FIGS. 2A and 2C).

The mung bean model was used to examine the activity of conjugates of three phenoxy acids, 4-CPA, MCPA, and 2-DP, on rooting. For comparison, plants were treated with (unconjugated) IBA or with 2-DP conjugated to glycine methyl ester (Gly-ME).

As shown in Table 1 below, the tested 2-DP conjugates generally induced AR formation at concentrations of 10 and 50 μM, the effect of tested MCPA conjugates was highly variable, and the tested 4-CPA conjugates generally failed to induce AR formation at the examined concentrations, with the exception of the conjugate with ethanolamine, which gave positive results at both 10 and 50 μM.

These results suggest that the root formation effect of ethanolamine conjugates is associated with a relatively slow hydrolysis rate, and that the inhibitory activity of other 4-CPA conjugates is due to toxicity associated with rapid hydrolysis of these conjugates. This conclusion is consistent with data in the literature suggesting that the auxin activity of 2-DP is weaker than that of 4-CPA and MCPA.

TABLE 1
Effect of conjugates of phenoxy acids with various amines on rooting of mung bean
cuttings (values higher than control treatment with water (29.6) are in bold); IBA and
2-DP conjugate with glycine methyl ester were used for comparison.

| Compound | | | Concentration (μM) | |
|---|---|---|---|---|
| No. | Auxin | Conjugated molecule | 10 | 50 |
| | IBA | none | | 81.0 |
| | 2-DP | glycin methyl ester | | 97.2 |
| 1a | 4-CPA | ethanolamine | 49.5 | 14.9 |
| 2a | MCPA | | 34.7 | 72.1 |
| 3a | 2-DP | | 26.4 | 24.1 |
| 1b | 4-CPA | β-alanine methyl ester | 0 | 0 |
| 2b | MCPA | | 94.8 | 63.2 |
| 1c | 4-CPA | 2-amino-5-methylpyridine | | 0 |
| 2c | MCPA | | 0 | 0 |
| 1d | 4-CPA | methyl 2-aminopyridine-4-carboxylate | 0 | 0 |
| 2d | MCPA | | 64.3 | 0 |
| 3d | 2-DP | | 41.6 | 85.8 |
| 1e | 4-CPA | o-toluidine | | 0 |
| 2e | MCPA | | 23.6 | 0 |
| 3e | 2-DP | | 37.3 | 41.7 |
| 1f | 4-CPA | p-toluidine | 0 | 0 |
| 2f | MCPA | | 0 | 0 |
| 1g | 4-CPA | methyl 4-aminobenzoate | 0 | 0 |
| 2g | MCPA | | 0 | 0 |
| 3g | 2-DP | | 46.2 | 46.4 |

In order to confirm the above conclusions, the activity of the conjugates of 2-DP and 4-CPA with glycine (methyl ester) (Gly-ME) was compared to that of free 2-DP and 4-CPA.

As shown in FIG. 3, both free 2-DP and 2-DP conjugated to Gly-ME exhibited an effect on AR formation which was positively correlated to dose.

In contrast, as shown in FIGS. 4A-4C, both free 4-CPA and 4-CPA conjugated to Gly-ME induced a similar rooting rate only at the lowest tested concentration of 2 μM, which was almost as high as the rate obtained with 50 μM IBA.

As further shown in FIGS. 4A and 4B, the base of cuttings treated with free (i.e., unconjugated) 4-CPA (FIG. 4B) or with 4-CPA conjugated to Gly-ME (FIG. 4A) appeared swollen, but no AR developed.

Similar results were obtained with other compounds which inhibited root development (not shown).

These results indicate the occurrence of cell division, albeit with inhibition of the differentiation of adventitious roots or their elongation.

Taken together, the above results indicate that inhibition of root formation in this model is associated with strong auxin activity, and such strong activity is affected by the potency of the free auxin analog (e.g., 4-CPA is more active than 2-DP) and by the rate of release of the free auxin analog by hydrolysis, with faster hydrolysis resulting in stronger inhibition.

These results further indicate that 4-CPA has a relatively high rooting activity (provided levels of 4-CPA are low enough to avoid toxicity), and suggest that conjugates which are not rapidly hydrolyzed would be more effective by avoiding a high phytotoxic level of 4-CPA.

In view of the above, additional conjugates that might exhibit slow hydrolysis were prepared. These conjugates were synthesized from various amines selected as bulkier analogs of ethanolamine, including various D- and L-amino acids (in the form of methyl esters). It was hypothesized that D-amino acids, which are not the common amino acid form present in plant tissues, would be hydrolyzed at a relatively slow rate.

As shown in Table 2 below, N-methylethanolamine conjugate of 4-CPA exhibited a moderate rooting activity at 50 μM (the highest concentration examined), whereas piperidine, morpholine, n-butylamine and sec-butylamine conjugates did not. These results indicate that the N-methylethanolamine conjugate underwent a relatively slow hydrolysis, whereas the other conjugates underwent faster hydrolysis which resulted in phytotoxicity.

TABLE 2
Effect of conjugates of 4-CPA with various amines on rooting of mung
bean cuttings (IBA used for comparison).

| Compound | | | Concentration (μM) | | |
|---|---|---|---|---|---|
| No. | Auxin | Conjugated molecule | 2 | 10 | 50 |
| | IBA | | | | 101 |
| 1i | 4-CPA | sec-butylamine | 23 | 25 | 0 |
| 1j | 4-CPA | n-butylamine | 22 | 0 | 0 |
| 1k | 4-CPA | piperidine | 25 | 34 | 0 |
| 1l | 4-CPA | morpholine | 23 | 0 | 0 |
| 1n | 4-CPA | N-methylethanolamine | 16 | 24 | 35 |
| 1q | 4-CPA | D-Asp-methyl ester | 67 | 0 | 0 |
| 1r | 4-CPA | L-Asp-methyl ester | 121 | 34 | 0 |

As shown in FIGS. 5A and 5B, the D-Val-methyl ester and D-Trp-methyl ester conjugates (Compounds 1o and 1s, respectively) induced rooting to a degree positively correlated with concentration, with a high rooting rate at the highest concentration examined, whereas the L-Val-methyl ester and L-Trp-methyl ester conjugates (Compounds 1p and 1t, respectively) induced rooting only at low concentrations. These results indicate that the D-amino acid conjugates underwent a relatively slow hydrolysis, whereas the L-amino acid conjugates underwent excessively fast hydrolysis which resulted in phytotoxicity.

As further shown in Table 2, the Asp-methyl ester conjugates surprisingly behaved differently from the other amino acid conjugates, as the L-Asp-methyl ester conjugate (Compound 1r) exhibited more activity than the D-Asp-methyl ester conjugate (Compound 1q) at low and intermediate concentrations (2 and 10 μM), suggesting that the L-Asp-methyl ester conjugate is hydrolyzed more slowly than the D-Asp-methyl ester conjugate.

Taken together, the above results indicate that 4-CPA conjugates with a low hydrolysis rate can be prepared using specific amines and amino acids such as ethanolamine, N-methylethanolamine, D-Val-methyl ester, and D-Trp-methyl ester, and that such conjugates are effective at promoting root formation.

### Example 3

### Effect of Conjugates on Root Formation in Eucalyptus grandis Cutting Model

Exemplary conjugates prepared as described in Example 1 were tested for their ability to promote adventitious root (AR) formation in cuttings from mature eucalyptus (Eucalyptus grandis) trees. In this model, AR formation is difficult to induce, with 5-15% root development in the presence of IBA potassium salt [Abu-Abied et al., Plant J 2012, 71:787-799], such that effective AR formation indicates a potent root formation activity.

Various concentrations and mode of applications were tested for each compound in the presence or absence of IBA (the “gold standard” rooting enhancer). A concentration of 100 μM of each conjugate was used for an initial screen of 37 conjugates, in which the compounds were applied to the base of the cutting by dipping (submerging the cutting base in a solution of the conjugate for one minute) or to the foliage by spraying. After 45 days, the cuttings were scored for the presence of callus or roots.

As shown in FIG. 6, Compounds 1a (4-CPA-ethanolamine), 3h (2-DP-methanol), 1b (4-CPA-β-alanine methyl ester), 1g (4-CPA-methyl-4-aminobenzoate), 1f (4-CPA-p-toluidine) and 3f (2-DP-p-toluidine) in combination with IBA enhanced rooting in comparison with IBA alone, under at least some of the tested conditions.

As further shown in FIG. 6, 100 μM Compound 1a (4-CPA-ethanolamine) also promoted rooting in the absence of IBA, at an efficiency at least as high as that of 28 mM IBA (15% versus 12%, respectively), despite being applied at a 280-fold lower concentration.

These results indicate that the phytoxic effect of the auxin analogs is reduced considerably by conjugation.

As further shown in FIG. 6, either submerging the cutting base or spraying on leaves could result in enhanced rooting. This result indicates that the rooting promotion is a systemic effect, and is not associated primarily with local application of the compounds near the roots.

As most of the abovementioned compounds which enhanced the efficacy of IBA are 4-CPA conjugates, further investigations were performed on various conjugates of 4-CPA, with particular attention to 4-CPA-ethanolamine conjugate and analogs thereof (as described in Example 1 hereinabove, e.g., in Round #2 of FIG. 1).

As shown in FIG. 7, the combination of IBA with conjugates reduced callus formation as compared to conjugate alone (up to 22% versus up to 72%, respectively).

Although callus formation is often a necessary preliminary to rooting, roots only seldom arise directly from the callus [Abu-Abied et al., Plant J 2012, 71:787-799], and in other cases, the callus forms a dead end in the process of rooting [Hartmann et al., Hartmann and Kester's Plant Propagation Principles and Practices, Eighth Edition, Pearson Education Limited, Essex, Great Britain (2011)].

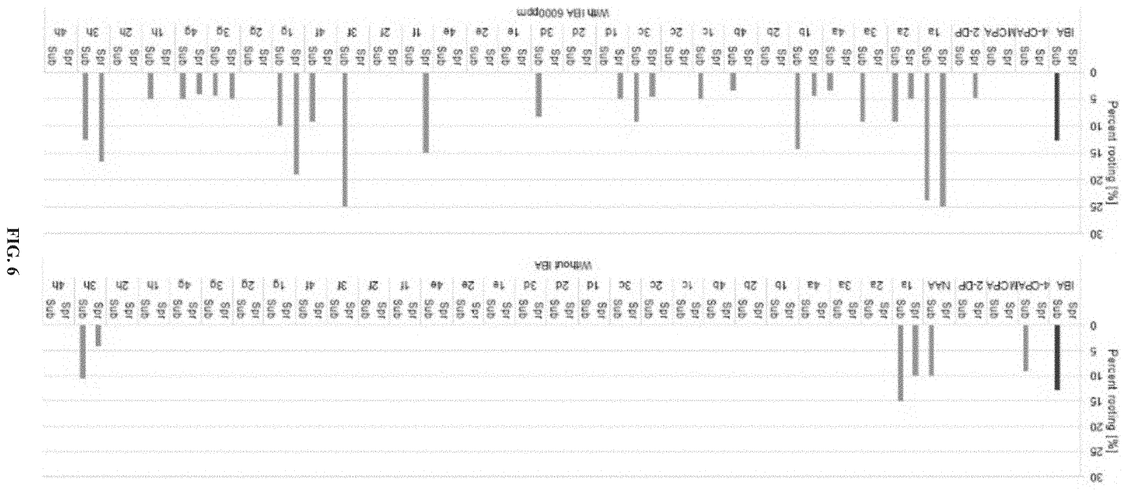

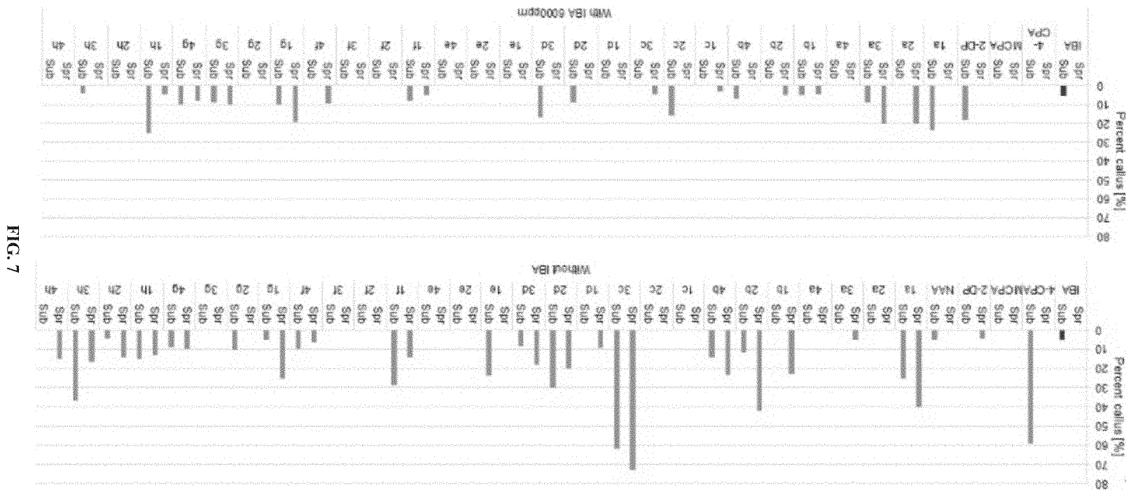

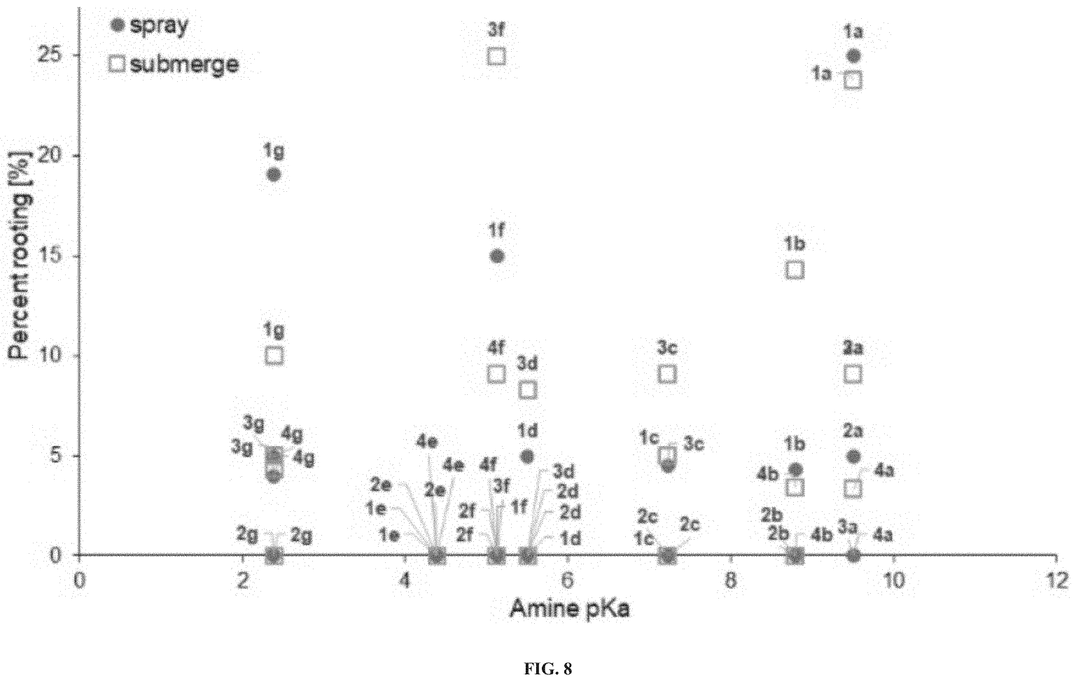

As shown in FIG. 8, conjugates formed from an amine with a lower pKa (e.g., a pKa lower than 8.0 or 9.0) tended to be more likely to exhibit little or no rooting enhancement.

Without being bound by any particular theory, it is believed that lower amine pKA is associated with more labile amide bonds, and that the reduction in activity at lower pKA values is associated with relatively rapid hydrolysis of the conjugate, which can lead to phytotoxicity. It is further believed that an amine with a relatively high pKa (e.g., a pKa above 10.5 or 11.0) may undergo hydrolysis too slowly to exhibit maximal activity.

In view of the above, further conjugates were mostly prepared from amines (e.g., primary alkylamines) having a pKa in a range of 8.3 to 11.2.

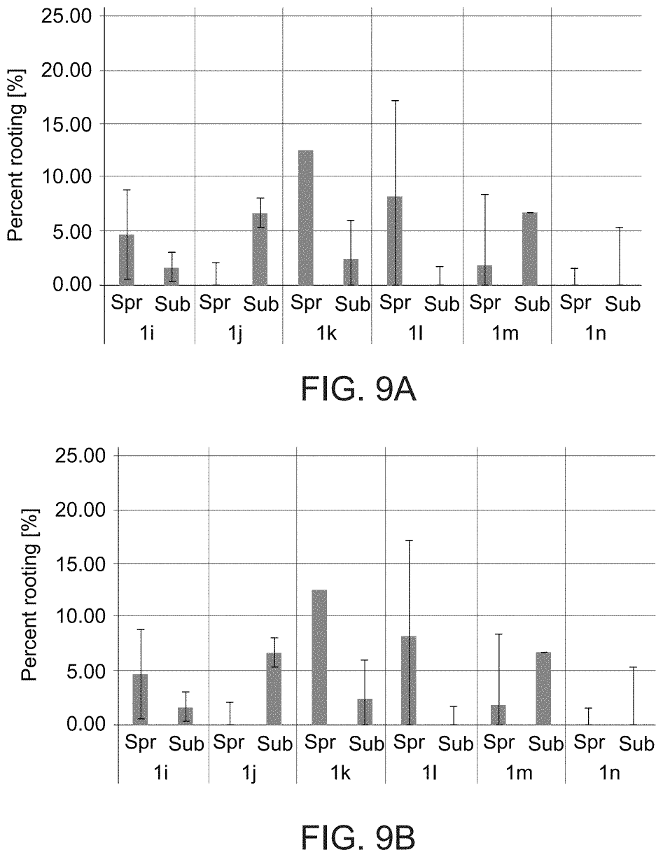

As shown in FIGS. 9A and 9B, conjugates of 4-CPA with sec-butylamine (Compound 1i), n-butylamine (Compound 1j), piperidine (Compound 1k), morpholine (Compound 1l), diethanolamine (Compound 1m) and N-methylethanolamine (Compound 1n) were not more effective at inducing rooting than was the conjugate of 4-CPA with ethanolamine (Compound 1a), and in some cases were less effective.

The above results suggest that slower hydrolysis than that of the ethanolamine conjugate is not advantageous in this model.

In view of the relatively positive results obtained (as described hereinabove) with conjugates of ethanolamine (a primary alkylamine), further investigations were performed with conjugates of 4-CPA and primary alkylamines such as amino acids. It was hypothesized that amino acids would serve as highly biocompatible primary alkylamines (e.g., upon hydrolysis of the conjugate) and result in reasonably water-soluble conjugates.

It was further hypothesized that hydrolysis may be controlled by enzymes which differentiate between biologically atypical D-amino acids and typical L-amino acids, thereby facilitating control over the hydrolysis rate.

4-CPA conjugates were therefore prepared with the methyl esters of D-valine (Compound 1o) and L-valine (Compound 1p) (an example of a hydrophobic amino acid), D-aspartate (Compound 1q) and L-aspartate (Compound 1r) (an example of a hydrophilic amino acid), and D-tryptophan (Compound 1s) and L-tryptophan (Compound 1t) (an example of an aromatic amino acid), as described in Example 1 hereinabove, e.g., in Round #3 of FIG. 1. The conjugate of 4-CPA and glycine methyl ester was used as a control.

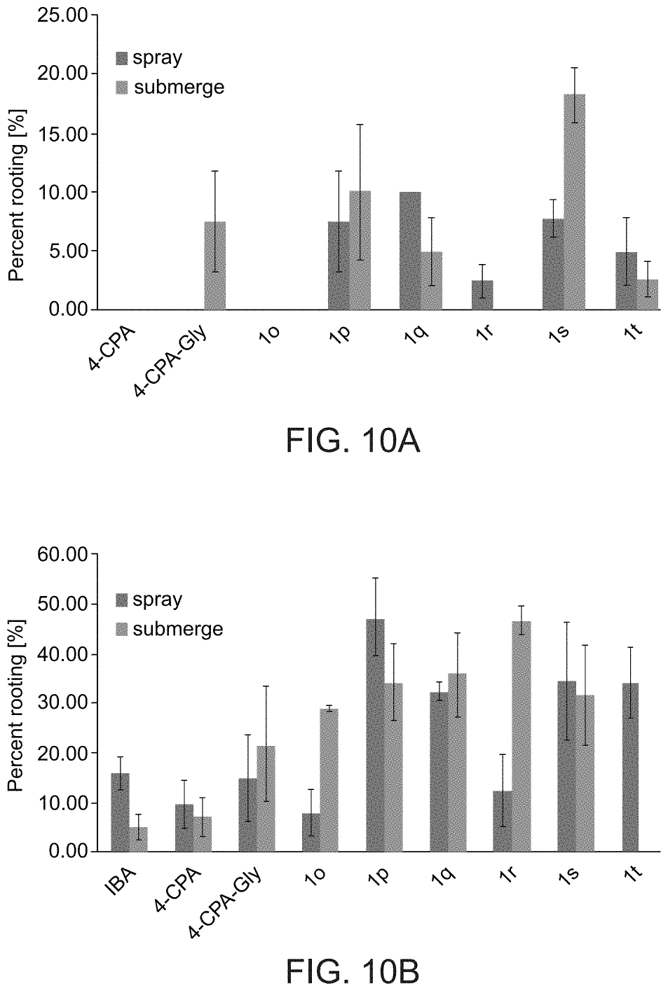

As shown in FIGS. 10A and 10B, each of the tested amino acid conjugates could repeatedly promote rooting, with up to 18% rooting as a stand-alone treatment (FIG. 10A), and up to 47% rooting when applied with IBA; whereas the 4-CPA-Gly conjugate exhibited lower rooting activity than the other conjugates, and treatments with IBA and/or free 4-CPA were considerably less effective than those with IBA and 4-CPA conjugates.

As further shown in FIGS. 10A and 10B, L-amino acid conjugates (Compounds 1p, 1r and 1t) exhibited activity which was at least as potent as that of their corresponding D-amino acid conjugates (Compounds 1o, 1q and 1s, respectively), which are presumably more resistant to hydrolysis.

Taken together, the above results indicate that greater resistance to hydrolysis than that exhibited by the exemplary ethanolamine or L-amino acid conjugates is not advantageous for promoting effects such as Eucalyptus grandis AR formation, where potent activity is necessary.

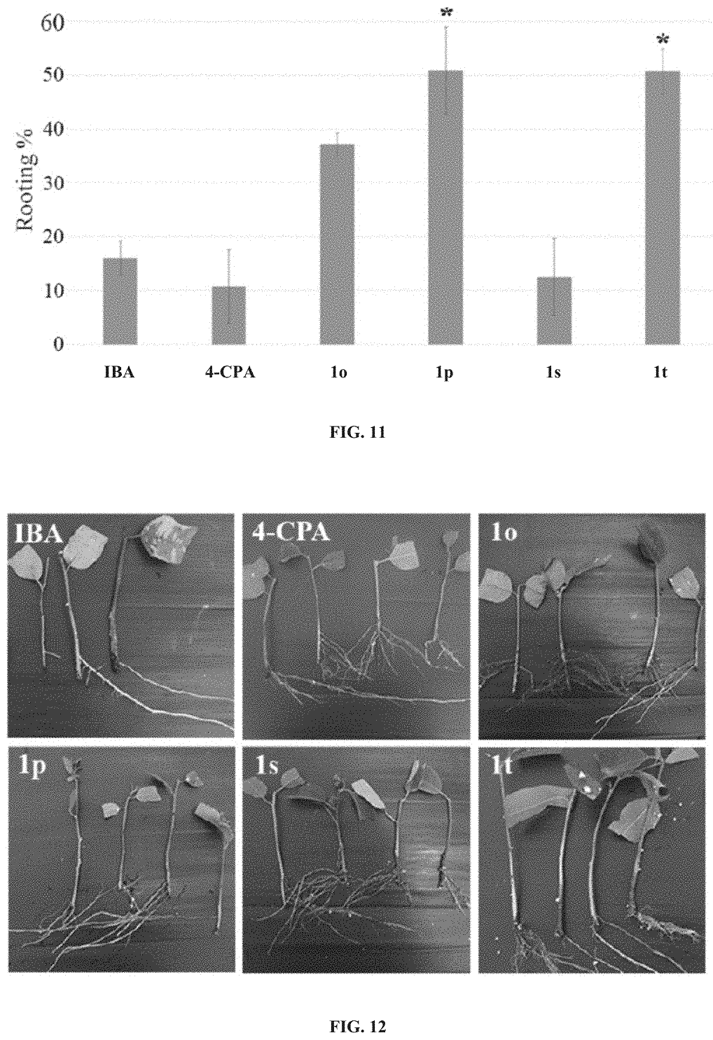

As shown in FIG. 11, submerging the cutting base in IBA and conjugate in addition to spraying with the conjugate consistently provided a particularly strong rooting effect, with 51±8% rooting for Compound 1p and 51±7% rooting for Compound 1t (in both cases, p<0.05 relative to IBA alone).

As shown in FIG. 12, the roots which developed upon treatment with Compounds 1o, 1p, 1s or 1t appeared more branched than those which developed upon treatment with IBA alone.

Similar results were obtained with Compounds 1q and 1r (data not shown).

In order to quantify the differences in root system architectures, the roots were analyzed by a WinRHIZO™ image analysis system.

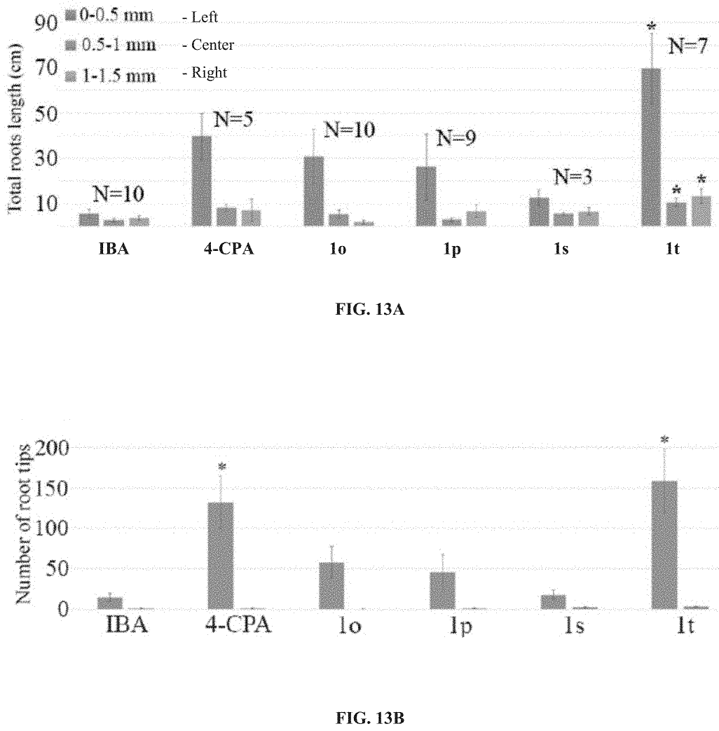

As shown in FIG. 13A, application of IBA with exemplary compounds (especially Compound 1t) considerably enhanced the total root length in comparison with IBA alone, especially with respect to total length of thin roots.

As shown in FIG. 13B, application of IBA with exemplary compounds (especially Compound 1t) considerably enhanced the number of root tips in comparison with IBA alone.

These results indicate that the conjugates enhance root branching and formation of thin lateral roots, including in cases where the average number of main roots is not increased.

Taken together, the above results indicate that enhanced rooting by exemplary conjugates is associated with a more complex root architecture, which may provide further advantages as a rooting enhancer.

### Example 4

### Auxin Activity of Exemplary Conjugates in Arabidopsis Model

The exemplary amino acid conjugates of 4-CPA (Compounds 1o-1t) were examined in an Arabidopsis model, a typical model for studying for evaluating auxin activity.

One technique for assessing auxin activity of the 4-CPA amino acid conjugates utilized plants expressing nucleus-localized, fluorescent DR5:venus marker, the expression level of which is an indicator of intracellular auxin activity [Laskowski et al., PLoS Biol 2008, 6:e307]. Four days old seedlings grown on regular MS medium were transferred to plates containing 10 μM of the tested compounds and fluorescence was inspected by confocal microscope after 24 hours.

As shown in FIGS. 14A and 14B, 4-CPA promoted fluorescence most strongly, as determined by fluorescent intensity (about 2-fold more potent than Compound 1t, the most potent conjugate); and Compounds 1o and 1s (which are conjugates of the D-isomer of valine and tryptophan, respectively) promoted significantly lower fluorescence of DR5 in comparison to their corresponding L-isomer conjugates, Compounds 1p and 1t, respectively.

These results indicate that L-amino acid conjugates have a more potent auxin activity than do their corresponding D-amino acid conjugates, which is consistent with the results obtained with a mung bean model as described hereinabove.

As further shown therein, the strong auxin activity of 4-CPA and the L-amino acid conjugates Compounds 1p and 1t) resulted in loss of auxin activity at the root tip, and swelling of the root elongation zone, suggesting imbalance in the natural auxin transport and feedback loops [Tanaka et al., Cell Mol Life Sci 2006, 63:2738-2754] under these conditions.

A common technique for evaluating auxin activity is by determining the ability of a compound to inhibit root elongation [Zolman et al., Genetics 2000, 156:1323-1337]. In order to use this assay for comparing auxin activities of different conjugates, the minimum concentration of free 4-CPA that is active in the root elongation inhibition assay was determined. Four days old seedlings were placed in vertical plates containing increasing concentrations of 4-CPA. The root lengths were marked daily during 5 days.

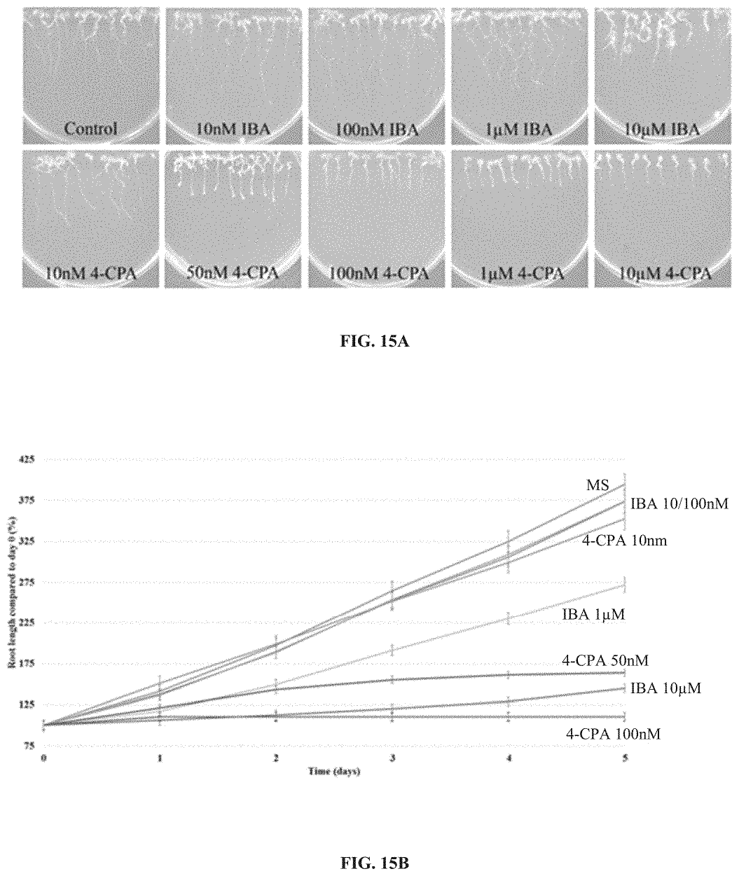

As shown in FIGS. 15A and 15B, an IBA concentration of 1-10 μM was required to inhibit root elongation, whereas 4-CPA inhibited root elongation significantly at 50 nM, and totally at 100 nM.

Based on the above results, the effect of conjugates of 4-CPA (Compounds 1o-1t) on root elongation after 5 days was determined at concentrations of 50 nM.

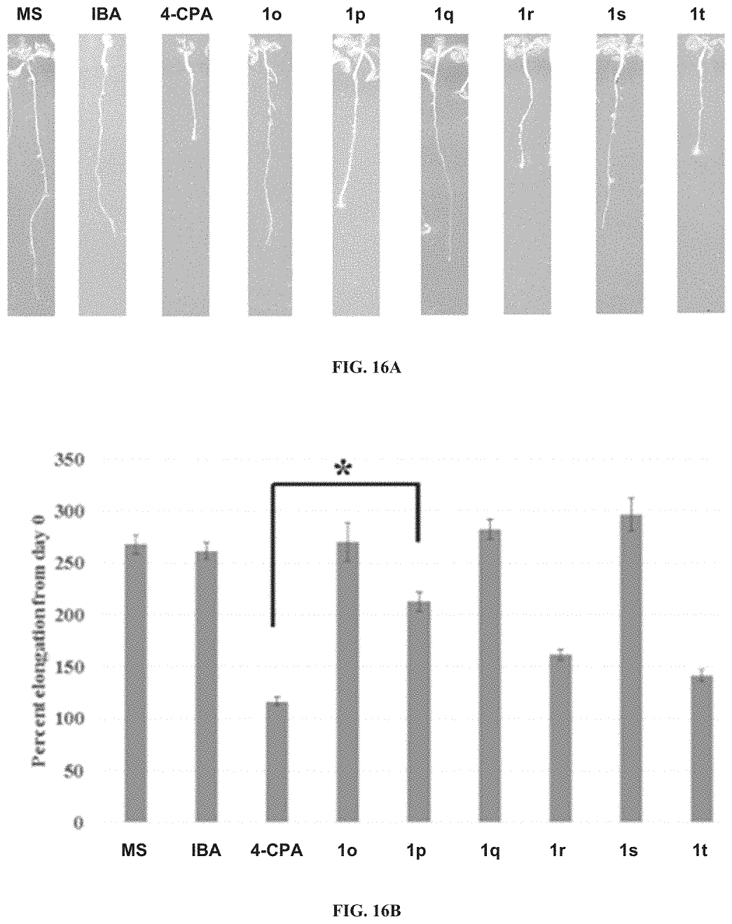

As shown in FIGS. 16A and 16B, the tested conjugates of 4-CPA (Compounds 1o-1t) exhibited a different effect on root elongation than did 4-CPA itself, and L-amino acid conjugates exhibited different effects than did their corresponding D-amino acid conjugates. The D-amino acid conjugates (Compounds 1o, 1q and 1s) did not affect root elongation (relative to control), whereas the L-amino acid conjugates (Compounds 1p, 1r and 1t) inhibited root elongation relative to control (with Compound 1t being the most active), albeit to a lesser extent than did free 4-CPA. Compound 1p was significantly (p<0.05) less inhibitory than 4-CPA. These results are consistent with those obtained with DR5 fluorescence, wherein conjugates with the L-amino acids had a stronger auxin activity than conjugates with the D-amino acids, and the L-Val conjugate (Compound 1p) had a weaker auxin activity than the other L-amino acid conjugates.

Interestingly, root elongation in the presence of the L-configuration Compounds 1p and it exhibited a biphasic behavior, unlike 4-CPA and IBA; a slight inhibition was observed until day 3, followed by an abrupt and complete inhibition starting on day 4.

This inhibition behavior suggests a gradual accumulation of auxin until a threshold concentration is reached that completely inhibits further growth consistent with slow release of the active compound.

As further shown in FIGS. 16A and 16B, IBA had no apparent inhibitory effect on root elongation. This result is consistent with the report that IBA is less potent than IAA in root elongation inhibition [Zolman et al., Genetics 2000, 156:1323-1337].

Taken together, these results these results suggest gradual release by the conjugates of a stable compound with auxin activity, which can explain the ability of such conjugates to enhance rooting of cuttings from mature Eucalyptus grandis trees (in which rooting is more difficult to induce than in Arabidopsis) to a greater extent than free 4-CPA and 4-CPA-glycine conjugate. These results further suggest that D-amino acid conjugates are hydrolyzed less rapidly than L-amino acid conjugates, and that L-Val conjugates are hydrolyzed less rapidly than L-Asp and L-Trp conjugates.

In a third assay utilizing an Arabidopsis model, induction of adventitious root AR formation in intact plants was examined in the presence of 50 nM of tested compounds, which included 5 days incubation in the dark, followed by a shift to the light, according to procedures described by Sorin et al. [Plant Cell 2005, 17:1343-1359].

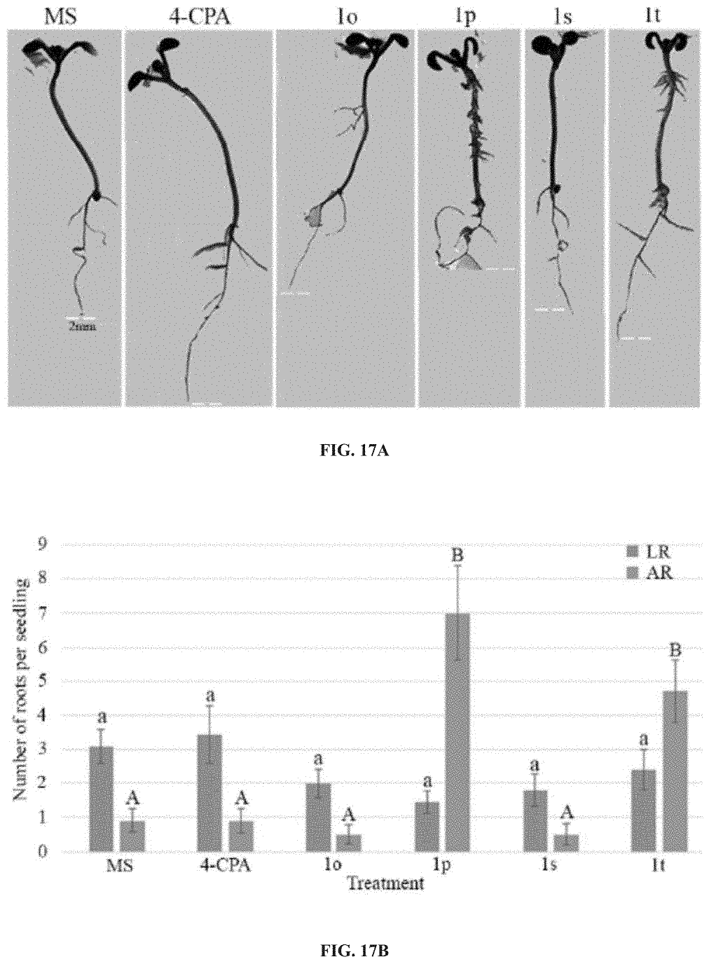

As shown in FIGS. 17A and 17B, Compounds 1p and 1t (L-amino acid conjugates) induced formation of 7±1.4 and 4.7±0.9 adventitious roots on average, respectively, whereas Compounds 1o and is (D-amino acid conjugates) induced formation of only 0.5±0.3 adventitious roots on average, and 4-CPA induced formation of only 0.8±0.4 adventitious roots on average.

The above experiment was repeated with additional 4-CPA conjugates, including the L- and D-isomers of phenylalanine (Phe), methionine (Met), glutamic acid (Glu) and threonine (Thr).

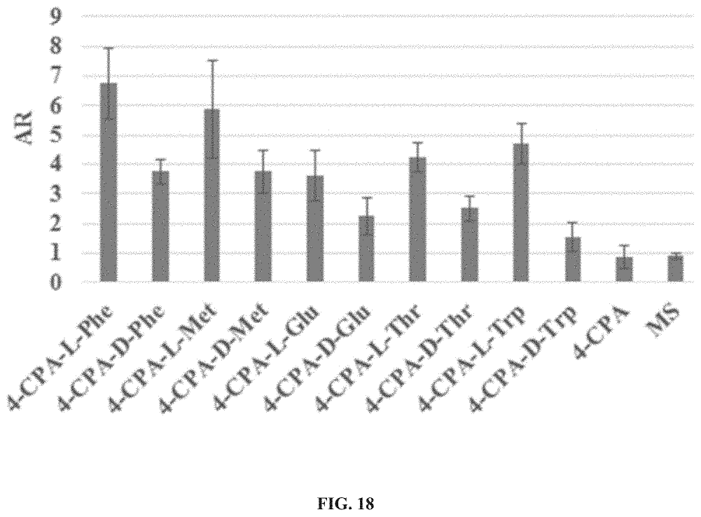

As shown in FIG. 18, a variety of amino acid conjugates of 4-CPA exhibited similar promotion of adventitious root formation in intact Arabidopsis, with L-amino acids consistently exhibiting more activity than the corresponding D-amino acids.

Taken together, the above results suggest that L-amino acids (and L-Val in particular) conjugates of 4-CPA provide an advantageous release rate of active 4-CPA.

### Example 5

### Effect of Conjugates on Root Formation in Argan and Jojoba Cuttings

The ability of exemplary conjugates to promote rooting in plants which are recalcitrant to rooting (in addition to the results obtained with Eucalyptus grandis in Example 3) was examined using cuttings of argan (Argania spinosa) and jojoba (Simmondsia chinensis) in a commercial nursery (Shorashim). The argan and jojoba cuttings were treated with 100 μM of a conjugate (any of Compounds 10-1t) with 6000 ppm IBA. The common treatment T-8 (talc with 8000 ppm IBA and a fungicide) or 6000 ppm IBA (without conjugate) served as a control.

As shown in FIGS. 19A and 19B, Compounds 1s and 1t resulted in the considerably higher rooting percentages in argan than did IBA alone.

As shown in FIGS. 20A and 20B, the exemplary conjugates usually resulted in more that 40% rooting of jojoba cuttings, and were considerably more effective than T-8 treatment, which resulted in less than 10% rooting in both plants.

These results indicate that conjugates described herein are effective at promoting rooting in a wide variety of recalcitrant plants.

### Example 6

### Effect of Conjugates on Root Formation in Avocado Cuttings

Avocado rootstock is extremely difficult to root. Currently, the common technique for propagating clones of avocado rootstock is to graft the desired rootstock on a seedling, and transfer to the dark to generate an etiolated branch. This branch can be rooted and grafted with the desired variety while still grafted on the seed [Frolich & Platt, California Avocado Society 1971-72 Yearbook 1972, 55:97-109]. However, the preparation of such twice-grafted seedlings is time-consuming and expensive.

As shown in FIG. 21, etiolated avocado branches rooted very effectively in the presence of IBA (at a rate of 80%), whereas green branches rooted considerably more poorly (at a rate of 10%).

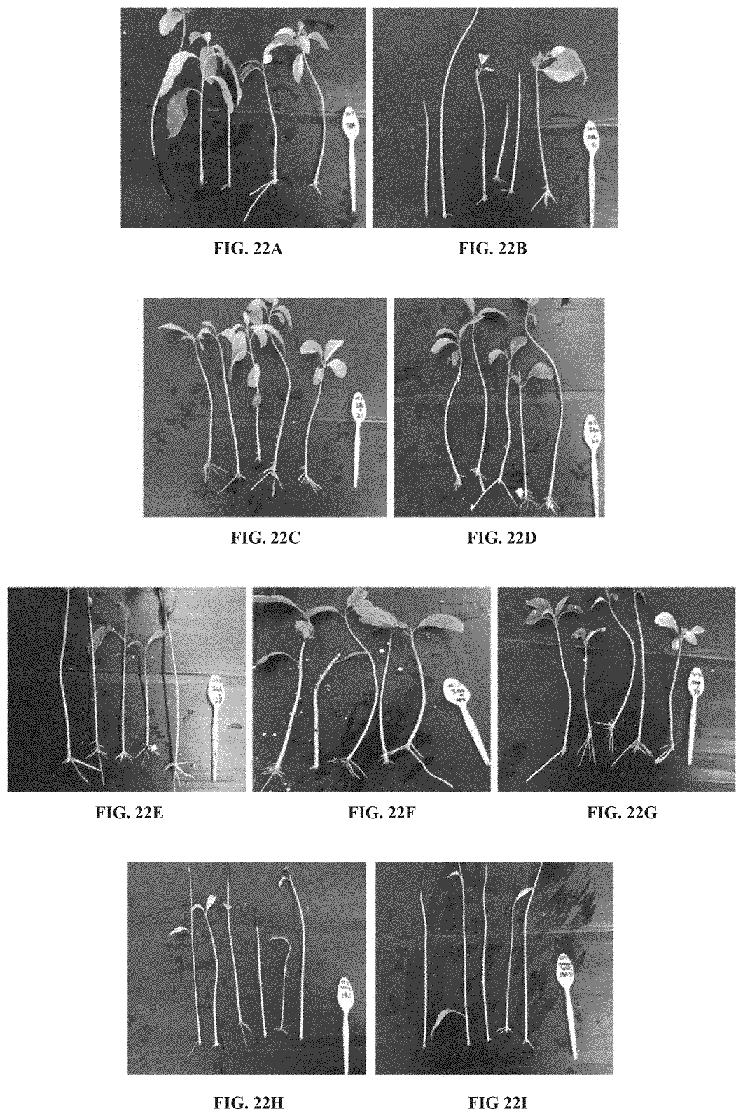

However, as the etiolated branches are more sensitive to pathogens and less resistant to rooting conditions, they are less suitable for rooting. Representative samples of etiolated branches and green branches are shown in FIGS. 22A-22I.

The ability of exemplary conjugates to promote rooting in green avocado cuttings (in the presence of IBA) was therefore examined. In particular, Compounds 2h (MCPA-methanol), 4b (NAA-β-alanine methyl ester), 3g (2-DP-methyl-4-aminobenzoate), 3f (2-DP-p-toluidine), 1l (4-CPA-morpholine) and is (4-CPA-D-Trp) were tested.

As further shown in FIG. 21, Compounds 1l, 1s, 2h, 3g, 3f and 4b considerably enhanced the rooting success rate of green avocado branches.

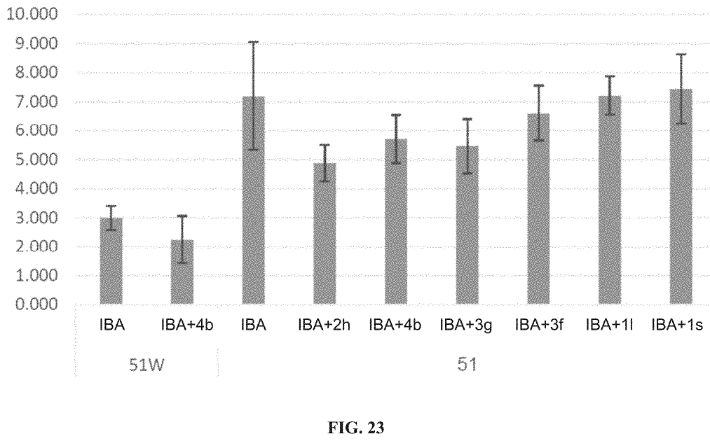

In addition, as shown in FIG. 23, green avocado branches treated with IBA or IBA and Compounds 1l, 1s, 2h, 3g, 3f and 4b exhibited considerably more roots per cutting than did etiolated avocado branches treated with IBA.

Successful rooting was obtained from various avocado rootstocks, and 21 saplings were obtained from VC801 rootstock, 15 from VC66 rootstock, 9 from Day rootstock, and 2 from VC804 rootstock.

Histological staining was performed in order to identify the source of the roots.

As shown in FIG. 24A, tracheary elements with apparently circular patterns of secondary cell wall thickening were clearly visible.

Similar structures have been reported at the junctions between trunk and branches [Lev-Yadun & Aloni, Trees 1990, 4:49-54] and at junctions where auxin transport from opposite directions meet [Sachs & Cohen, Differentiation 1982, 21:22-26].

As shown in FIG. 24B, differentiation of cork tissue was visible at the perimeter of the callus.

As shown in FIGS. 24C and 24D, cells rich in amyloplasts were abundant. FIG. 24D confirms that the visible organelles are amyloplasts, using polarized light microscopy.

These results indicate that the callus comprises different types of differentiated cells, and suggest that the roots originate from the callus which develops at the cutting base.

In additional experiments, avocado cuttings (etiolated and/or green) are treated with various exemplary conjugates and doses thereof, in order to characterize which treatments result in efficient rooting and which result primarily in callus formation.

### Example 7

### Effect of Conjugates on Root Development in Pine Cuttings

The effects of exemplary D-amino acid conjugates of 4-CPA (Compounds to and Is) on rooting in Pinus halepensis cuttings were compared with those of 2-DP-glycine methyl ester conjugate (2-DP-Gly). Rooting mature pine cuttings (i.e., cuttings that are taken from trees more than 4 years old) is very difficult.

Apical cuttings were taken from 7-year-old trees, stored for 4 weeks at 4° C., and treated by dipping the cutting bases for 4 hours in the following solution: 400 ppm IBA potassium salt+5 ppm tested conjugate+0.1% Amistar™ fungicide (250 grams/liter azoxystrobin). The cuttings were evaluated after 12 weeks.

As shown in Table 3 below, the tested compounds (in combination with IBA) all provided a high rooting rate, but the degree of root development was significantly higher with the D-amino acid conjugates of 4-CPA (Compounds to and Is) than with 2-DP-Gly.

These results indicate that exemplary 4-CPA conjugates can considerably enhance root system development in cuttings which are difficult to root. This phenomenon is important, as it is associated with enhanced development of rooted cuttings after transplantation to growing containers.

TABLE 3
Rooting rate and degree of root development in pine cuttings
treated with Compound 1o, Compound 1s, or 2-DP-Gly

| | Degree of root development | | | |
|---|---|---|---|---|
| | | | Well-developed | Rooting rate |
| | Weak (%) | Medium (%) | (%) | (%) |
| Compound 1o | 7.2 | 14.3 | 71.4* | 100 |
| Compound 1s | 6.2 | 0 | 81.3* | 92.9 |
| 2-DP-Gly | 46.7 | 6.6 | 46.7 | 87.5 |

(*indicates statistically significant difference from 2-DP-Gly treatment)

### Example 8

### Effect of Conjugates on Eucalyptus brachyphylla and Eucalyptus x trabutii Cuttings

Eucalyptus trees such as Eucalyptus x trabutii and Eucalyptus brachyphylla provide valuable sources of nectar and pollen for honeybees, especially during arid seasons when other food sources are in short supply. However, eucalyptus trees can exhibit a wide variety of blooming properties, which is believed to be at least in part because such trees are commonly grown from seeds, resulting in considerable genetic variability.

The ability to clone eucalyptus trees exhibiting particularly rich and/or constant blooming (resulting in progeny genetically identical to the parent) would therefore be advantageous, for example by inducing adventitious root formation in cuttings. An obstacle to such cloning is the loss of rooting ability upon maturation, before blooming traits are readily identifiable.

In order to investigate cloning of productive eucalyptus trees from cuttings, branches were excised from mature Eucalyptus x trabutii and Eucalyptus brachyphylla trees the field (in Kfar Pines, Israel)—selected based on their exceptional nectar production and honeybee attraction—placed in a humidified cooler box and brought to a climate-controlled greenhouse within 2 hours. Each cutting, 7 cm long, included three nodes with only two leaves remaining at its apical end. Two-thirds of each blade was excised to minimize evapotranspiration. The cutting bases were submerged for 1 minute in 6 grams/liter indole-3-butyric acid potassium salt (K-IBA), optionally supplemented with Compound 1s or 1t at a concentration of 100 μM. In addition, the leaves were sprayed with 100 μM of each compound in the presence of 0.05% Triton X-100 surfactant. Stock solutions of Compounds 1s and 1t were at a concentration of 100 mM in DMSO. The cuttings were planted in rooting medium containing peat, vermiculite and polystyrene flakes at a ratio of 1:2:3 respectively, on a heated rooting table under 90% humidity. Fungicides were applied to the rooting media on a weekly basis. Rooting percentage, number of roots per cutting and root length were measured after 1 and 2 weeks and after 1 and 2 months.

While rooting percentages in initial experiments were low (5-8%, data not shown), several mother plants were obtained in these experiments, and cuttings harvested from these mother plants (grown in a greenhouse) were used in subsequent experiments, with enhanced rooting rates.

As shown in FIGS. 25A and 25E, the rooting rates of cuttings of E. x trabutii and E. brachyphylla was up to about 45% following treatment with IBA and Compound 1s or 1t. The rooting rate was considerably higher than that obtained for E. x trabutii with IBA alone.

As shown in FIGS. 25B-25G, in E. x trabutii, Compounds 1s and 1t (in combination with IBA) had no apparent effect (relative to IBA alone) on mean root number per cutting (which was consistently about 5) or mean root length; whereas in E. brachyphylla, Compounds 1s and 1t enhanced mean root length (suggesting earlier root formation) and decreased mean root number (from about 4 to about 2).

The E. x trabutii and E. brachyphylla exhibited significantly different rooting kinetics. The E. x trabutii cuttings rooted relatively rapidly, after 1-2 weeks, but rooted cuttings had difficulty undergoing hardening and exhibited low survival rates (about 50%); whereas it took the E. brachyphylla cuttings 1-2 months to root, but survival of the rooted cuttings was close to 100%.

As further shown in FIGS. 25A and 25E, callus formation occurred frequently in E. brachyphylla upon rooting induction, but considerably more rarely in E. x trabutii (e.g., upon rooting induction with Compounds 1s and 1t, no calluses were observed in E. x trabutii). Such calluses often developed into a cylindrical shape resembling a root, an example of which is shown in FIG. 25D.

Without being bound by any particular theory, it is believed that the significant differences between the above results for E. brachyphylla and the results for E. brachyphylla described in Levy et al. [BMC Genomics 2014, 15:524] reflect the considerable genetic variability within E. brachyphylla, which is a hybrid of results involving E. kruseana and E. loxophleba [Grayling & Brooker, Aust J Bot 1996, 44:1-13], which are significantly different species.

The above results suggest that the nature of rooting enhancement effected by compounds described herein is affected by the nature of the obstacles to rooting in the absence of compounds. For example, rooting percentage is increased more in plants with low rooting percentages (e.g., the E. x trabutii described herein) than in plants with higher rooting percentages (e.g., the E. brachyphylla described herein), and the rate of root formation is increased more in plants with slow rooting (e.g., the E. brachyphylla described herein) than in plants with more rapid rooting (e.g., the E. x trabutii described herein).

### Example 9

### Translocation and Rate of Release of 4-CPA from Conjugates in Plant

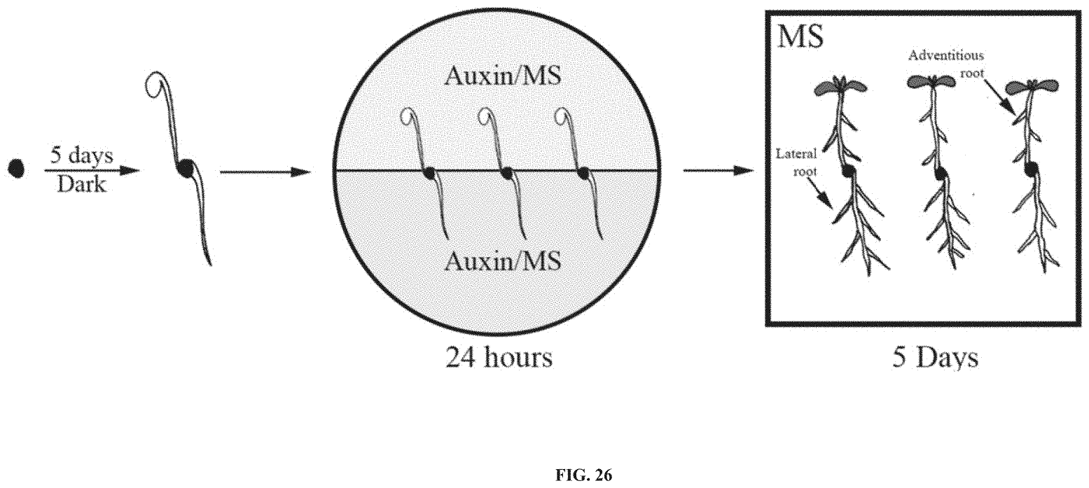

The observation that AR enhancement in Eucalyptus cuttings was higher when 4-CPA conjugates were applied also to the leaves (as described in Example 3) prompted further investigation into the functions of shoot-derived 4-CPA. To this end, 4-CPA or Compounds 1p and it were applied specifically to roots or shoots of five-day old etiolated Arabidopsis seedlings using a split petri dish (as depicted in FIG. 26) and the effect on root elongation, lateral root (LR) formation and adventitious root (AR) formation was evaluated.

Arabidopsis plants were germinated and grown in the dark for 5 days, then then transferred to petri dishes with a partition in the middle. Each half of these plates contained either medium alone or with 10 μM of 4-CPA or Compound 1p or 1tl. The etiolated seedlings were put on the plates with their collet region on the partition such that either the hypocotyl and cotyledons, or the root or both were in touch with the medium containing the tested compound. Adventitious roots and lateral roots were counted and the length of the primary root was measured after a week.

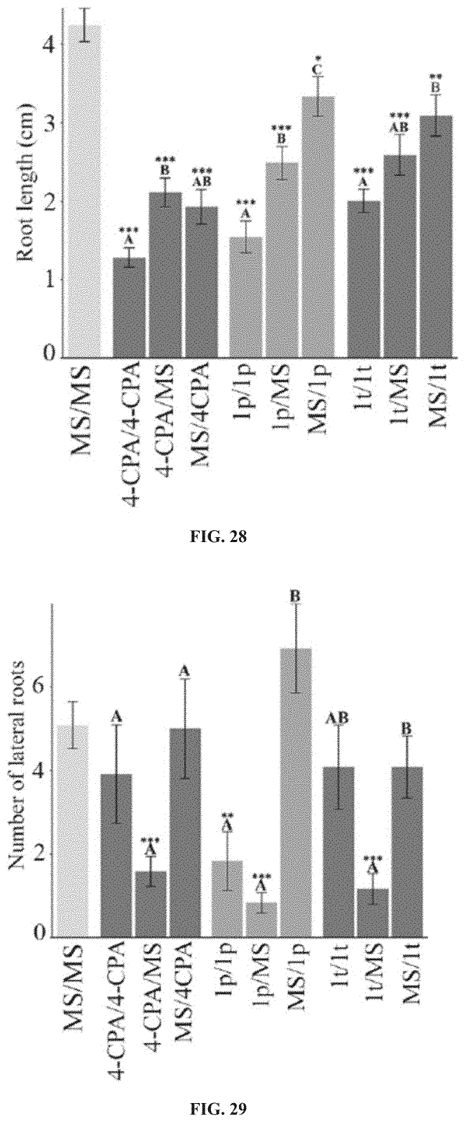

As shown in FIGS. 27 and 28, all treatments led to a significant inhibition of root elongation, with application to the shoot having comparable (for Compound 1t) or even stronger (Compound 1p) effect on root growth than application directly to the root.

Similarly, as shown in FIG. 29, shoot-applied 4-CPA or Compounds 1p or 1t inhibited lateral root formation more efficiently than application to roots.

These results indicate that there is efficient shoot-to-root translocation of 4-CPA or a response signal of 4-CPA.

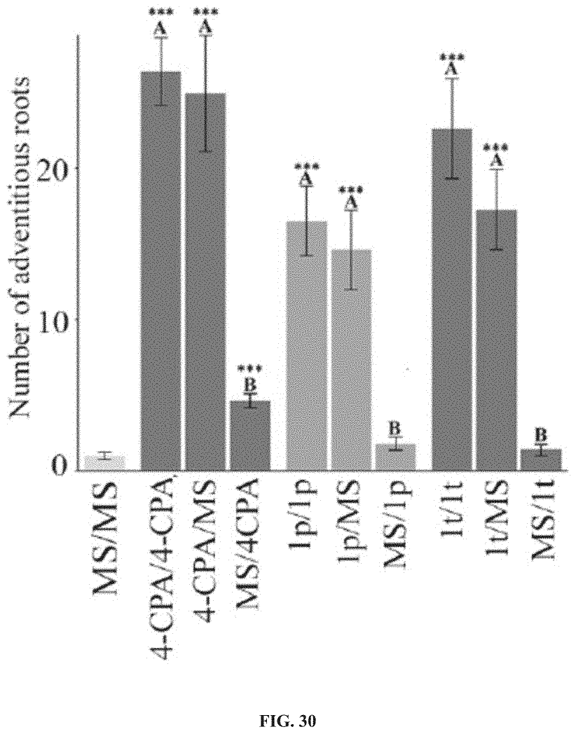

As shown in FIG. 30, exposure of shoots to 4-CPA or Compounds 1p or 1t resulted in significantly more adventitious root formation as compared to exposure of roots, suggesting that there is little or no root-to-shoot translocation of 4-CPA or response signal of 4-CPA.

The above results indicate that 4-CPA or conjugates thereof, when applied to shoots, had opposite effects on lateral and adventitious roots in Arabidopsis. This is somewhat surprising because it has been reported that IAA synthesized in the leaves is transported to the root and is required for lateral root emergence [Bhalerao et al., The Plant Journal 2002, 29:325-332], and that 2,4-D was able to block PIN1 endocytosis, prolong its stay in the membrane, and thereby regulate auxin efflux from the cells [Paciorek et al., Nature 2005, 435:1251-1256], which might suggest that 4-CPA is able to increase basipetal transport of auxin from the leaves to the root by doing the same. On the other hand, the auxin downstream signal transduction pathways promoting lateral and adventitious root formation have been reported to be different [Bellini et al., Annu Rev Plant Biol 2014, 65:639-666; Verstraeten et al., Front Plant Sci 2014, 5:495], and different synthetic auxins analogs have been shown to activate different sets of genes [Pufky et al., Funct Integr Genomics 2003, 3:135-143], thus raising the possibility that 4-CPA might have a different effect on lateral and adventitious roots than IAA.

Taken together, these results suggest that the higher rooting enhancement observed for shoot-applied conjugates is a result of their more efficient hydrolysis in this part of the plant, combined with effective basipetal movement of the released 4-CPA or response signal.

In order to determine whether trend observed in Arabidopsis occurs also in eucalyptus cuttings, the 4-CPA translocation directionality was first examined by treating cuttings with 4-CPA (100 μM) either by submergence of the cutting base or by spraying the leaves. At several time points (i.e. 0, 1, 6 and 24 hours), the cutting base (bottom 2 cm) and the leaves were separately extracted (with isopropanol/methanol/acetic acid solution) and 4-CPA levels were determined by HPLC-MS/MS.

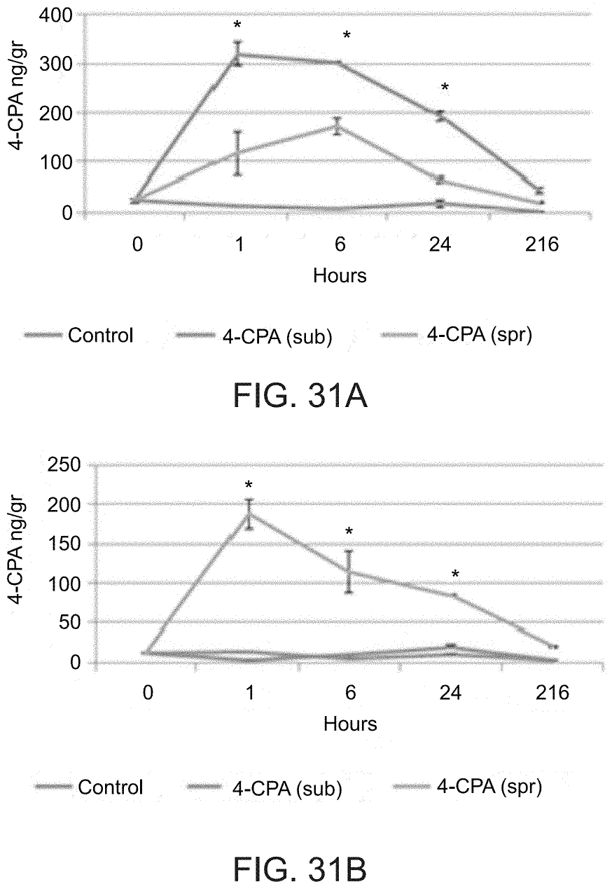

As shown in FIGS. 31A and 31B, when 4-CPA was applied to the cutting base, 4-CPA accumulated locally (about 275 ng/gram) with only trace amounts in leaves (less than 10 ng/gram); whereas when applied to leaves, 4-CPA was found both locally and in the cutting base at comparable levels (about 150 ng/gram). As further shown therein, whereas 4-CPA levels peaked locally within 1 hour from application, it was only after 6 hours that levels peaked following application to the leaves.

These results are consistent with those obtained in Arabidopsis (FIGS. 27-30).

In order to evaluate the rate of conjugates hydrolysis, 4-CPA levels were monitored following treatment with Compounds 1s and 1t. Cuttings were treated with IBA alone (by submerging the cutting base), or with IBA and Compound 1s or it by submerging the cutting base and by spraying the leaves with Compound 1s or 1t. Cutting bases (bottom 2 cm) and leaves were separately extracted 6, 24 and 48 hours after treatment and hormones levels were determined by HPLC-MS/MS.

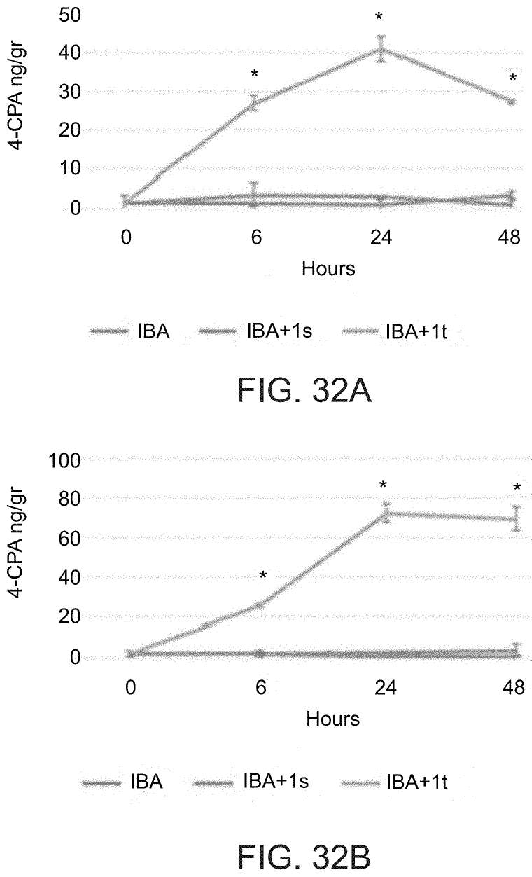

As shown in FIGS. 32A and 32B, 4-CPA levels were higher in cuttings treated with Compound 1t (40 ng/gram in the base and 70 ng/gram in the leaves) as compared to Compound 1s (close to zero throughout the measurements).

As the physical and chemical properties of Compounds 1s and 1t are similar, these results indicate that enzymatic hydrolysis is involved in the more rapid hydrolysis of Compound 1t. The higher levels of 4-CPA observed in leaves (as compared to cutting base) suggest increased absorbance due to increased surface area and/or due to increased hydrolytic activity in this organ. 4-CPA levels following conjugate treatment (up to 70 ng/gram) were significantly lower than those observed upon treatment with free 4-CPA (250-300 ng/gram) throughout the measurements; but conjugate treatment led to continuously increasing 4-CPA levels whereas 4-CPA levels peaked after 1 hour upon treatment with free 4-CPA, indicating that 4-CPA is released gradually from the conjugates.

In addition, endogenous auxins and their natural metabolites were determined following treatment with IBA alone or with Compound 1t.

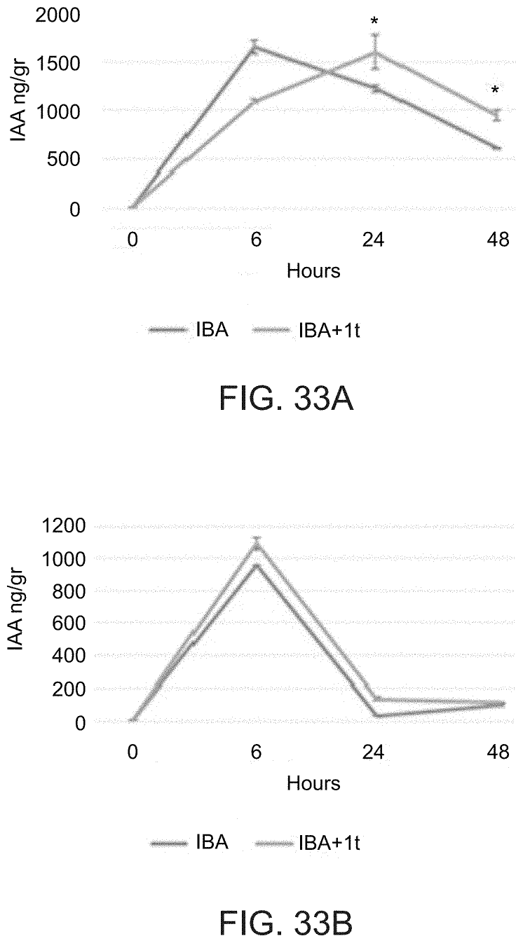

As shown in FIG. 33A, indoleacetic acid (IAA) levels in the cutting base peaked 6 hours after treatment with IBA but only after 24 hours after treatment with IBA and Compound 1t.

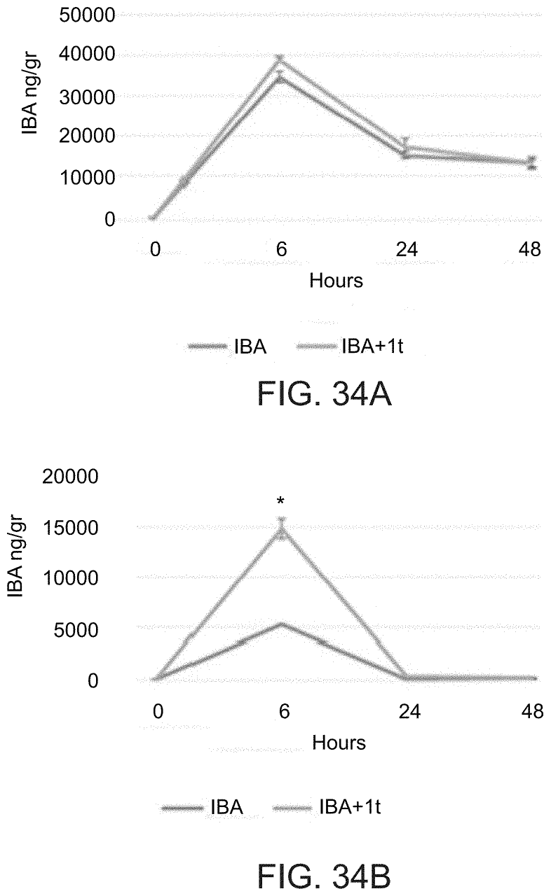

As shown in FIGS. 34A and 34B, IBA levels peaked 6 hours after treatment with either IBA alone or IBA and Compound 1t, but the use of Compound 1t resulted in a significant increase in IBA levels in leaves (15 ng/gram with Compound 1t versus 5 ng/gram with IBA alone).

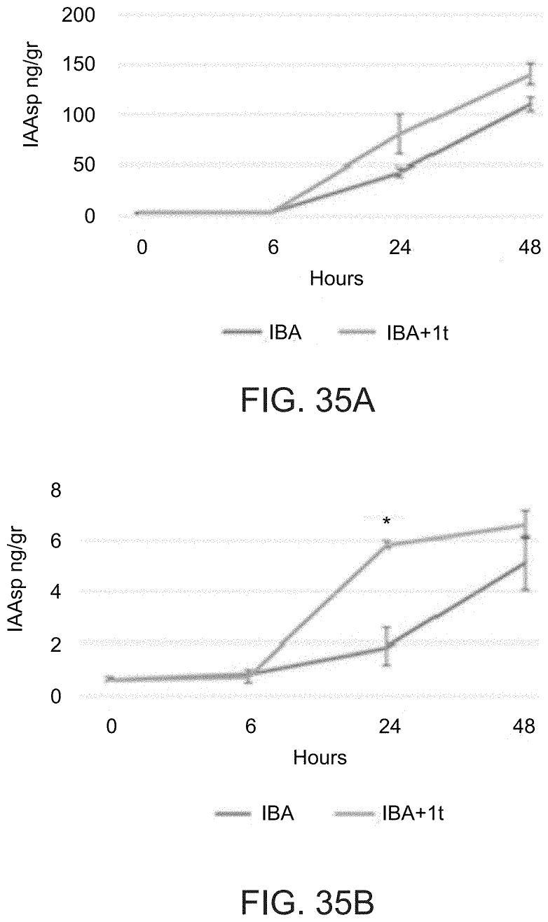

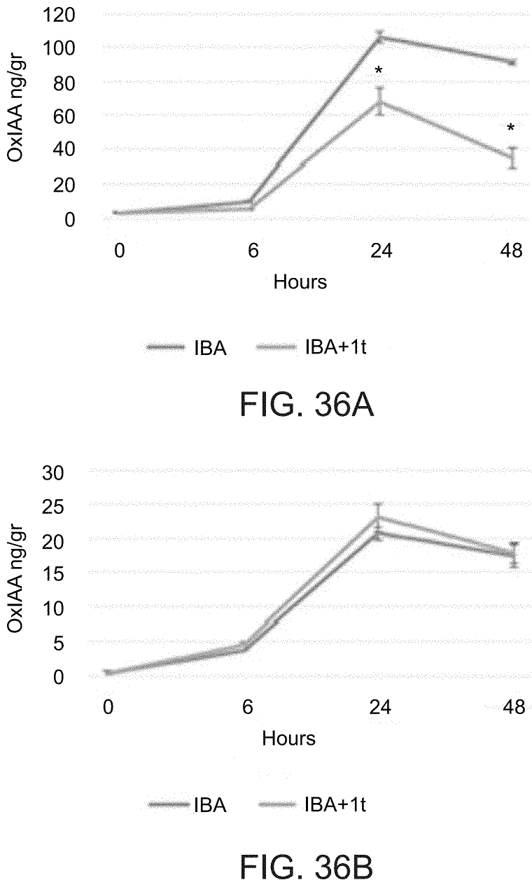

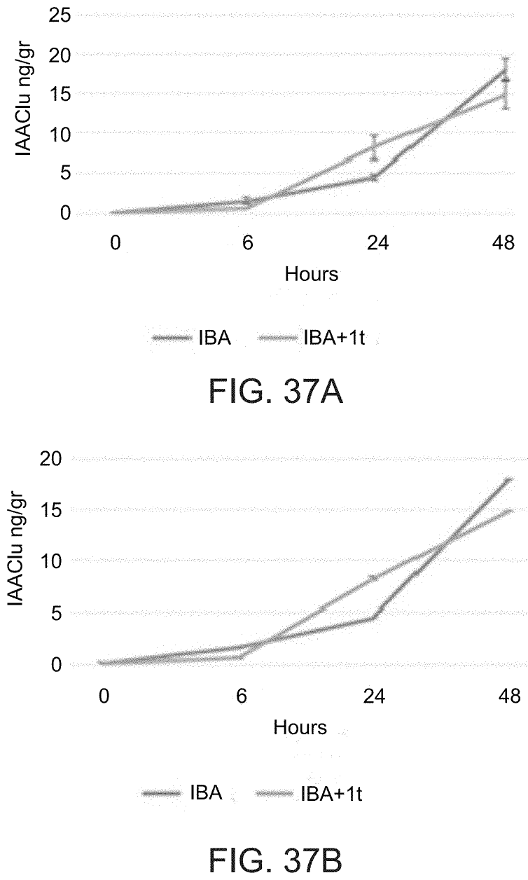

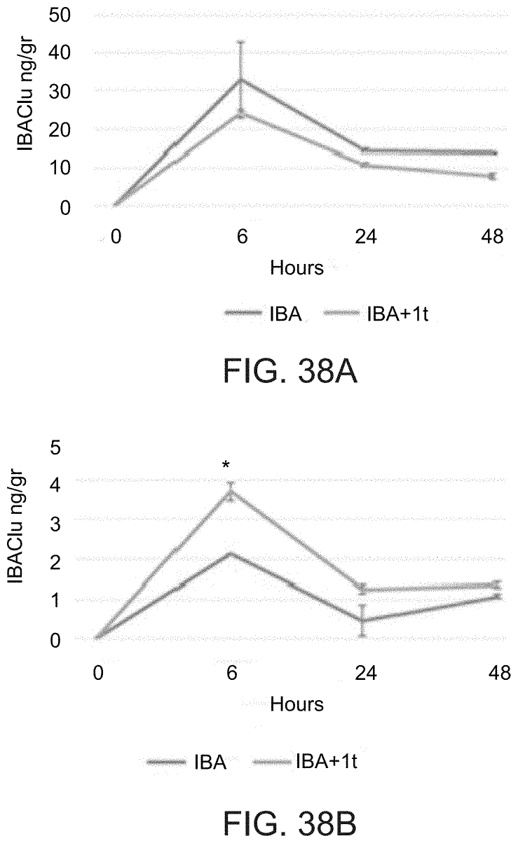

As shown in FIGS. 35A-38B, natural conjugates IAA-Asp (FIGS. 35A and 35B), IAA-Glu (FIGS. 37A and 37B) and IBA-Glu (FIGS. 38A and 38B) accumulated in greater or equal amounts upon treatment with IBA and Compound 1t than with IBA alone; and more 2-oxindole-3-acetic acid (FIGS. 36A and 36B), a largely inactive degradation product of IAA, accumulated in the cutting base upon treatment with IBA.

As IBA has been reported to be a precursor of IAA [Strader et al., Plant Physiol 2010, 153:1577-1586] and IBA-Glu may be a storage form of auxin [Korasick et al., J Exp Biol 2013, 64:2541-2555], these results suggest that IBA-Glu accumulation after 6 hours in the leaves may contribute to higher IAA levels in the cutting base after 24 hours.

Taken together, the above results suggest that increased hydrolysis of the conjugate in the leaves, combined with the basipetal transport of 4-CPA may underlie the advantage presented by the combined treatment described herein (application to both leaves and cutting base) in promoting root formation. Such a mechanism may provide a lengthy supply of auxin for basipetal transport towards the cutting base, in which auxin accumulation is important for adventitious root formation [Druege et al., Front Plant Sci 2016, 7:381].

### Example 10

### Effect of Exemplary Conjugate on Gene Expression in Eucalyptus Model

In order to evaluate the effect of conjugates on signaling pathways, changes in expression profiles in response to IBA or IBA+Compound 1t were examined. In Eucalyptus grandis the origin of adventitious roots is from the cambium tissue [Abu-Abied et al., BMC Genomics 2014, 15:826]. Therefore, the expression analysis was based on the cutting bases, a cell fraction enriched with cambium (as shown in FIGS. 39A-39C).

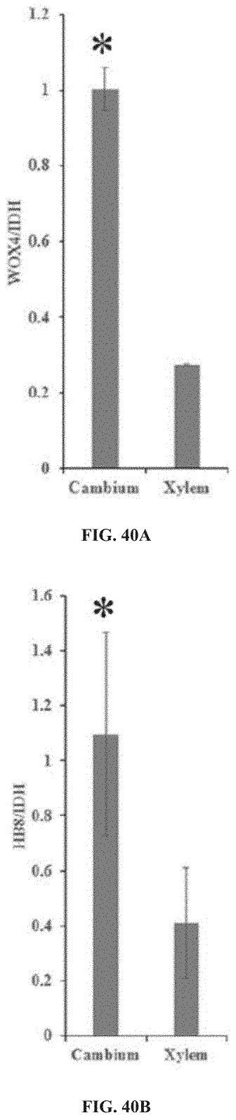

As shown in FIGS. 40A and 40B, enrichment in cambium cells was confirmed using a real time PCR assay performed using the specific cambium markers WOX4 (FIG. 40A) and HB8 (FIG. 40B), according to procedures described by Oles et al. [PLoS One 2017, 12:e0171927].

RNA-sequencing was performed in three replicates for the two time points: 0 before any treatment, and 24 hours following two treatments, IBA alone or IBA+Compound 1t (wherein Compound 1t was applied by both submersion and spraying, as described hereinabove). The time point of 24 hours was chosen because of the peak of 4-CPA accumulation in the leaves and cutting bases (e.g., as shown in FIGS. 32A and 32B) and the notable IAA accumulation in the cutting bases (e.g., as shown in FIGS. 33A and 33B) at this time.

As shown in Table 4 below, the reads, 17.5-23.6×10⁶ exhibited 87.2-89.6% mapping to the Phytosome E. grandis genome v2.

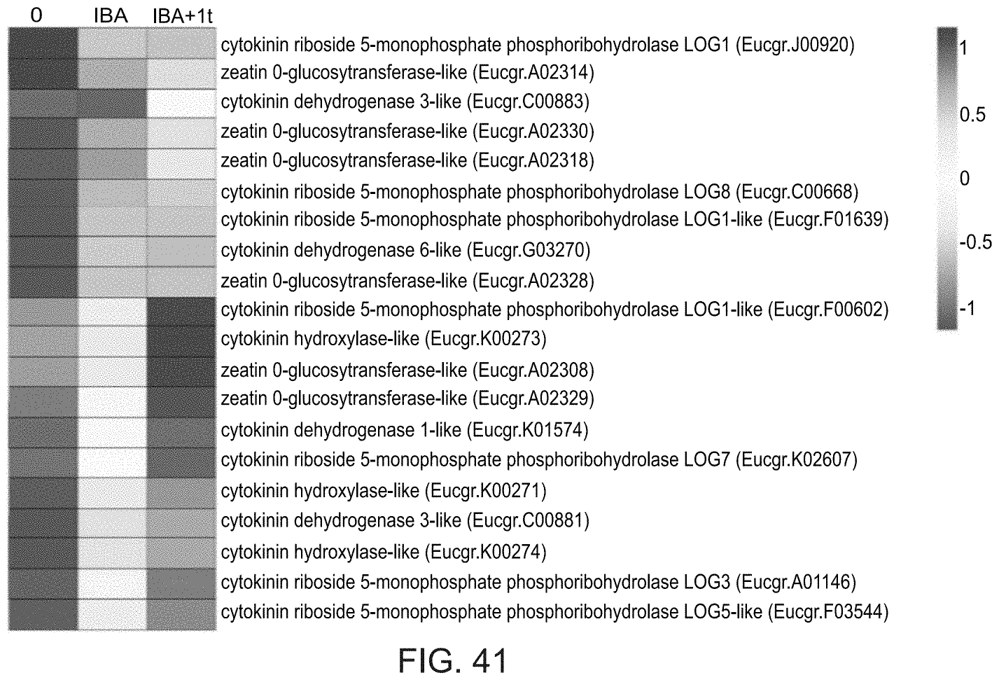

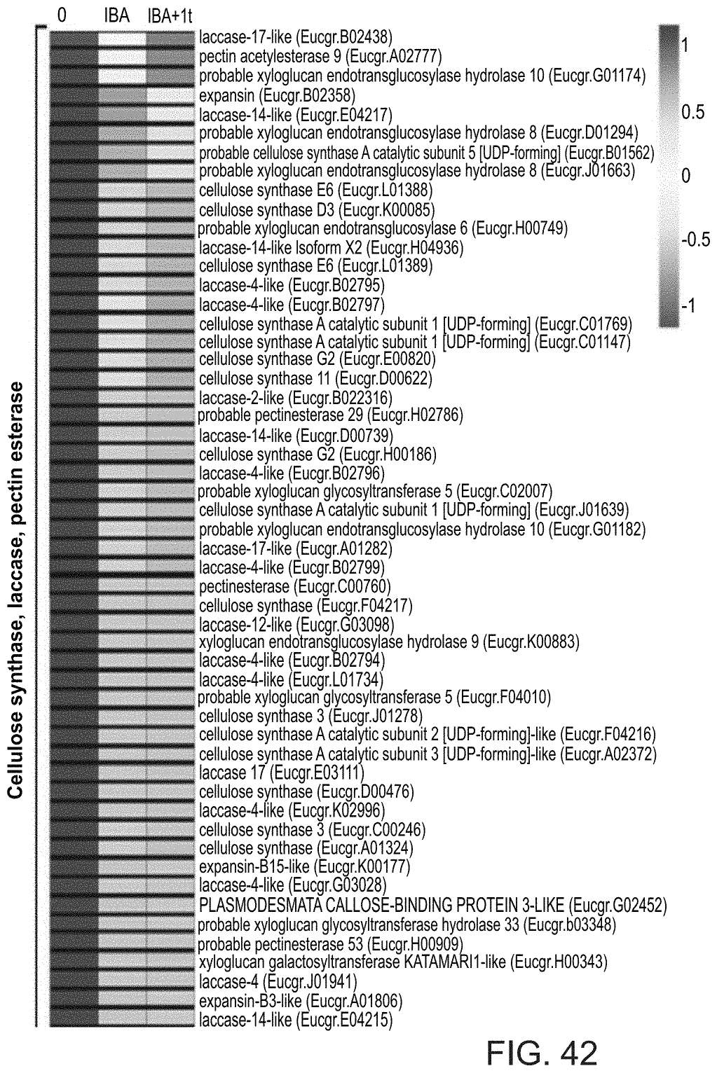

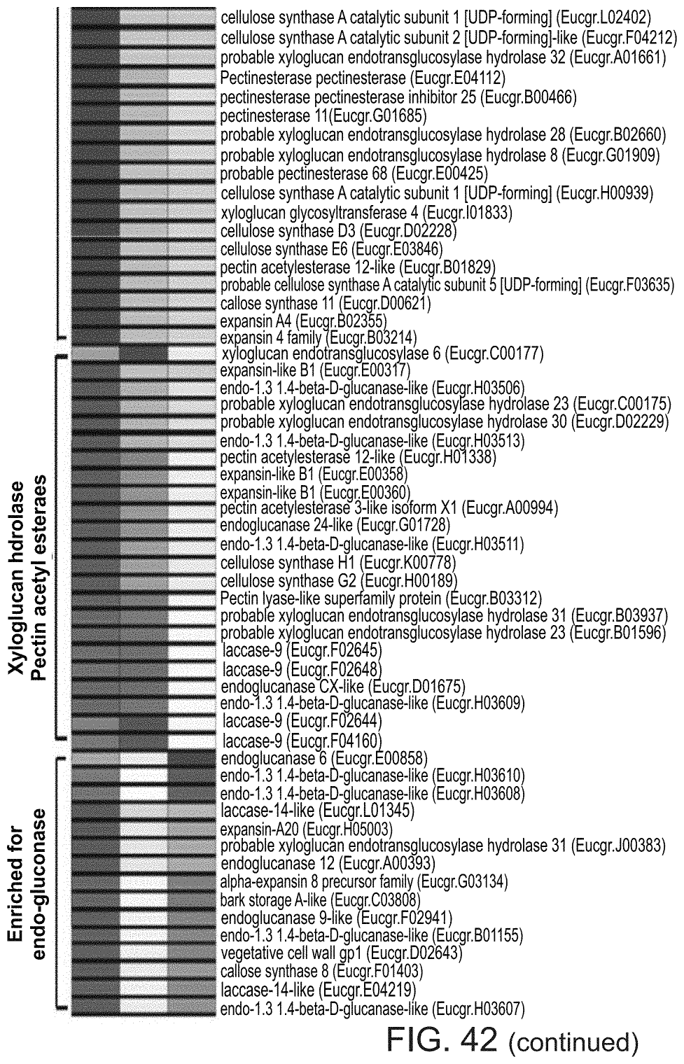

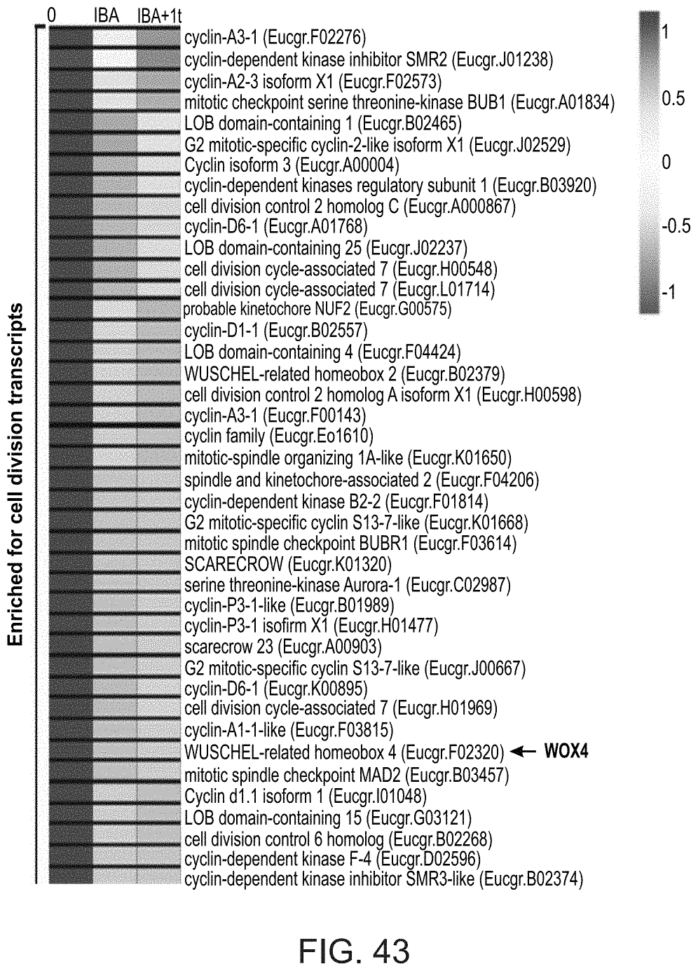

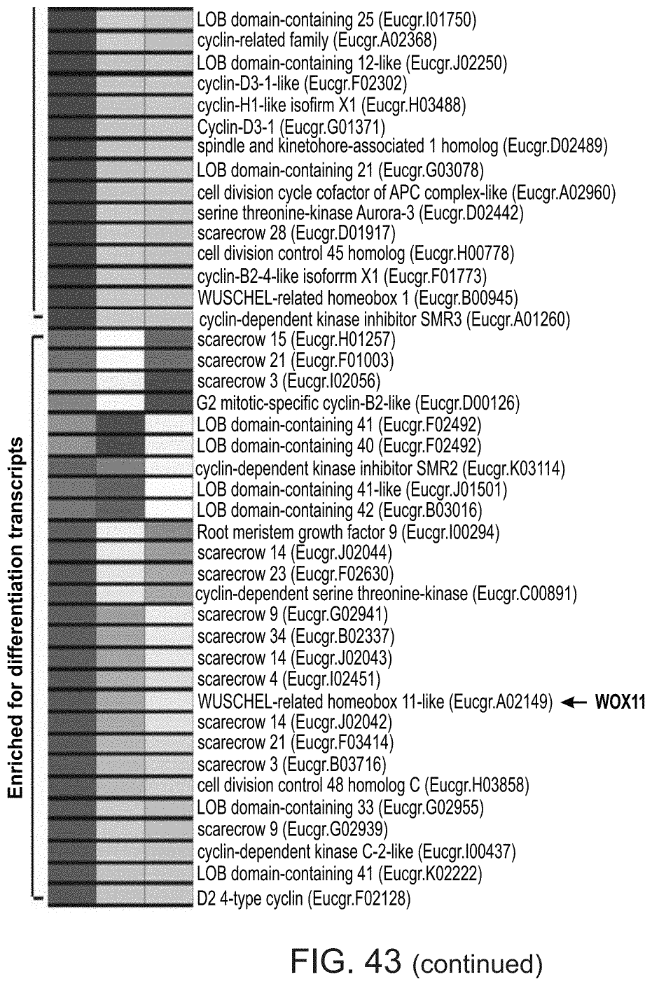

1924 transcripts were differently regulated (>two fold, adj p<0.05) between time 0 and MA or time 0 and IBA+Compound 1t, and 82 transcripts were differently expressed when comparing IBA to IBA+Compound 1t. Out of these, transcripts related to four functional groups were selected: auxin, cytokinin (as shown in FIG. 41), the cell wall (as shown in FIG. 42), and cell division and meristematic differentiation (as shown in FIG. 43).

TABLE 4
Clean reads upon RNA sequencing and mapping to E. grandis genome

| | | % mapping vs. Phytosome |
|---|---|---|
| Sample | Clean reads | (E.grandis_297_v2.0) |
| Time 0 | 23,138,775 | 88.1 |
| | 20,797,280 | 87.9 |
| | 19,764,641 | 88 |
| IBA | 22,772,010 | 87.2 |
| | 24,776,091 | 87.5 |
| | 17,520,226 | 88.1 |
| IBA + Compound 1t | 19,308,866 | 87.9 |
| | 18,644,900 | 89.6 |
| | 23,608,969 | 86.6 |

The transcripts related to auxin included those involved with auxin metabolism such as YUCCA and tryptophan aminotransferase-like transcripts, the expression of which was high at time 0 and decrease dramatically after IBA or IBA+Compound 1t treatments.

These results suggest a local auxin synthesis that is reduced in the presence of high ectopic auxin. Other genes belong to families of auxin conjugating enzymes, conjugate hydrolyzing enzymes, auxin transport and auxin responsive genes that underlie the regulation of specific auxin homeostasis and signaling which was slightly different between the two treatments.

Cytokinin homeostasis related transcripts were also found, some of which are involved with cytokinin activation such as LOG enzymes and hydroxylases, and others in reversible inactivation such as O-glucosyltransferases, or permanent inactivation such as dehydrogenases [Kieber & Schaller, Development 2018, 145:149344]. The spatiotemporal activity of cytokinin has been reported to be important for lateral root differentiation and growth [Jing & Strader, Int J Mol Sci 2019, 20:E486],

Co-reduction was observed of expression of cell wall-related transcripts corresponding to cellulose synthase complex, laccase (lignin synthesis) and pectin esterase in parallel to expression of transcripts related to cell division such as cyclins, cyclin dependent kinases and spindle checkpoint proteins, as well as that of WOX4, characterizing cambium meristematic cells. At the same time, co-induction was observed of expression of transcripts corresponding to cell wall modifying enzymes such as xyloglucan hydrolases, pectin acetyl esterases and endoglucanase.

In addition, upregulation was observed of transcripts related to differentiation, such as scarecrow, LOB domain proteins, and WOX11, characterizing AR founder cells. Scarecrow was found to be correlated with adventitious root formation in other trees [Sanchez et al., Tree Physiol 2007, 27:1459-1470; Stevens et al., Tree Physiol 2018, 38:877-894; Vielba et al., Tree Physiol 2011, 31:1152-1160], and WOX11 is expressed in AR founder cells in Arabidopsis and rice [Hu & Xu, Plant Physiol 2016, 172:2363-2373; Liu et al., Plant Cell 2014, 26:1081-1093; Zhang et al., Front Plant Sci 2018, 9:523].

The above results suggest cell wall modifications that accompanied the changes in cambium cell fate.

Taken together, the above results indicate that Compound 1t may promote root formation via small changes in several systems in parallel, which may together create more permissive conditions for adventitious differentiation.

### Example 11

### Conjugates with Enhanced Water Solubility

Additional conjugates were prepared according to procedures such as described in Example 1 hereinabove, except that the carboxylic acid ester (of an amino acid) was replaced by a more hydrophilic moiety.

In one general procedure, a conjugate comprising carboxylic acid ester is prepared as described hereinabove, and then hydrolyzed by contact with a strong base, such as NaOH, KOH, LiOH, etc., thereby resulting in a conjugate comprising a free carboxylic acid group or a salt (e.g., alkali metal salt) thereof.

In exemplary embodiments, a conjugate of 4-CPA and an amino acid methyl ester was added to 3 ml methanol in a 10 ml process vial equipped with a stirring bar, followed by addition of an aqueous solution of sodium hydroxide (3 equivalents for a mono-ester and 6 equivalents for a di-ester) in 1 ml water. The vial was fitted with a snap-on cap, inserted to a Discover™ SP microwave and stirred for 10 seconds under the following conditions: temperature 90° C., power 100 W, hold time 10 minutes, no pre-mix, high stirring, cooling on. The solution was transferred to a 20 ml vial and evaporated by a V-10 system. The obtained crude was dissolved in water (5 ml) and the pH adjusted to 3 with 2 N HCl. Upon completion of precipitation of the product, the solid was filtered and washed with water, and the obtained solid lyophilized overnight. A stoichiometric amount of NaOH in 5 ml water was then added and the solid lyophilized, to obtain the final product as a sodium salt.

Using the above general procedures, the following conjugates were obtained (purity was determined by HPLC at a wavelength of 254 nm):

Compound 82: 4-CPA-L-Asp disodium salt (molecular weight 345.64, purity >95%)

Compound 83: 4-CPA-L-Val sodium salt (molecular weight 307.1, purity >95%)

Compound 84: 4-CPA-L-Trp sodium salt (molecular weight 394.79, purity >95%)

In order to assess the ability of the abovementioned sodium salts to penetrate the cells and activate an auxin response, an Arabidopsis DR5-Venus model was utilized (as described hereinabove). The plants were exposed to 10 μM of each compound for 24 hour; MS medium was used as a negative control, and the related Compounds 1p, 1r and 1t were used as positive controls.

As shown in FIGS. 44A and 44B, that although 4-CPA-L-Asp disodium salt did not promote DR5 activity, 4-CPA-L-Val and 4-CPA-L-Trp sodium salts were about as active as their non-water soluble methyl esters (Compounds 1p and 1t, respectively).

Cannabis is usually an easy to root plant. However, some elite clones exhibit a certain degree of rooting difficulty. A relatively difficult to root cannabis clone was treated with 6000 ppm IBA or with a similar treatment combined with 50 μM of Compound 82 (4-CPA-L-Trp sodium salt) for 1 minute. Rooting was scored after 2 weeks.

As shown in FIGS. 45A and 45B, Compound 82 resulted in a significant increase in number of roots in cannabis, and in a small increase in the rooting percentage (which was already relatively high even in the absence of Compound 82).

Compound 82 was also used to induce rooting of transgenic citrus rootstocks in tissue culture. It is well known that shoots created in tissue culture conditions following genetic modifications are typically difficult to root. Using Compound 82, 60% rooting was obtained, as compared to 0% without Compound 82 (data not shown).

These results indicate that sodium salt conjugates such as described herein may be effective rooting enhancers in a variety of plants, including plants associated with tissue culture conditions.

### Example 12

### Additional Conjugates of 4-CPA and Amino Acids

An optimized general procedure for preparing conjugates of 4-CPA and amino acids (e.g., in the form of amino acid esters) was developed as follows:

In a first step (pre-activation), 4-CPA (1 equivalent) and CDI (1.2 equivalent) in 2 ml DMSO (2.5 mmol scale of 4-CPA in DMSO) were placed into a 10 ml process vial equipped with a stirring bar. The solution was stirred for 10 seconds and then the vial was fitted with a snap-on cap and inserted to a Discover™ SP microwave (90° C., power 100 W, 5 minutes).

In the second step (amide bond formation), a solution of amino acid methyl ester hydrochloride (1.2 equivalent) in 2 ml DMSO (3 mmol scale of amino acid in DMSO) was added to the reactor vial at room temperature. Then triethylamine (2.5 equivalents) was added quickly and the solution was irradiated for another 5 minutes under the same microwave conditions described hereinabove. The obtained crude was absorbed to FastWoRX™-S sorbent powder and the solvents were evaporated under reduced pressure. Purification was performed by Isolera™ flash system using a Biotage® SNAP 60 gram column (linear gradient from 10-100% acetonitrile).

Using the above general procedures, 27 conjugates have been synthesized (as shown in Table 5 below), which in addition to the 6 amino acid conjugates described hereinabove, result in thorough coverage of the 39 possible natural amino acids (including D- and L-amino acids).

TABLE 5
Conjugates synthesized according to
some embodiments of the invention

| | Quantity (mg) |
|---|---|
| Conjugate | (at purity > 95%) |
| 4-CPA-L-Ile-methyl ester | 444 |
| 4-CPA-L-Phe-methyl ester | 634 |
| 4-CPA-D-Phe-methyl ester | 548 |
| 4-CPA-L-Pro-methyl ester | 583 |
| 4-CPA-D-Pro-methyl ester | 479 |
| 4-CPA-L-Leu-methyl ester | 272 |
| 4-CPA-D-Leu-methyl ester | 552 |
| 4-CPA-L-Ala-methyl ester | 362 |
| 4-CPA-D-Ala-methyl ester | 507 |
| 4-CPA-Gly-methyl ester | 488 |
| 4-CPA-L-His-methyl ester | 340 |
| 4-CPA-L-Leu-methyl ester | 274 |
| 4-CPA-L-Met-methyl ester | 351 |
| 4-CPA-D-Met-methyl ester | 307 |
| 4-CPA-L-Asn-methyl ester | 331 |
| 4-CPA-L-Asp-methyl diester | 505 |
| 4-CPA-D-Asp-methyl diester | 441 |
| 4-CPA-L-Glu-methyl diester | 539 |
| 4-CPA-D-Glu-methyl diester | 518 |
| 4-CPA-L-Ser-methyl ester | 457 |
| 4-CPA-D-Ser-methyl ester | 694 |
| 4-CPA-L-Tyr-methyl ester | 800 |
| 4-CPA-L-Thr-methyl ester | 760 |
| 4-CPA-D-Thr-methyl ester | 774 |
| 4-CPA-L-Arg-methyl ester | |
| 4-CPA-L-Lys-methyl ester | |
| 4-CPA-D-Lys-methyl ester | |

Conjugates prepared as described herein were evaluated in a Eucalyptus grandis rooting model, according to procedures such as described hereinabove. The bases of cuttings (a 12-15 cm long branch with the two upper leaves, whose blades were cut in half) were dipped in a solution containing 6000 ppm IBA potassium salt and 50 μM of conjugate for 1 minute, and the leaves were sprayed with 50 μM of the same conjugate (mixed with 0.5% Triton™ X-100 surfactant). The cuttings were imbedded in a heated (25° C.) rooting table containing a 1:2:3 ratio of peat:vermiculite:polystyrene, under 90% relative humidity. Cuttings were evaluated after 1 month, and cuttings which did not root were left for another month.

The conjugates 4-CPA-L-Ile-methyl ester, 4-CPA-L-Pro-methyl ester, 4-CPA-L-Leu-methyl ester, 4-CPA-L-His-methyl ester and 4-CPA-L-Asn-methyl ester each promoted rooting in eucalyptus cuttings at a percentage in a range of from 25 to 36%. In addition, 4-CPA-L-Ile-methyl ester and 4-CPA-L-Leu-methyl ester both promoted an increase in root number (as compared to IBA control). The D-isomers 4-CPA-D-Pro-methyl ester and 4-CPA-D-Leu-methyl ester promoted a lower rate of rooting (10% and 5%, respectively), which is consistent with results presented hereinabove showing lower potency of D-amino acid conjugates.

The above experiment was repeated in a different season of the year, using 4-CPA-L-Ile-methyl ester, 4-CPA-L-Pro-methyl ester, 4-CPA-L-Leu-methyl ester, 4-CPA-L-His-methyl ester and 4-CPA-L-Asn-methyl ester.

In the repeated experiment, 4-CPA-L-Ile-methyl ester was particularly effective, resulting in 50% rooting of cuttings (as opposed to 10% for the IBA control).

Conjugates prepared as described herein were evaluated in an Arabidopsis rooting model, according to procedures such as described hereinabove. Seeds were germinated and kept in the dark for 4 days. The etiolated seedlings were then incubated for 1 hour in 10 μM of each compound and then transferred to vertical MS medium-containing plates.

As shown in FIG. 46, all the tested conjugates enhanced the number of adventitious roots, with 4-CPA-L-Leu-methyl ester being particularly potent (resulting in an average of 10 adventitious roots).

It is notable that 4-CPA-L-Leu-methyl ester was highly effective at inducing rooting in both the Arabidopsis and the eucalyptus model.

Exemplary conjugates were also tested for rooting the hard-to-root species of avocado (VC801 rootstock) and argan, according to procedures described hereinabove.

Treatment of VC801 avocado cuttings with 4-CPA-L-Ile-methyl ester resulted in 60% rooting, as compared to about 35% for the IBA control. The root architecture was also improved, with more roots per cutting and considerably longer roots, as compared the IBA control.

Treatment of VC801 avocado cuttings with Compound 82, 4-CPA-L-Leu-methyl ester, 4-CPA-L-His-methyl ester and 4-CPA-L-Asn-methyl ester also considerably increased root length, as compared to the IBA control.

Argan clone YM3 is relatively easy to root, especially in comparison to other argan clones.

After 1.5 months, 80% rooting in argan clone YM3 was obtained upon treatment with Compound 82, as compared with 50% in the IBA control. In addition, the root system architecture was improved, with about 7 roots per cutting, as compared to 2-3 for the IBA control.

Treatment of argan clone YM3 cuttings with 4-CPA-L-Ile-methyl ester, 4-CPA-L-Leu-methyl ester, 4-CPA-L-His-methyl ester and 4-CPA-L-Asn-methyl ester also considerably increased the number of roots per cutting, as compared to the IBA control.

The above results indicate that a wide variety of exemplary conjugates may be used in diverse array of plants.

### Example 13

### Additional Conjugates with Enhanced Water Solubility

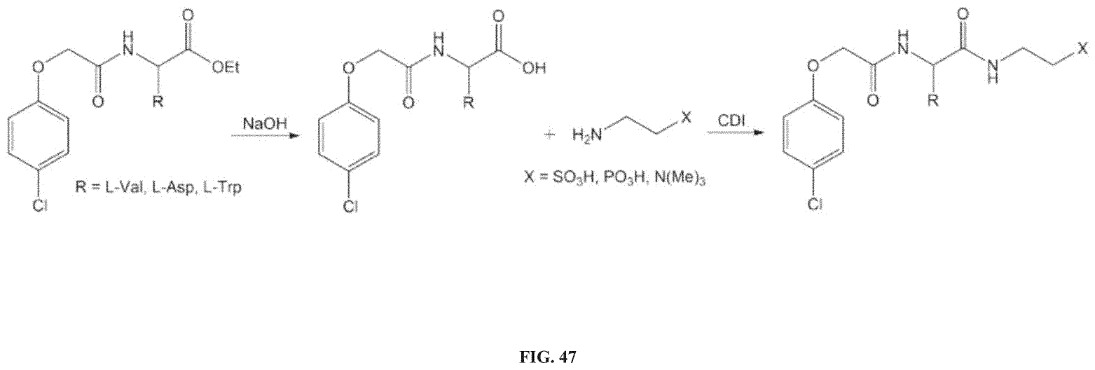

A different approach to making a conjugate more water soluble is to introduce a hydrophilic group (other than carboxylic acid), e.g., attached to a carboxylic acid of an amino acid (e.g., rather than methyl) via an ester or amide bond. Such a hydrophilic group may be, for example, 2-hydroxyethyl, 2-sulfoethyl, 2-phosphoethyl, 2-(trimethylamino)ethyl, or another group comprising one or more hydroxy, amino (e.g., quaternary ammonium), sulfonate, sulfonic acid, phosphonate or phosphonic acid groups. An exemplary synthesis of some such conjugates is depicted schematically in FIG. 47.

The effect of the conjugates on root formation is then optionally assessed according to procedures such as described in any of Examples 2-12.

### Example 14

### Additional Conjugates

Additional conjugates are prepared according to procedures such as described in Example 1, 11, 12 and/or 13 hereinabove, except that 2,4-dichlorophenoxyacetic acid, 2,4,5-trichlorophenoxyacetic acid, 2-(2,4,5-trichlorophenoxy)propionic acid, 4-(4-chloro-2-methylphenoxy)butanoic acid, 4-(4-chlorophenoxy)butanoic acid, 4-(2,4-dichlorophenoxy)butanoic acid, 4-(2,4,5-trichlorophenoxy)butanoic acid, 3,5,6-trichloro-2-pyridinyloxyacetic acid, 3,6-dichloro-2-methoxybenzoic acid (dicamba), 4-amino-3,5,6-trichloro2-pyridinecarboxylic acid (picloram) or indoleacetic acid is conjugated instead of 4-CPA, MCPA, 2-DP and NAA. Each of the 4 auxin analogs is conjugated to various amines by an amide bond or to an alcohol (methanol) by an ester bond.

In some embodiments, the amino acid derivative L-Val-methyl ester is conjugated to 2,4-dichlorophenoxyacetic acid, 2,4,5-trichlorophenoxyacetic acid, 2-(2,4,5-trichlorophenoxy)propionic acid, 4-(4-chloro-2-methylphenoxy)butanoic acid, 4-(4-chlorophenoxy)butanoic acid, 4-(2,4-dichlorophenoxy)butanoic acid, 4-(2,4,5-trichlorophenoxy)butanoic acid, 3,5,6-trichloro-2-pyridinyloxyacetic acid, dicamba, picloram, and/or indoleacetic acid. The methyl ester of the conjugate(s) may optionally be hydrolyzed to form an L-Val sodium salt, according to procedures described herein.

The effect of the conjugates on root formation is then optionally assessed according to procedures such as described in any of Examples 2-12.

### Example 15

### Effect of Conjugates on Fruit Size and Flowering

Conjugates are prepared according to procedures such as described in Example 1, 11, 12, 13 and/or 14 hereinabove.

The effect of the conjugates on flowering is optionally assessed by contacting (e.g., by spraying) plants with a conjugate before and/or during flowering, and determining the effect of the conjugate on the number of flowers.

The effect of the conjugates on fruit size is optionally assessed by contacting fruiting plants with a conjugate before and/or during flowering, as described hereinabove, and/or by contacting (e.g., by spraying) plants with a conjugate during fruit development. The effect of the conjugate on the size of fruit which develops after treatment is then determined.

### Example 16

### Effect of Conjugates on Grafting Unification

Conjugates are prepared according to procedures such as described in Example 1, 11, 12, 13 and/or 14 hereinabove.

The effect of the conjugates on grafting unification is optionally assessed by contacting scions with a conjugate before, during and/or after grafting onto rootstocks (for example, avocado scions and rootstocks), and determining the effect of the conjugate on the percentage of successful grafting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of enhancing formation and/or growth of an adventitious root in a plant and/or plant tissue, the method comprising contacting at least a portion of the plant and/or plant tissue with a compound having Formula I: 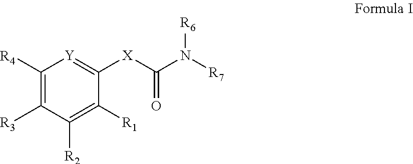
wherein:
X is selected from the group consisting of a bond, CH₂, —O—CH₂— and —O—CH₂CH₂CH₂—;
Y is CR₅ or N;
R₁-R₅ are each individually selected from the group consisting of hydrogen, chloro, methyl, methoxy and amino, or alternatively, R₄ and R₅ together form a six-membered aromatic ring;
R₆ is selected from the group consisting of aryl, heteroaryl, alkyl, alkenyl and alkynyl; and
R₇ is selected from the group consisting of hydrogen and alkyl,
or alternatively, R₆ and R₇ together form a five- or six-membered heteroalicyclic ring,
thereby enhancing formation and/or growth of an adventitious root.

2. The method of claim 1, wherein Y is N.

3. The method of claim 1, wherein X is selected from the group consisting of —O—CH₂— and —O—CH₂CH₂CH₂—.

4. The method of claim 1, wherein X is a bond.

5. The method of claim 1, wherein Y is CR₅, R₄ and R₅ together form said six-membered aromatic ring, and X is CH₂.

6. The method of claim 1, wherein R₆ has Formula II: 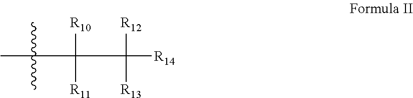
wherein:
R₁₀ and R₁₁ are each selected from the group consisting of hydrogen, alkyl, alkenyl, alkynyl, cycloalkyl, aryl, heteroaryl, heteroalicyclic, carbonyl, thiocarbonyl, C-amido, and C-carboxy; and
R₁₂-R₁₄ are each individually selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

7. The method of claim 6, wherein R₁₀ is selected from the group consisting of —C(═O)OCH₃, —C(═O)OH or a salt thereof, and —C(═O)NH—(CH₂)₂-R₁₈, wherein R₁₈ is an ionic group.

8. A composition for enhancing formation and/or growth of an adventitious root in a plant and/or plant tissue, or for promoting grafting unification, enhancing fruit size and/or reducing flowering in a plant, the composition comprising:
a) a compound having Formula I: 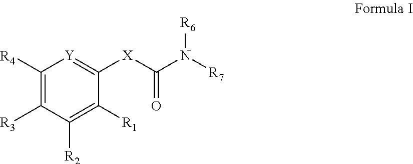
wherein:
X is selected from the group consisting of a bond, CH₂, —O—CH₂— and —O—CH₂CH₂CH₂—;
Y is CR₅ or N;
R₁-R₅ are each individually selected from the group consisting of hydrogen, chloro, methyl, methoxy and amino;
R₆ is selected from the group consisting of aryl, heteroaryl, alkyl, alkenyl and alkynyl; and
R₇ is selected from the group consisting of hydrogen and alkyl,
or alternatively, R₆ and R₇ together form a five- or six-membered heteroalicyclic ring; and
b) a horticulturally acceptable carrier.

9. The composition of claim 8, wherein Y is N.

10. The composition of claim 8, wherein X is selected from the group consisting of —O—CH₂— and —O—CH₂CH₂CH₂—.

11. The composition of claim 8, wherein X is a bond.

12. The composition of claim 8, wherein Y is CR₅, R₄ and R₅ together form said six-membered aromatic ring, and X is CH₂.

13. The composition of claim 8, wherein R₆ has Formula II: 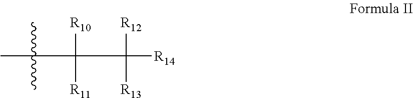
wherein:
R₁₀ and R₁₁ are each selected from the group consisting of hydrogen, alkyl, alkenyl, alkynyl, cycloalkyl, aryl, heteroaryl, heteroalicyclic, carbonyl, thiocarbonyl, C-amido, and C-carboxy; and
R₁₂-R₁₄ are each individually selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

14. A method of promoting grafting unification, enhancing fruit size and/or of reducing flowering in a plant, the method comprising contacting at least a portion of the plant with a compound having Formula I: 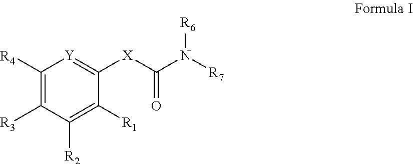
wherein:
X is selected from the group consisting of a bond, CH₂, —O—CH₂— and —O—CH₂CH₂CH₂—;
Y is CR₅ or N;
R₁-R₅ are each individually selected from the group consisting of hydrogen, chloro, methyl, methoxy and amino, or alternatively, R₄ and R₅ together form a six-membered aromatic ring;
R₆ is selected from the group consisting of aryl, heteroaryl, alkyl, alkenyl and alkynyl; and
R₇ is selected from the group consisting of hydrogen and alkyl,
or alternatively, R₆ and R₇ together form a five- or six-membered heteroalicyclic ring,
thereby promoting grafting unification, enhancing fruit size and/or reducing flowering.

15. A compound having Formula Ia: 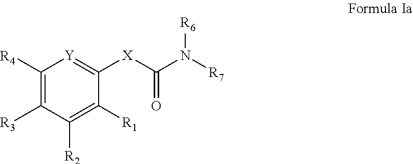
wherein:
X is selected from the group consisting of a bond, CH₂, —O—CH₂— and —O—CH₂CH₂CH₂—;
Y is CR₅ or N;
R₁-R₅ are each individually selected from the group consisting of hydrogen, chloro, methyl, methoxy and amino, or alternatively, R₄ and R₅ together form a six-membered aromatic ring;
R₆ is selected from the group consisting of aryl, alkyl, alkenyl and alkynyl, said alkyl being devoid of a —C(═O)OH substituent at the α-position thereof; and
R₇ is selected from the group consisting of hydrogen and alkyl, wherein when R₇ is alkyl, R₆ is not aryl,
or alternatively, R₆ and R₇ together form a six-membered heteroalicyclic ring.

16. The compound of claim 15, wherein Y is N.

17. The compound of claim 15, wherein X is selected from the group consisting of —O—CH₂— and —O—CH₂CH₂CH₂—.

18. The compound of claim 15, wherein X is a bond.

19. The compound of claim 15, wherein Y is CR₅, R₄ and R₅ together form said six-membered aromatic ring, and X is CH₂.

20. The compound of claim 15, wherein R₆ has Formula II: 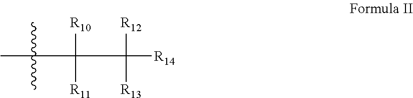
wherein:
R₁₀ and R₁₁ are each selected from the group consisting of hydrogen, alkyl, alkenyl, alkynyl, cycloalkyl, aryl, heteroaryl, heteroalicyclic, carbonyl, thiocarbonyl, C-amido, and C-carboxy, provided that neither R₁₀ nor R₁₁ is-C(═O)OH; and
R₁₂-R₁₄ are each individually selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

21. The compound of claim 20, wherein R₁₀ is selected from the group consisting of —C(═O)OCH₃ and —C(═O)NH—(CH₂)₂-R₁₈, wherein R₁₈ is an ionic group.

22. A method of enhancing formation and/or growth of an adventitious root in a plant and/or plant tissue, the method comprising contacting at least a portion of the plant and/or plant tissue with the compound of claim 15, thereby enhancing formation and/or growth of an adventitious root in a plant and/or plant tissue.

23. The method of claim 1, comprising contacting a base of a plant cutting and at least one leaf of said cutting with said compound.

24. The method of claim 1, further comprising contacting at least a portion of the plant and/or plant tissue with an auxin.

25. A method of promoting grafting unification, enhancing fruit size and/or of reducing flowering in a plant, the method comprising contacting at least a portion of the plant with the compound of claim 15, thereby promoting grafting unification, enhancing fruit size and/or of reducing flowering in a plant.

26. A composition for enhancing formation and/or growth of an adventitious root in a plant and/or plant tissue, the composition comprising:
a) the compound of claim 15; and
b) a horticulturally acceptable carrier.

27. A composition for promoting grafting unification, enhancing fruit size and/or for reducing flowering in a plant, the composition comprising:
a) the compound of claim 15; and
b) a horticulturally acceptable carrier.

28. The composition of claim 8, further comprising an auxin.

29. The composition of claim 28, wherein said auxin comprises indolebutyric acid (IBA).

30. The method of claim 24, wherein said auxin comprises indolebutyric acid (IBA).
